(19) United States
(12) Patent Application Publication
(10) Pub. No.: US 2022/0401391 A1
(43) Pub. Date: Dec. 22, 2022

(54) METHODS AND COMPOSITIONS FOR TREATING PAIN

(71) Applicant: Apkarian Technologies LLC, Chicago, IL (US)

(72) Inventor: A. Vania APKARIAN, Chicago, IL (US)

(21) Appl. No.: 17/610,025

(22) PCT Filed: May 8, 2020
(86) PCT No.: PCT/US2020/032142

§ 371 Date: Nov. 9, 2021

Related U.S. Application Data
(60) Provisional application No. 62/845,782, filed on May 9, 2019.

(51) Int. Cl. A61K 31/167; A61K 31/425; A61K 31/198; A61K 31/192; A61P 29/00

(52) U.S. Cl. CPC A61K 31/167; A61K 31/425; A61K 31/198; A61K 31/192; A61P 29/00

(57) ABSTRACT

The present disclosure features combinations of dopaminergic agents and analgesic agents useful for treating pain. In particular, the combinations feature a low ratio of dopaminergic agent to analgesic agent. The dopaminergic agent can be an agonist of the dopamine receptor D1-like family or the dopamine receptor D2-like family. Such combinations potentiate analgesia to 1) alleviate acute pain, 2) prevent the transition from acute pain to chronic pain, and 3) manage chronic pain.

### STATEMENT REGARDING FEDERALLY SPONSORED RESEARCH

This invention was made with government support under grant number 5R01 DE022746-02 awarded by the National Institute of Dental and Craniofacial Research. The government has certain rights in the invention.

### BACKGROUND OF THE INVENTION

Pain is a complex process associated with actual or potential tissue damage, creating an unpleasant sensory and emotional experience. Pain motivates an individual to withdraw from damaging situations, to protect a damaged body part while it heals, and to avoid similar experiences in the future. Most pain is transient and resolves promptly once the stimulus is removed and the body has healed, but some pain can persist despite removal of the stimulus and apparent healing of the body.

Pain conditions can generally be divided into acute pain and chronic pain. Acute pain usually follows non-neural tissue injury (e.g., tissue damage from surgery or inflammation, or migraine) and is usually transient. In contrast, chronic pain persists long after the physiological environment has recovered from damage associated with acute pain. In some cases, chronic pain develops in the absence of any detectable stimulus, damage, or disease. Chronic pain includes neuropathic pain (e.g., post-surgical and post-herpetic neuralgia), chronic inflammatory pain (e.g., arthritis), and pain of unknown origin (e.g., fibromyalgia). The financial burden associated with chronic pain in the United States is estimated to be greater than $500 billion a year, due to decreased productivity and medical expenses. There is a clear need for effective treatments for acute and chronic pain.

There are many drugs that are known to be useful in the treatment or management of pain. Analgesic agents are those that have a direct effect of alleviating pain. One class of analgesics, nonsteroidal anti-inflammatories (NSAIDs), can be used to relieve acute pain and various chronic pain conditions. NSAIDs have limited efficacy in most cases of chronic pain. Additionally, the analgesic effects of NSAIDs are not well characterized with respect to the relationship between acute and chronic pain, and there is a lack of effective treatments for preventing the transition from acute to chronic pain. Therefore, there remains a need to develop improved therapies for the treatment of acute and chronic pain.

### SUMMARY OF THE INVENTION

The present disclosure provides compositions, methods, and kits for treating acute pain and chronic pain in a subject (e.g., a mammalian subject, such as a human, for example, a human female). The disclosure also features compositions, methods, and kits for preventing the transition from acute pain to chronic pain in a subject.

In a first aspect, the disclosure features a method of reducing pain in a subject by administering an analgesic agent, a first dopaminergic agent, and/or a second dopaminergic agent to the subject, wherein:
(i) the analgesic agent is administered to the subject in an amount of from about 50 mg to about 500 mg per dose;
(ii) the first dopaminergic agent is administered to the subject in an amount of from about 5 mg to about 75 mg per dose; and/or
(iii) the second dopaminergic agent is administered to the subject in an amount of from about 25 mg to about 300 mg per dose.

In another aspect, the disclosure features a method of reducing pain in a subject by administering an analgesic agent, a first dopaminergic agent, and/or a second dopaminergic agent to the subject, wherein:
(i) the analgesic agent is administered to the subject in an amount of from about 150 mg to about 1,500 mg per day;
(ii) the first dopaminergic agent is administered to the subject in an amount of from about 20 mg to about 200 mg per day; and/or
(iii) the second dopaminergic agent is administered to the subject in an amount of from about 100 mg to about 1,000 mg per day.

In a further aspect, the disclosure features a method of reducing pain in a subject by administering an analgesic agent, a first dopaminergic agent, and/or a second dopaminergic agent to the subject, wherein:
(i) the analgesic agent is administered to the subject in an amount of from about 1,000 mg to about 10,000 mg per week;
(ii) the first dopaminergic agent is administered to the subject in an amount of from about 100 mg to about 1,500 mg per week; and/or
(iii) the second dopaminergic agent is administered to the subject in an amount of from about 500 mg to about 5,000 mg per week.

In some embodiments of any of the above aspects of the disclosure, the subject is treated with a single dopaminergic agent (for example, either the “first dopaminergic agent” or the “second dopaminergic agent” as recited above and herein). In such instances, the subject may additionally be administered the remaining dopaminergic agent. For the avoidance of doubt, statements described herein as referring to treatment of a subject with a combination of a first dopaminergic agent and a second dopaminergic agent can also be applied to treatment of a subject using either the first dopaminergic agent or the second dopaminergic agent in isolation (for example, in accordance with a dosing schedule described herein for a first dopaminergic agent or a second dopaminergic agent).

In some embodiments, the analgesic agent is administered to the subject in an amount of from about 50 mg to about 500 mg per dose. For example, the analgesic agent may be administered to the subject in an amount of from about 100 mg to about 400 mg per dose. In some embodiments, the analgesic agent is administered to the subject in an amount of from about 200 mg to about 300 mg per dose. For example, the analgesic agent may be administered to the subject in an amount of from about 225 mg to about 275 mg per dose. In some embodiments, the analgesic agent is administered to the subject in an amount of about 250 mg per dose.

In some embodiments, the analgesic agent is administered to the subject in one or more doses per 12 hours, 24 hours, 36 hours, 48 hours, or week. The analgesic agent may, for example, be administered to the subject in one or more doses per 24 hours. In some embodiments, the analgesic agent is administered to the subject in from one to ten doses per day, such as from one to six doses per day (e.g., 1, 2, 3, 4, 5, or 6 doses per day). In some embodiments, the analgesic agent is administered to the subject in three doses per day.

In some embodiments, the analgesic agent is administered to the subject in an amount of from about 150 mg to about 1,500 mg per day. For example, the analgesic agent may be administered to the subject in an amount of from about 200 mg to about 1,200 mg per day. In some embodiments, the analgesic agent is administered to the subject in an amount of from about 500 mg to about 1.00 mg per day. In some embodiments, the analgesic agent is administered to the subject in an amount of from about 700 mg to about 800 mg per day. In some embodiments, the analgesic agent is administered to the subject in an amount of about 750 mg per day.

In some embodiments, the analgesic agent is administered to the subject in an amount of from about 1,000 mg to about 10,000 mg per week. For example, the analgesic agent may be administered to the subject in an amount of from about 2,000 mg to about 9,000 mg per week. In some embodiments, the analgesic agent is administered to the subject in an amount of from about 3,000 mg to about 7,000 mg per week. In some embodiments, the analgesic agent is administered to the subject in an amount of from about 5,000 mg to about 6,000 mg per week. In some embodiments, the analgesic agent is administered to the subject in an amount of about 5,250 mg per week.

In some embodiments, the analgesic agent is periodically administered to the subject over the course of a treatment period having a duration of from about 1 day to about 1 year. For example, the treatment period may have a duration of from about 1 week to about 24 weeks (e.g., a duration of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks).

In some embodiments, the first dopaminergic agent is administered to the subject in an amount of from about 5 mg to about 75 mg per dose. For example, the first dopaminergic agent may be administered to the subject in an amount of from about 7.5 mg to about 25 mg per dose (e.g., in an amount of from about 9 mg to about 20 mg per dose, such as in an amount of from about 10 mg to about 15 mg per dose, such as in an amount of about 12.5 mg per dose). In some embodiments, the first dopaminergic agent is administered to the subject in an amount of from about 15 mg to about 50 mg per dose (e.g., in an amount of from about 17 mg to about 35 mg per dose, such as in an amount of from about 20 mg to about 30 mg per dose, such as in an amount of about 25 mg per dose). In some embodiments, the first dopaminergic agent is administered to the subject in an amount of from about 25 mg to about 75 mg per dose (e.g., in an amount of from about 45 mg to about 65 mg per dose, such as in an amount of from about 40 mg to about 60 mg per dose, such as in an amount of about 50 mg per dose).

In some embodiments, the first dopaminergic agent is administered to the subject in an amount of from about 7.5 mg to about 25 mg per dose (e.g., in an amount of from about 9 mg to about 20 mg per dose, such as in an amount of from about 10 mg to about 15 mg per dose, such as in an amount of about 12.5 mg per dose) and is subsequently administered to the subject in a higher amount (e.g., in an amount of from about 17 mg to about 35 mg per dose, such as in an amount of from about 20 mg to about 30 mg per dose, such as in an amount of about 25 mg per dose; or in an amount of from about 45 mg to about 65 mg per dose, such as in an amount of from about 40 mg to about 60 mg per dose, such as in an amount of about 50 mg per dose), for example, if the subject does not respond to the lower dosing regimen after a treatment period (e.g., after a treatment period of from about 1 week to about 24 weeks, such as after a treatment period of about 4 weeks).

In some embodiments, the first dopaminergic agent is administered to the subject in an amount of from about 15 mg to about 50 mg per dose (e.g., in an amount of from about 17 mg to about 35 mg per dose, such as in an amount of from about 20 mg to about 30 mg per dose, such as in an amount of about 25 mg per dose) and is subsequently administered to the subject in a higher amount (e.g., in an amount of from about 45 mg to about 65 mg per dose, such as in an amount of from about 40 mg to about 60 mg per dose, such as in an amount of about 50 mg per dose), for example, if the subject does not respond to the lower dosing regimen after a treatment period (e.g., after a treatment period of from about 1 week to about 24 weeks, such as after a treatment period of about 4 weeks). In some embodiments, the first dopaminergic agent is administered to the subject in an amount of from about 15 mg to about 50 mg per dose (e.g., in an amount of from about 17 mg to about 35 mg per dose, such as in an amount of from about 20 mg to about 30 mg per dose, such as in an amount of about 25 mg per dose) and is subsequently administered to the subject in a lower amount (e.g., in an amount of from about 9 mg to about 20 mg per dose, such as in an amount of from about 10 mg to about 15 mg per dose, such as in an amount of about 12.5 mg per dose), for example, if the subject does respond to the higher dosing regimen after a treatment period (e.g., after a treatment period of from about 1 week to about 24 weeks, such as after a treatment period of about 4 weeks).

In some embodiments, the first dopaminergic agent is administered to the subject in an amount of from about 25 mg to about 75 mg per dose (e.g., in an amount of from about 45 mg to about 65 mg per dose, such as in an amount of from about 40 mg to about 60 mg per dose, such as in an amount of about 50 mg per dose) and is subsequently administered to the subject in a lower amount (e.g., in an amount of from about 9 mg to about 20 mg per dose, such as in an amount of from about 10 mg to about 15 mg per dose, such as in an amount of about 12.5 mg per dose; or in an amount of from about 17 mg to about 35 mg per dose, such as in an amount of from about 20 mg to about 30 mg per dose, such as in an amount of about 25 mg per dose), for example, if the subject does respond to the higher dosing regimen after a treatment period (e.g., after a treatment period of from about 1 week to about 24 weeks, such as after a treatment period of about 4 weeks).

In some embodiments, the first dopaminergic agent is administered to the subject in one or more doses per 12 hours, 24 hours, 36 hours, 48 hours, or week. The first dopaminergic agent may, for example, be administered to the subject in one or more doses per 24 hours. In some embodiments, the first dopaminergic agent is administered to the subject in from one to ten doses per day, such as from one to six doses per day (e.g., 1, 2, 3, 4, 5, or 6 doses per day). In some embodiments, the first dopaminergic agent is administered to the subject in three doses per day.

In some embodiments, the first dopaminergic agent is administered to the subject in an amount of from about 20 mg to about 200 mg per day. For example, the first dopaminergic agent may be administered to the subject in an amount of from about 25 mg to about 175 mg per day (e.g., in an amount of from about 30 mg to about 50 mg per day, such as in an amount of from about 35 mg to about 40 mg per day, such as in an amount of about 37.5 mg per day). In some embodiments, the first dopaminergic agent is administered to the subject in an amount of from about 40 mg to about 100 mg per day (e.g., in an amount of from about 50 mg to about 90 mg per day, such as in an amount of from about 60 mg to about 80 mg per day, such as in an amount of about 75 mg per day). In some embodiments, the first dopaminergic agent is administered to the subject in an amount of from about 75 mg to about 200 mg per day (e.g., in an amount of from about 125 mg to about 175 mg per day, such as in an amount of from about 140 mg to about 160 mg per day, such as in an amount of about 150 mg per day).

In some embodiments, the first dopaminergic agent is administered to the subject in an amount of from about 25 mg to about 175 mg per day (e.g., in an amount of from about 30 mg to about 50 mg per day, such as in an amount of from about 35 mg to about 40 mg per day, such as in an amount of about 37.5 mg per day), and is subsequently administered to the subject in a higher amount (e.g., in an amount of from about 50 mg to about 90 mg per day, such as in an amount of from about 60 mg to about 80 mg per day, such as in an amount of about 75 mg per day; or in an amount of from about 125 mg to about 175 mg per day, such as in an amount of from about 140 mg to about 160 mg per day, such as in an amount of about 150 mg per day), for example, if the subject does not respond to the lower dosing regimen after a treatment period (e.g., after a treatment period of from about 1 week to about 24 weeks, such as after a treatment period of about 4 weeks).

In some embodiments, the first dopaminergic agent is administered to the subject in an amount of from about 40 mg to about 100 mg per day (e.g., in an amount of from about 50 mg to about 90 mg per day, such as in an amount of from about 60 mg to about 80 mg per day, such as in an amount of about 75 mg per day), and is subsequently administered to the subject in a higher amount (e.g., in an amount of from about 125 mg to about 175 mg per day, such as in an amount of from about 140 mg to about 160 mg per day, such as in an amount of about 150 mg per day), for example, if the subject does not respond to the lower dosing regimen after a treatment period (e.g., after a treatment period of from about 1 week to about 24 weeks, such as after a treatment period of about 4 weeks). In some embodiments, the first dopaminergic agent is administered to the subject in an amount of from about 40 mg to about 100 mg per day (e.g., in an amount of from about 50 mg to about 90 mg per day, such as in an amount of from about 60 mg to about 80 mg per day, such as in an amount of about 75 mg per day), and is subsequently administered to the subject in a lower amount (e.g., in an amount of from about 30 mg to about 50 mg per day, such as in an amount of from about 35 mg to about 40 mg per day, such as in an amount of about 37.5 mg per day), for example, if the subject does respond to the higher dosing regimen after a treatment period (e.g., after a treatment period of from about 1 week to about 24 weeks, such as after a treatment period of about 4 weeks).

In some embodiments, the first dopaminergic agent is administered to the subject in an amount of from about 75 mg to about 200 mg per day (e.g., in an amount of from about 125 mg to about 175 mg per day, such as in an amount of from about 140 mg to about 160 mg per day, such as in an amount of about 150 mg per day), and is subsequently administered to the subject in a lower amount (e.g., in an amount of from about 30 mg to about 50 mg per day, such as in an amount of from about 35 mg to about 40 mg per day, such as in an amount of about 37.5 mg per day; or in an amount of from about 50 mg to about 90 mg per day, such as in an amount of from about 60 mg to about 80 mg per day, such as in an amount of about 75 mg per day), for example, if the subject does respond to the higher dosing regimen after a treatment period (e.g., after a treatment period of from about 1 week to about 24 weeks, such as after a treatment period of about 4 weeks).

In some embodiments, the first dopaminergic agent is administered to the subject in an amount of from about 100 mg to about 1,500 mg per week. For example, the first dopaminergic agent may be administered to the subject in an amount of from about 200 mg to about 500 mg per week (e.g., in an amount of from about 225 mg to about 300 mg per week, such as in an amount of from about 230 mg to about 280 mg per week, such as in an amount of about 262.5 mg per week). In some embodiments, the first dopaminergic agent is administered to the subject in an amount of from about 300 mg to about 700 mg per week (e.g., in an amount of from about 400 mg to about 600 mg per week, such as in an amount of from about 500 mg to about 550 mg per week, such as in an amount of about 525 mg per week). In some embodiments, the first dopaminergic agent is administered to the subject in an amount of from about 500 mg to about 1,500 mg per week (e.g., in an amount of from about 750 mg to about 1,300 mg per week, such as in an amount of from about 900 mg to about 1,200 mg per week, such as in an amount of about 1,050 mg per week).

In some embodiments, the first dopaminergic agent is administered to the subject in an amount of from about 200 mg to about 500 mg per week (e.g., in an amount of from about 225 mg to about 300 mg per week, such as in an amount of from about 230 mg to about 280 mg per week, such as in an amount of about 262.5 mg per week) and is subsequently administered to the subject in a higher amount (e.g., in an amount of from about 400 mg to about 600 mg per week, such as in an amount of from about 500 mg to about 550 mg per week, such as in an amount of about 525 mg per week; or in an amount of from about 750 mg to about 1,300 mg per week, such as in an amount of from about 900 mg to about 1,200 mg per week, such as in an amount of about 1,050 mg per week), for example, if the subject does not respond to the lower dosing regimen after a treatment period (e.g., after a treatment period of from about 1 week to about 24 weeks, such as after a treatment period of about 4 weeks).

In some embodiments, the first dopaminergic agent is administered to the subject in an amount of from about 300 mg to about 700 mg per week (e.g., in an amount of from about 400 mg to about 600 mg per week, such as in an amount of from about 500 mg to about 550 mg per week, such as in an amount of about 525 mg per week) and is subsequently administered to the subject in a higher amount (e.g., in an amount of from about 750 mg to about 1,300 mg per week, such as in an amount of from about 900 mg to about 1,200 mg per week, such as in an amount of about 1,050 mg per week), for example, if the subject does not respond to the lower dosing regimen after a treatment period (e.g., after a treatment period of from about 1 week to about 24 weeks, such as after a treatment period of about 4 weeks). In some embodiments, the first dopaminergic agent is administered to the subject in an amount of from about 300 mg to about 700 mg per week (e.g., in an amount of from about 400 mg to about 600 mg per week, such as in an amount of from about 500 mg to about 550 mg per week, such as in an amount of about 525 mg per week) and is subsequently administered to the subject in a lower amount (e.g., in an amount of from about 225 mg to about 300 mg per week, such as in an amount of from about 230 mg to about 280 mg per week, such as in an amount of about 262.5 mg per week), for example, if the subject does respond to the higher dosing regimen after a treatment period (e.g., after a treatment period of from about 1 week to about 24 weeks, such as after a treatment period of about 4 weeks).

In some embodiments, the first dopaminergic agent is administered to the subject in an amount of from about 500 mg to about 1,500 mg per week (e.g., in an amount of from about 750 mg to about 1,300 mg per week, such as in an amount of from about 900 mg to about 1,200 mg per week, such as in an amount of about 1,050 mg per week) and is subsequently administered to the subject in a lower amount (e.g., in an amount of from about 225 mg to about 300 mg per week, such as in an amount of from about 230 mg to about 280 mg per week, such as in an amount of about 262.5 mg per week; or in an amount of from about 400 mg to about 600 mg per week, such as in an amount of from about 500 mg to about 550 mg per week, such as in an amount of about 525 mg per week), for example, if the subject does respond to the higher dosing regimen after a treatment period (e.g., after a treatment period of from about 1 week to about 24 weeks, such as after a treatment period of about 4 weeks).

In some embodiments, the first dopaminergic agent is periodically administered to the subject over the course of a treatment period having a duration of from about 1 day to about 1 year. For example, the treatment period may have a duration of from about 1 week to about 24 weeks (e.g., a duration of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks).

In some embodiments, the second dopaminergic agent is administered to the subject in an amount of from about 25 mg to about 300 mg per dose. For example, the second dopaminergic agent may be administered to the subject in an amount of from about 35 mg to about 100 mg per dose (e.g., in an amount of from about 40 mg to about 75 mg per dose, such as in an amount of from about 45 mg to about 55 mg per dose, such as in an amount of about 50 mg per dose). In some embodiments, the second dopaminergic agent is administered to the subject in an amount of from about 50 mg to about 150 mg per dose (e.g., in an amount of from about 75 mg to about 125 mg per dose, such as in an amount of from about 85 mg to about 110 mg per dose, such as in an amount of about 100 mg per dose). In some embodiments, the second dopaminergic agent is administered to the subject in an amount of from about 150 mg to about 250 mg per dose (e.g., in an amount of from about 175 mg to about 225 mg per dose, such as in an amount of from about 190 mg to about 210 mg per dose, such as in an amount of about 200 mg per dose).

In some embodiments, the second dopaminergic agent is administered to the subject in an amount of from about 35 mg to about 100 mg per dose (e.g., in an amount of from about 40 mg to about 75 mg per dose, such as in an amount of from about 45 mg to about 55 mg per dose, such as in an amount of about 50 mg per dose), and is subsequently administered to the subject in a higher amount (e.g., in an amount of from about 75 mg to about 125 mg per dose, such as in an amount of from about 85 mg to about 110 mg per dose, such as in an amount of about 100 mg per dose; or in an amount of from about 175 mg to about 225 mg per dose, such as in an amount of from about 190 mg to about 210 mg per dose, such as in an amount of about 200 mg per dose), for example, if the subject does not respond to the lower dosing regimen after a treatment period (e.g., after a treatment period of from about 1 week to about 24 weeks, such as after a treatment period of about 4 weeks).

In some embodiments, the second dopaminergic agent is administered to the subject in an amount of from about 50 mg to about 150 mg per dose (e.g., in an amount of from about 75 mg to about 125 mg per dose, such as in an amount of from about 85 mg to about 110 mg per dose, such as in an amount of about 100 mg per dose), and is subsequently administered to the subject in a higher amount (e.g., in an amount of from about 175 mg to about 225 mg per dose, such as in an amount of from about 190 mg to about 210 mg per dose, such as in an amount of about 200 mg per dose), for example, if the subject does not respond to the lower dosing regimen after a treatment period (e.g., after a treatment period of from about 1 week to about 24 weeks, such as after a treatment period of about 4 weeks). In some embodiments, the second dopaminergic agent is administered to the subject in an amount of from about 50 mg to about 150 mg per dose (e.g., in an amount of from about 75 mg to about 125 mg per dose, such as in an amount of from about 85 mg to about 110 mg per dose, such as in an amount of about 100 mg per dose), and is subsequently administered to the subject in a lower amount (e.g., in an amount of from about 40 mg to about 75 mg per dose, such as in an amount of from about 45 mg to about 55 mg per dose, such as in an amount of about 50 mg per dose), for example, if the subject does respond to the higher dosing regimen after a treatment period (e.g., after a treatment period of from about 1 week to about 24 weeks, such as after a treatment period of about 4 weeks).

In some embodiments, the second dopaminergic agent is administered to the subject in an amount of from about 150 mg to about 250 mg per dose (e.g., in an amount of from about 175 mg to about 225 mg per dose, such as in an amount of from about 190 mg to about 210 mg per dose, such as in an amount of about 200 mg per dose), and is subsequently administered to the subject in a lower amount (e.g., in an amount of from about 40 mg to about 75 mg per dose, such as in an amount of from about 45 mg to about 55 mg per dose, such as in an amount of about 50 mg per dose; or in an amount of from about 75 mg to about 125 mg per dose, such as in an amount of from about 85 mg to about 110 mg per dose, such as in an amount of about 100 mg per dose), for example, if the subject does respond to the higher dosing regimen after a treatment period (e.g., after a treatment period of from about 1 week to about 24 weeks, such as after a treatment period of about 4 weeks).

In some embodiments, the second dopaminergic agent is administered to the subject in one or more doses per 12 hours, 24 hours, 36 hours, 48 hours, or week. The second dopaminergic agent may, for example, be administered to the subject in one or more doses per 24 hours. In some embodiments, the second dopaminergic agent is administered to the subject in from one to ten doses per day, such as from one to six doses per day (e.g., 1, 2, 3, 4, 5, or 6 doses per day). In some embodiments, the second dopaminergic agent is administered to the subject in three doses per day.

In some embodiments, the second dopaminergic agent is administered to the subject in an amount of from about 100 mg to about 1,000 mg per day. For example, the second dopaminergic agent may be administered to the subject in an amount of from about 100 mg to about 400 mg per day (e.g., in an amount of from about 125 mg to about 200 mg per day, such as in an amount of from about 145 mg to about 155 mg per day, such as in an amount of about 150 mg per day). In some embodiments, the second dopaminergic agent is administered to the subject in an amount of from about 200 mg to about 400 mg per day (e.g., in an amount of from about 250 mg to about 350 mg per day, such as in an amount of from about 275 mg to about 325 mg per day, such as in an amount of about 300 mg per day). In some embodiments, the second dopaminergic agent is administered to the subject in an amount of from about 500 mg to about 700 mg per day (e.g., in an amount of from about 550 mg to about 650 mg per day, such as in an amount of from about 575 mg to about 625 mg per day, such as in an amount of about 600 mg per day).

In some embodiments, the second dopaminergic agent is administered to the subject in an amount of from about 100 mg to about 400 mg per day (e.g., in an amount of from about 125 mg to about 200 mg per day, such as in an amount of from about 145 mg to about 155 mg per day, such as in an amount of about 150 mg per day), and is subsequently administered to the subject in a higher amount (e.g., in an amount of from about 250 mg to about 350 mg per day, such as in an amount of from about 275 mg to about 325 mg per day, such as in an amount of about 300 mg per day; or in an amount of from about 550 mg to about 650 mg per day, such as in an amount of from about 575 mg to about 625 mg per day, such as in an amount of about 600 mg per day), for example, if the subject does not respond to the lower dosing regimen after a treatment period (e.g., after a treatment period of from about 1 week to about 24 weeks, such as after a treatment period of about 4 weeks).

In some embodiments, the second dopaminergic agent is administered to the subject in an amount of from about 200 mg to about 400 mg per day (e.g., in an amount of from about 250 mg to about 350 mg per day, such as in an amount of from about 275 mg to about 325 mg per day, such as in an amount of about 300 mg per day), and is subsequently administered to the subject in a higher amount (e.g., in an amount of from about 550 mg to about 650 mg per day, such as in an amount of from about 575 mg to about 625 mg per day, such as in an amount of about 600 mg per day), for example, if the subject does not respond to the lower dosing regimen after a treatment period (e.g., after a treatment period of from about 1 week to about 24 weeks, such as after a treatment period of about 4 weeks). In some embodiments, the second dopaminergic agent is administered to the subject in an amount of from about 200 mg to about 400 mg per day (e.g., in an amount of from about 250 mg to about 350 mg per day, such as in an amount of from about 275 mg to about 325 mg per day, such as in an amount of about 300 mg per day), and is subsequently administered to the subject in a lower amount (e.g., in an amount of from about 125 mg to about 200 mg per day, such as in an amount of from about 145 mg to about 155 mg per day, such as in an amount of about 150 mg per day), for example, if the subject does respond to the higher dosing regimen after a treatment period (e.g., after a treatment period of from about 1 week to about 24 weeks, such as after a treatment period of about 4 weeks).

In some embodiments, the second dopaminergic agent is administered to the subject in an amount of from about 500 mg to about 700 mg per day (e.g., in an amount of from about 550 mg to about 650 mg per day, such as in an amount of from about 575 mg to about 625 mg per day, such as in an amount of about 600 mg per day), and is subsequently administered to the subject in a lower amount (e.g., in an amount of from about 125 mg to about 200 mg per day, such as in an amount of from about 145 mg to about 155 mg per day, such as in an amount of about 150 mg per day; or in an amount of from about 250 mg to about 350 mg per day, such as in an amount of from about 275 mg to about 325 mg per day, such as in an amount of about 300 mg per day), for example, if the subject does respond to the higher dosing regimen after a treatment period (e.g., after a treatment period of from about 1 week to about 24 weeks, such as after a treatment period of about 4 weeks).

In some embodiments, the second dopaminergic agent is administered to the subject in an amount of from about 500 mg to about 5,000 mg per week. For example, the second dopaminergic agent may be administered to the subject in an amount of from about 750 mg to about 1,300 mg per week (e.g., in an amount of from about 800 mg to about 1,200 mg per week, such as in an amount of from about 900 mg to about 1,100 mg per week, such as in an amount of about 1,050 mg per week). In some embodiments, the second dopaminergic agent is administered to the subject in an amount of from about 1,500 mg to about 2,500 mg per week (e.g., in an amount of from about 1,700 mg to about 2,300 mg per week, such as in an amount of from about 1,900 mg to about 2,200 mg per week, such as in an amount of about 2,100 mg per week). In some embodiments, the second dopaminergic agent is administered to the subject in an amount of from about 3,500 mg to about 4,500 mg per week (e.g., in an amount of from about 3,700 mg to about 4,400 mg per week, such as in an amount of from about 4,100 mg to about 4,300 mg per week, such as in an amount of about 4,200 mg per week).

In some embodiments, the second dopaminergic agent is administered to the subject in an amount of from about 750 mg to about 1,300 mg per week (e.g., in an amount of from about 800 mg to about 1,200 mg per week, such as in an amount of from about 900 mg to about 1,100 mg per week, such as in an amount of about 1,050 mg per week), and is subsequently administered to the subject in a higher amount (e.g., in an amount of from about 1,700 mg to about 2,300 mg per week, such as in an amount of from about 1,900 mg to about 2,200 mg per week, such as in an amount of about 2,100 mg per week; or in an amount of from about 3,700 mg to about 4,400 mg per week, such as in an amount of from about 4,100 mg to about 4,300 mg per week, such as in an amount of about 4,200 mg per week), for example, if the subject does not respond to the lower dosing regimen after a treatment period (e.g., after a treatment period of from about 1 week to about 24 weeks, such as after a treatment period of about 4 weeks).

In some embodiments, the second dopaminergic agent is administered to the subject in an amount of from about 1,500 mg to about 2,500 mg per week (e.g., in an amount of from about 1,700 mg to about 2,300 mg per week, such as in an amount of from about 1,900 mg to about 2,200 mg per week, such as in an amount of about 2,100 mg per week), and is subsequently administered to the subject in a higher amount (e.g., in an amount of from about 3,700 mg to about 4,400 mg per week, such as in an amount of from about 4,100 mg to about 4,300 mg per week, such as in an amount of about 4,200 mg per week), for example, if the subject does not respond to the lower dosing regimen after a treatment period (e.g., after a treatment period of from about 1 week to about 24 weeks, such as after a treatment period of about 4 weeks). In some embodiments, the second dopaminergic agent is administered to the subject in an amount of from about 1,500 mg to about 2,500 mg per week (e.g., in an amount of from about 1,700 mg to about 2,300 mg per week, such as in an amount of from about 1,900 mg to about 2,200 mg per week, such as in an amount of about 2,100 mg per week), and is subsequently administered to the subject in a lower amount (e.g., in an amount of from about 800 mg to about 1,200 mg per week, such as in an amount of from about 900 mg to about 1,100 mg per week, such as in an amount of about 1,050 mg per week), for example, if the subject does respond to the higher dosing regimen after a treatment period (e.g., after a treatment period of from about 1 week to about 24 weeks, such as after a treatment period of about 4 weeks).

In some embodiments, the second dopaminergic agent is administered to the subject in an amount of from about 3,500 mg to about 4,500 mg per week (e.g., in an amount of from about 3,700 mg to about 4,400 mg per week, such as in an amount of from about 4,100 mg to about 4,300 mg per week, such as in an amount of about 4,200 mg per week), and is subsequently administered to the subject in a lower amount (e.g., in an amount of from about 800 mg to about 1,200 mg per week, such as in an amount of from about 900 mg to about 1,100 mg per week, such as in an amount of about 1,050 mg per week; or in an amount of from about 1,700 mg to about 2,300 mg per week, such as in an amount of from about 1,900 mg to about 2,200 mg per week, such as in an amount of about 2,100 mg per week), for example, if the subject does respond to the higher dosing regimen after a treatment period (e.g., after a treatment period of from about 1 week to about 24 weeks, such as after a treatment period of about 4 weeks).

In some embodiments, the second dopaminergic agent is periodically administered to the subject over the course of a treatment period having a duration of from about 1 day to about 1 year. For example, the treatment period may have a duration of from about 1 week to about 24 weeks (e.g., a duration of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks).

In some embodiments, the first dopaminergic agent is administered to the subject:
(i) in an amount of from about 7.5 mg to about 25 mg per dose during a first treatment period;
(ii) in an amount of from about 15 mg to about 50 mg per dose during a second treatment period; and
(iii) in an amount of from about 25 mg to about 75 mg per dose during a third treatment period,
   optionally wherein the first dopaminergic agent is administered to the subject in three doses per day during each treatment period.

In some embodiments, the first dopaminergic agent is administered to the subject:
(i) in an amount of from about 9 mg to about 20 mg per dose during a first treatment period;
(ii) in an amount of from about 17 mg to about 35 mg per dose during a second treatment period; and
(iii) in an amount of from about 45 mg to about 65 mg per dose during a third treatment period,
   optionally wherein the first dopaminergic agent is administered to the subject in three doses per day during each treatment period.

In some embodiments, the first dopaminergic agent is administered to the subject:
(i) in an amount of from about 10 mg to about 15 mg per dose during a first treatment period;
(ii) in an amount of from about 20 mg to about 30 mg per dose during a second treatment period; and
(iii) in an amount of from about 40 mg to about 60 mg per dose during a third treatment period,
   optionally wherein the first dopaminergic agent is administered to the subject in three doses per day during each treatment period.

In some embodiments, the first dopaminergic agent is administered to the subject:
(i) in an amount of about 12.5 mg per dose during a first treatment period;
(ii) in an amount of about 25 mg per dose during a second treatment period; and
(iii) in an amount of about 50 mg per dose during a third treatment period,
   optionally wherein the first dopaminergic agent is administered to the subject in three doses per day during each treatment period.

In some embodiments, the first, second, and/or third treatment period of administration of the first dopaminergic agent has a duration of from about 1 week to about 24 weeks (e.g., a duration of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks).

In some embodiments, the second dopaminergic agent is administered to the subject:
(i) in an amount of from about 35 mg to about 100 mg per dose during a first treatment period;
(ii) in an amount of from about 50 mg to about 150 mg per dose during a second treatment period; and
(iii) in an amount of from about 150 mg to about 250 mg per dose during a third treatment period,
   optionally wherein the second dopaminergic agent is administered to the subject in three doses per day during each treatment period.

In some embodiments, the second dopaminergic agent is administered to the subject:
(i) in an amount of from about 40 mg to about 75 mg per dose during a first treatment period;
(ii) in an amount of from about 75 mg to about 125 mg per dose during a second treatment period; and
(iii) in an amount of from about 175 mg to about 225 mg per dose during a third treatment period,
   optionally wherein the second dopaminergic agent is administered to the subject in three doses per day during each treatment period.

In some embodiments, the second dopaminergic agent is administered to the subject:
(i) in an amount of from about 45 mg to about 55 mg per dose during a first treatment period;
(ii) in an amount of from about 85 mg to about 110 mg per dose during a second treatment period; and
(iii) in an amount of from about 190 mg to about 210 mg per dose during a third treatment period,
   optionally wherein the second dopaminergic agent is administered to the subject in three doses per day during each treatment period.

In some embodiments, the second dopaminergic agent is administered to the subject:
(i) in an amount of about 50 mg per dose during a first treatment period;
(ii) in an amount of about 100 mg per dose during a second treatment period; and
(iii) in an amount of about 200 mg per dose during a third treatment period,
   optionally wherein the second dopaminergic agent is administered to the subject in three doses per day during each treatment period.

In some embodiments, the first, second, and/or third treatment period of administration of the second dopaminergic agent has a duration of from about 1 week to about 24 weeks (e.g., a duration of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks).

In some embodiments, the analgesic agent, first dopaminergic agent, and second dopaminergic agent are administered to the subject in a single pharmaceutical composition. In some embodiments, the analgesic agent is administered to the subject in a first pharmaceutical composition and the first dopaminergic agent and second dopaminergic agent are administered to the subject in a separate pharmaceutical composition.

In some embodiments, the analgesic agent is a non-steroidal anti-inflammatory drug (NSAID), such as naproxen, aceclofenac, acemetacin, acetaminophen, aloxiprin, aspirin, benorilate, bromfenac, celecoxib, deracoxib, diclofenac, diflunisal, ethenzamide, etodolac, etofenamate, etoricoxib, fenbufen, fenoprofen, flufenamic acid, flurbiprofen, lonazolac, lornoxicam, ibuprofen, indomethacin, isoxicam, kebuzone, ketoprofen, ketorolac, licofelone, loxoprofen, lumiracoxib, meclofenamic acid, mefenamic acid, meloxicam, metamizol, mofebutazone, naproxen, nabumetone, niflumic acid, nimesulide, oxaprozin, oxyphenbutazone, parecoxib, phenidone, phenylbutazone, piroxicam, propacetamol, propyphenazone, rofecoxib, salicylamide, sulfinpyrazone, sulindac, suprofen, tiaprofenic acid, tenoxicam, tolmetin, or valdecoxib. In some embodiments, the analgesic agent is naproxen (e.g., ALEVE®, ACCORD®, ANAPROX®, ANTALGIN®, APRANAX®, FEMINAX ULTRA®, FLANAX®, INZA®, MAXIDOL®, MIDOL EXTENDED RELIEF®, NALGESIN®, NAPOSIN®, NAPRELAN®, NAPROGESIC®, NAPROSYN®, NAROCIN®, PRONAXEN®, PROXEN®, SOPROXEN®, SYNFLEX®, MOTRIMAX®, AND XENOBID®.

In some embodiments, the analgesic agent is an anticonvulsant, such as pregabalin, lamotrigine, topiramate, oxcarbazepine, tiagabine, levetiracetam, zonisamide, phenytoin, carbamazepine, gabapentin, or ethosuximide.

In some embodiments, the analgesic agent is paracetamol.

In some embodiments, the first dopaminergic agent and/or second dopaminergic agent is a dopamine receptor agonist or a precursor thereof. In some embodiments, the first dopaminergic agent is a D2 agonist, such as pramipexole or carbidopa. In some embodiments, the first dopaminergic agent is carbidopa. In some embodiments, the second dopaminergic agent is a D1 agonist, such as levodopa. In some embodiments, the first and second dopaminergic agents are administered in combination, such as the combination of carbidopa and levodopa (e.g., SINEMET®, ATAMET®, and CARBILEV®).

In some embodiments, the method includes administering an additional therapeutic agent to the subject. The additional therapeutic agent may be, for example, a chemotherapeutic agent. The chemotherapeutic agent may be, e.g., erlotinib (TARCEVA®), bortezomib (VELCADE®), disulfiram, epigallocatechin gallate, salinosporamide A, carfilzomib, 17-AAG (geldanamycin), radicicol, lactate dehydrogenase A (LDH-A), fulvestrant (FASLODEX®), sunitib (SUTENT®), letrozole (FEMARA®), imatinib mesylate (GLEEVEC®), finasunate (VATALANIB®), oxaliplatin (ELOXATIN®), 5-FU (5-fluorouracil), leucovorin, rapamycin (Sirolimus, RAPAMUNE®), lapatinib (TYKERB®), lonafamib (SCH 66336), sorafenib (NEXAVAR®), gefitinib (IRESSA®), AG1478, alkylating agents such as thiotepa and CYTOXAN®, cyclosphosphamide, alkyl sulfonates (e.g., busulfan, improsulfan and piposulfan; aziridines such as benzodopa, carboquone, meturedopa, and uredopa), ethylenimines and methylamelamines (e.g. altretamine, triethylenemelamine, triethylenephosphoramide, triethylenethiophosphoramide and trimethylomelamine), acetogenins (e.g., bullatacin and bullatacinone), a camptothecin (e.g, topotecan and irinotecan), bryostatin, callystatin CC-1065, adozelesin, carzelesin, bizelesin, cryptophycins, adrenocorticosteroids (e.g. prednisone and prednisolone), cyproterone acetate, 5α-reductases, vorinostat, romidepsin, panobinostat, valproic acid, mocetinostat dolastatin; aldesleukin, talc duocarmycin, eleutherobin pancratistatin, sarcodictyin, spongistatin, nitrogen mustards (e.g., chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard), or nitrosoureas (e.g., carmustine, chlorozotocin, fotemustine, lomustine, nimustine, or ranimnustine).

In some embodiments, the additional therapeutic agent is an anti-inflammatory agent, such as acetaminophen, aspirin, celecoxib, cortisone, deracoxib, diflunisal, etoricoxib, fenoprofen, ibuprofen, ketoprofen, lumiracoxib, mefenamic acid, meloxicam, naproxen, parecoxib, phenylbutazone, piroxicam, prednisolone, rofecoxib, sulindac, suprofen, tolmetin, valdecoxib, 4-(4-cyclohexyl-2-methyloxazol-5-yl)-2-fluorobenzenesulfonamide, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-(3,5-difluorophenyl)-3-[4-(methylsulfonyl)phenyl]-2-cyclopenten-1-one, anti-tumor necrosis factor (TN F) agents, anti-interleukin (IL) treatment, or anti-CD20.

In some embodiments, the additional therapeutic agent is an antidepressant, such as alaproclate, citalopram, escitalopram, femoxetine, fluoxetine, fluvoxamine, paroxetine, sertraline, zimelidine, adinazolam, amitriptylinoxide, amineptine, amoxapine, atomoxetine, bupropion, butriptyline, demexiptiline, desmethylclomipramine, desipramine, dimetacrine, dothiepin, doxepin, fluacizine, imipramine, imipramine oxide, iprindole, lofepramine, maprotiline, melitracen, metapramine, norclomipramine, nortriptyline (desmethylamitriptyline), noxiptilin, opipramol, perlapine, pizotyline, propizepine, protriptyline, quinupramine, tianeptine, atomoxetine, reboxetine, tomoxetine, viloxazine, amiflamine, bazinaprine, befloxatone, brofaromine, cimoxatone, clorgyline, iproniazid, isocarboxazid, M-3-PPC, moclobemide, pargyline, phenelzine, selegiline, tranylcypromine, vanoxerine, N-methyl-9-oxo-9H-thioxanthene-3-carboxamide 10,10-dioxide (BW-616U), 1-ethylphenoxathiine 10,10-dioxide (BW-1370U87), 4-chloro-2-[(2R)-2-hydroxy-3-morpholin-4-ylpropyl]-5-phenyl-1,2-oxazol-3-one hydrochloride (i.e., CS-722 or RS-722), (5R)-3-[2-((IS)-3-cyano-1-hydroxypropyl)benzothiazol-6-yl]-5-methoxymethyl-2-oxazolidine (E-2011), harmine, harmaline, N-(2-aminoethyl)-5-(m-fluoro-phenyl)-4-thiazolecarboxamide hydrochloride (RO 41-1049), 4-[(7-hydroxy-6,7-dihydro-5H-cyclopenta[d]pyrimidin-4-yl)amino]benzonitrile (RS-8359), (5R)-3-[6-(cyclopropylmethoxy)naphthalen-2-yl]-5-(methoxymethyl)-1,3-oxazolidin-2-one (T-794), 5-(hydroxymethyl)-3-(3-methylphenyl)-1,3-oxazolidin-2-one (toloxatone), N-propargyl-imipramine hydrochloride (K-Y 1349), N-[2-(2-chlorophenoxy)ethyl]cyclopropanamine (LY-51641), N-[2-(2-iodophenoxy)ethyl]cyclopropanamine hydrochloride (LY-121768), N-[2-(2,4-dichlorophenylthio)ethyl]-N-methyl-prop-2-yn-1-amine (M&B 9303), (E)-beta-fluoromethylene-m-tyrosine (MDL 72394), (E)-beta-fluoromethylene-m-tyramine (MDL 72392), 4-(5-chloro-1-benzofuran-2-yl)-1-methylpiperidine (sercloremine), N-methyl-N-propargyl-quinoline hydrochloride (MO 1671), bazinaprine, befloxatone, brofaromine, cimoxatone, moclobemide, adinazolam, amitriptyline/chlordiazepoxide combination, atipamezole, azamianserin, befuraline, bifemelane, binodaline, bipenamol, caroxazone, cericlamine, cianopramine, clemeprol, clovoxamine, dazepinil, deanol, droxidopa, enefexine, estazolam, etoperidone, fengabine, fezolamine, fluotracen, idazoxan, indalpine, indeloxazine, levoprotiline, lithium, litoxetine, medifoxamine, metralindole, mianserin, minaprine, montirelin, nebracetam, nefopam, nialamide, nomifensine, norfluoxetine, orotirelin, oxaflozane, pinazepam, pirlindole, ritanserin, rolipram, setiptiline, sibutramine, sulbutiamine, sulpiride, teniloxazine, thozalinone, thyroliberin, tiflucarbine, tofenacin, tofisopam, toloxatone, trazodone, veralipride, viqualine, or zometapine.

In some embodiments, the additional therapeutic agent is an antiemetic agent, such as dolasetron, granisetron, ondansetron, tropisetron, palonosetron, mirtazapine, domperidone, olanzapine, droperidol, haloperidol, chlorpromazine, prochlorperazine, alisapride, metoclopramide, aprepitant, casopitant, cyclizine, diphenhydramine, dimenhydrinate, doxylamine, meclizine, promethazine, hydroxyzine, Cannabis, dronabinol, nabilone, benzodiazepine, or hyoscine.

In some embodiments, the additional therapeutic agent is a muscle relaxant, such as benzodiazepine (e.g., diazepam and tetrazepam), nonbenzodiazepines, antispasmodics (e.g., cyclobenzaprine, carisoprodol, chlorzoxazone, meprobamate, methocarbamol, metaxalone, orphenadrine, tizanidine and flupirtine), or antispasticity drugs (e.g., baclofen and dantrolene sodium).

In some embodiments, the analgesic agent, first dopaminergic agent, second dopaminergic agent, and/or additional therapeutic agent is administered to the patient orally, intravenously, intramuscularly, intravitreally, ocularly, intraocularly, itraorbitally, intradermally, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, topically, by inhalation, by injection, by implantation, by infusion, by continuous infusion, by localized perfusion bathing target cells directly, by catheter, by lavage, in creams, or in lipid compositions.

In some embodiments, the pain is acute pain, such as acute pain caused by a sports-related injury, a military injury, other physical trauma, a surgical procedure, cancer, infection, inflammation, or childbirth.

In some embodiments, the subject is at risk of transitioning from experiencing acute pain to experiencing chronic pain. In some embodiments, the method prevents transition of acute pain to chronic pain.

In some embodiments, the pain is chronic pain, such as peripheral neuropathic pain, post-herpetic neuralgia, diabetic neuropathic pain, neuropathic cancer pain, failed back-surgery syndrome, trigeminal neuralgia, phantom limb pain, central neuropathic pain, multiple sclerosis related pain, Parkinson disease-related pain, post-stroke pain, post-traumatic spinal cord injury pain, pain from dementia, musculoskeletal pain, osteoarthritic pain, fibromyalgia syndrome, inflammatory pain, rheumatoid arthritis, endometriosis, migraine, cluster headache, tension headache syndrome, facial pain, headache caused by other diseases, visceral pain, interstitial cystitis, irritable bowel syndrome, chronic pelvic pain syndrome, lower back pain, neck and shoulder pain, burning mouth syndrome, or complex regional pain syndrome. In some embodiments, the chronic pain is lower back pain.

In some embodiments, administration of the analgesic agent, first dopaminergic agent, and second dopaminergic agent commences prior to the onset of the pain, such as within two months prior to the onset of the pain.

In some embodiments, administration of the analgesic agent, first dopaminergic agent, and second dopaminergic agent commences after the onset of the pain, such as within 3 months after the onset of the pain.

In some embodiments, the subject is a mammal, such as a human (e.g., a female). In some embodiments, the subject is a female. In some embodiments, the subject is a male.

In some embodiments, the method further includes performing a brain scan on the subject. The brain scan may reveal, for example, that the subject has reduced connectivity between the subject's nucleus accumbens (NAc) and the subject's medial prefrontal cortex (mPFC) relative to a reference level of NAc-mPFC connectivity. In some embodiments, the subject has been identified as having a reduced connectivity between the subject's NAc and the subject's mPFC relative to a reference level of NAc-mPFC connectivity. In some embodiments, the brain scan is a functional magnetic resonance imaging (fMRI) technique, such as an fMRI technique described herein (see, e.g., Example 1, below). In some embodiments, the reference level is the median level of NAc-mPFC connectivity in a population of patients being tested for likelihood of responding to administration of the analgesic agent, first dopaminergic agent, and/or second dopaminergic agent. The reference level may be, e.g., a z-transformed correlation score of NAc-mPFC connectivity. The subject's NAc-mPFC connectivity may be compared to the reference level, e.g., by assessing the subject's NAc-mPFC z-transformed correlation score. The z-transformed correlation score may be determined, for example, using methods described herein (see, e.g., Example 1, below). In some embodiments, the reference level is a z-transformed correlation score of from about 0.1 to about 0.4 (e.g., 0.1, 0.15, 0.2, 0.25, 0.3, 0.35, or 0.4). In some embodiments, the reference level is a z-transformed correlation score of from about 0.15 to about 0.3 (e.g., 0.15, 0.2, 0.25, or 0.3). In some embodiments, the reference level is a z-transformed correlation score of about 0.2.

In an additional aspect, the disclosure features a pharmaceutical composition containing an analgesic agent in an amount of from about 50 mg to about 500 mg, a first dopaminergic agent in an amount of from about 5 mg to about 75 mg, and a second dopaminergic agent an amount of from about 25 mg to about 300 mg.

In some embodiments, the pharmaceutical composition contains the analgesic agent in an amount of from about 100 mg to about 400 mg (e.g., in an amount of from about 200 mg to about 300 mg, such as in an amount of from about 225 mg to about 275 mg, such as in an amount of about 250 mg).

In some embodiments, the pharmaceutical composition contains the first dopaminergic agent in an amount of from about 7.5 mg to about 25 mg (e.g., in an amount of from about 9 mg to about 20 mg, such as in an amount of from about 10 mg to about 15 mg, such as in an amount of about 12.5 mg). In some embodiments, the pharmaceutical composition contains the first dopaminergic agent in an amount of from about 15 mg to about 50 mg (e.g., in an amount of from about 17 mg to about 35 mg, such as in an amount of from about 20 mg to about 30 mg, such as in an amount of about 25 mg). In some embodiments, the pharmaceutical composition contains the first dopaminergic agent in an amount of from about 25 mg to about 75 mg (e.g., in an amount of from about 45 mg to about 65 mg, such as in an amount of from about 40 mg to about 60 mg, such as in an amount of about 50 mg).

In some embodiments, the pharmaceutical composition contains the second dopaminergic agent in an amount of from about 35 mg to about 100 mg (e.g., in an amount of from about 40 mg to about 75 mg, such as in an amount of from about 45 mg to about 55 mg, such as in an amount of about 50 mg). In some embodiments, the pharmaceutical composition contains the second dopaminergic agent in an amount of from about 50 mg to about 150 mg (e.g., in an amount of from about 75 mg to about 125 mg, such as in an amount of from about 85 mg to about 110 mg, such as in an amount of about 100 mg). In some embodiments, the pharmaceutical composition contains the second dopaminergic agent in an amount of from about 150 mg to about 250 mg (e.g., in an amount of from about 175 mg to about 225 mg, such as in an amount of from about 190 mg to about 210 mg, such as in an amount of about 200 mg).

In some embodiments, the analgesic agent is a non-steroidal anti-inflammatory drug (NSAID), such as naproxen, aceclofenac, acemetacin, acetaminophen, aloxiprin, aspirin, benorilate, bromfenac, celecoxib, deracoxib, diclofenac, diflunisal, ethenzamide, etodolac, etofenamate, etoricoxib, fenbufen, fenoprofen, flufenamic acid, flurbiprofen, lonazolac, lornoxicam, ibuprofen, indomethacin, isoxicam, kebuzone, ketoprofen, ketorolac, licofelone, loxoprofen, lumiracoxib, meclofenamic acid, mefenamic acid, meloxicam, metamizol, mofebutazone, naproxen, nabumetone, niflumic acid, nimesulide, oxaprozin, oxyphenbutazone, parecoxib, phenidone, phenylbutazone, piroxicam, propacetamol, propyphenazone, rofecoxib, salicylamide, sulfinpyrazone, sulindac, suprofen, tiaprofenic acid, tenoxicam, tolmetin, or valdecoxib. In some embodiments, the analgesic agent is naproxen.

In some embodiments, the analgesic agent is an anticonvulsant, such as pregabalin, lamotrigine, topiramate, oxcarbazepine, tiagabine, levetiracetam, zonisamide, phenytoin, carbamazepine, gabapentin, or ethosuximide.

In some embodiments, the analgesic agent is paracetamol.

In some embodiments, the first dopaminergic agent and/or second dopaminergic agent is a dopamine receptor agonist or a precursor thereof. In some embodiments, the first dopaminergic agent is a D2 agonist, such as pramipexole or carbidopa. In some embodiments, the first dopaminergic agent is carbidopa. In some embodiments, the second dopaminergic agent is a D1 agonist, such as levodopa.

In some embodiments, the pharmaceutical composition is formulated for administration orally, intravenously, intramuscularly, intravitreally, ocularly, intraocularly, itraorbitally, intradermally, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, topically, by inhalation, by injection, by implantation, by infusion, by continuous infusion, by localized perfusion bathing target cells directly, by catheter, or by lavage. In some embodiments, the pharmaceutical composition is formulated for oral administration, for example, as a tablet, capsule, gel cap, powder, liquid solution, or liquid suspension. In some embodiments, the pharmaceutical composition is a cream or is a lipid-containing mixture. In some embodiments, the pharmaceutical composition is formulated for sustained release. In some embodiments, the pharmaceutical composition is an implanted device.

In an additional aspect, the disclosure features a kit containing the pharmaceutical composition of any of the above aspects or embodiments of the disclosure. The kit may further contain, for example, a package insert instructing a user of the kit to administer the pharmaceutical composition to a subject (e.g., a mammal, such as a human, for example, a human female or human male) at risk of acute or chronic pain, having acute or chronic pain, or at risk of transitioning from acute pain to chronic pain.

In a further aspect, the disclosure features a kit containing an analgesic agent, a first dopaminergic agent, a second dopaminergic agent, and a package insert instructing a user of the kit to administer the analgesic agent, first dopaminergic agent, and second dopaminergic agent to a subject (e.g., a mammal, such as a human, for example, a human female or human male) in accordance with the method of any of the above aspects or embodiments of the disclosure.

### Definitions

As used herein, the term “about” means ±10% of the recited value.

As used herein, “acute pain” refers to pain that begins suddenly and can be characterized as being short-lived (e.g., twelve weeks or less). It can result from a direct stimuli, such as soft tissue damage (e.g., caused by surgery, dental work, physical trauma, inflammation, or burn) and can be accompanied by a sharp, stinging pain. Typically, acute pain ceases when the stimulus is removed and resolves as the affected tissue(s) heal.

As used herein, “administer” or “administering” refers to a method of giving a dosage of a composition (e.g., a pharmaceutical composition, e.g., a pharmaceutical composition including a combination of a dopaminergic agent and an analgesic agent) to a subject. The compositions utilized in the methods described herein can be administered, for example, intravenously, intramuscularly, intravitreally (e.g., by intravitreal injection), ocularly (e.g., by eye drop, intraocularly, intraorbitally), intradermally, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, orally, topically, by inhalation, by injection, by implantation, by infusion (e.g., by continuous infusion), by localized perfusion bathing target cells directly, by catheter, by lavage, in creams, or in lipid compositions. The compositions utilized in the methods described herein can also be administered systemically or locally. The method of administration can vary depending on various factors (e.g., the compound or composition being administered and the severity of the condition, disease, or disorder being treated).

As used herein, “agent” refers to a substance, compound (e.g., molecule), supramolecular complex, material, or combination or mixture thereof. A compound can be any agent that can be represented by a chemical formula, chemical structure, or sequence. Examples of agents, include, e.g., small molecules, polypeptides, nucleic acids (e.g., RNAi agents, antisense oligonucleotide, aptamers), lipids, polysaccharides, etc. In general, agents can be obtained using any suitable method known in the art. The ordinary skilled artisan will select an appropriate method based, e.g., on the nature of the agent. An agent can be at least partly purified. An agent can be provided as part of a composition, which can contain, e.g., a counter-ion, aqueous or non-aqueous diluent or carrier, buffer, preservative, or other ingredient, in addition to the agent. An agent can also be provided as a salt, ester, hydrate, or solvate. An agent can be cell-permeable, e.g., within the range of typical agents that are taken up by cells and that act intracellularly, e.g., within mammalian cells, to produce a biological effect.

As used herein, “analgesic agent” refers to an agent that acts to inhibit or suppress pain in a subject. The agent may be a drug that acts on the peripheral and/or central nervous system. Exemplary analgesic agents include non-steroidal anti-inflammatory drugs (NSAIDs, e.g., naproxen), anticonvulsants, and paracetamol (i.e., acetaminophen).

As used herein, “anticonvulsant” refers to an agent that reduces the severity or rate of neuronal firing, thereby promoting antiepileptic effects.

As used herein, “antidepressant” refers to a substance that is used in the treatment of mood disorders, such as those characterized by various manic or depressive affects.

As used herein, “anti-inflammatory agent” refers to an agent that functions to reduce inflammation or swelling. This term encompasses small molecule and biologic drugs, such as methotrexate, and antibodies or fragments thereof that interfere with pro-inflammatory associated pathways (e.g., lymphocyte proliferation and inflammatory cytokine release/activity, e.g., anti-05 monoclonal antibodies, anti-TNF antibodies, e.g., etanercept or infliximab). Anti-inflammatory agents also include immunosuppressants, including alkylating agents (e.g., cyclophosphamide), antimetabolites (e.g., azathioprine, methotrexate, leflunomide, and mycophenolate mofetil) and macrolides (e.g., cyclosporine and tacrolimus). Assays to determine the anti-inflammatory activity of a given compound are known in the art.

As used herein, “cancer” refers to or describes the physiological condition in mammals that is typically characterized by unregulated cell growth. Included in this definition are benign and malignant cancers, as well as dormant tumors or micrometastases. Examples of cancer include, but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia, chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

As used herein, “chronic pain” refers to persistent pain that is caused by either 1) a pathological condition, such as infection, arthritis, chronic injury (e.g., sprain), cancer, or neuropathic pain, or 2) an acute stimulus after which neurological signaling is compromised by an aberrant healing process. Such pain can persist long after the inciting event. Chronic pain includes, but is not limited to: peripheral neuropathic pain, (e.g., post-herpetic neuralgia, diabetic neuropathic pain, neuropathic cancer pain, failed back-surgery syndrome, trigeminal neuralgia, and phantom limb pain), central neuropathic pain, (e.g., multiple sclerosis related pain, Parkinson disease related pain, post-stroke pain, post-traumatic spinal cord injury pain, and pain from dementia), musculoskeletal pain (e.g., osteoarthritic pain and fibromyalgia syndrome), inflammatory pain (e.g., rheumatoid arthritis and endometriosis), headache (e.g., migraine, cluster headache, tension headache syndrome, facial pain, headache caused by other diseases), visceral pain (e.g., interstitial cystitis, irritable bowel syndrome, and chronic pelvic pain syndrome), and mixed pain (e.g., lower back pain, neck and shoulder pain, burning mouth syndrome, and complex regional pain syndrome).

As used herein, “chemotherapeutic agent” refers to an anti-cancer drug.

As used herein, “dopaminergic agent” refers to an agent that increases dopamine receptor signaling. Dopaminergic agents include direct agonists of dopamine receptors (including the D1, D2, D3, D4, and D5 receptors), as well as agents that indirectly increases dopaminergic tone. Such agents include dopamine reuptake inhibitors, dopamine releasing agents, and precursors, cofactors, and prodrugs of dopamine or dopaminergic signaling enhancers.

As used herein, “dopamine receptor agonist” refers to an activating ligand of a dopamine receptor. Dopamine receptor agonists can be full or partial agonists and can bind any dopamine receptor type from the D1-like family or the D2-like family.

As used herein, “dopamine releasing compound” refers to an agent that induces the release of dopamine.

As used herein, “dopamine reuptake inhibitor” refers to an agent that functions by blocking the transport of dopamine across physiological compartments (e.g., intracellular vesicles or neuronal synapses). This generally results in the sequestering of dopamine molecules in the vicinity of their receptors and prolonging signaling kinetics.

As used herein, “dose” refers to the quantity of a therapeutic agent, such as an analgesic agent or dopaminergic agent described herein, that is administered to a subject at a given point in time for the treatment or prevention of a condition, such as to reduce pain or prevent the transition of acute pain to chronic pain. A therapeutic agent (e.g., an analgesic agent or dopaminergic agent described herein) may be administered to a subject periodically, for example, by administering multiple doses of the agent at fixed intervals over the course of a treatment period. Alternatively, a subject may be treated by administration of a single dose of a therapeutic agent of interest. In each case, the therapeutic agent may be administered using one or more “unit dosage forms” of the therapeutic agent, a term that refers to a one or more discrete compositions containing a therapeutic agent that collectively constitute a single dose of the agent. For instance, a single dose of 500 mg of a therapeutic agent may be administered using, e.g., two 250 mg unit dosage forms of the therapeutic agent. The unit dosage forms may be, for example, solid unit dosage forms, such as tablets or capsules, among others.

As used herein, “D1 agonist” refers to a drug which induces signaling through the D1-like family of dopamine receptors (i.e., coupled to the G_{sα} protein). The D1-like family includes receptors D1 and D5.

As used herein, “D2 agonist” refers to a drug which induces signaling through the D2-like family of dopamine receptors (i.e., coupled to the G_{iα} protein). The D2-like family includes receptors D2, D3, and D4.

As used herein, an “effective amount” or “effective dose” of an agent (or composition containing such agent) refers to an amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses.

As used herein, “muscle relaxant” refers to an agent that affects skeletal muscle function and decreases smooth muscle tone.

As used herein, “NSAID” refers to an agent that provides analgesic, antipyretic, and anti-inflammatory effects, including non-selective inhibitors of the enzyme cyclooxygenase. The term also includes free acids, free bases, or pharmaceutically acceptable salts thereof.

As used herein, “package insert” refers to instructions customarily included in commercial packages of medicaments that contain information about the indications, usage, dosage, administration, contraindications, other medicaments to be combined with the packaged product, and/or warnings concerning the use of such medicaments.

As used herein, “paracetamol” refers to the compound known as acetaminophen and includes free acids, free bases, and pharmaceutically acceptable salts thereof.

As used herein, “pharmaceutically acceptable carrier or excipient” refers to a carrier (which term encompasses media, diluents, solvents, vehicles, etc.) or excipient that does not significantly interfere with the biological activity or effectiveness of the active ingredient(s) of a composition and that is not excessively toxic to the host at the concentrations at which it is used or administered.

As used herein, the term “prevent” means to reduce the likelihood of developing a condition, or alternatively, to reduce the severity of a subsequently developed condition. A therapeutic agent can be administered to a subject who is at increased risk of developing a disease or condition relative to a member of the general population in order to prevent the development of, or lessen the severity of, the disease or condition. A therapeutic agent can be administered as a prophylactic, e.g., before development of any symptom or manifestation of a disease or condition.

As used herein, “prophylactic treatment” refers to providing medical and/or surgical management to a subject who has not developed a disease or does not show evidence of a disease in order, e.g., to reduce the likelihood that the disease will occur or to reduce the severity of the disease should it occur. The subject can have been identified as being at risk of developing the disease (e.g., at increased risk relative to the general population or as having a risk factor that increases the likelihood of developing the disease).

As used herein, “ratio” of one agent to another agent (e.g., the ratio of dopaminergic agent to analgesic agent) refers to the molar ratio of one compound or compounds to another. In cases describing a ratio of a mixture of D1 agonists and/or D2 agonists to one or more analgesic agents, the ratio is taken as the number of moles of the dopaminergic agent relative to the number of moles of the analgesic agent. In cases having two or more dopaminergic agents (e.g., a D1 agonist and a D2 agonist), the ratio is taken as the total number of moles of the dopaminergic agents relative to the number of moles of the analgesic agent.

As used herein, “reducing pain” means decreasing the severity or duration of a subject's sensation of pain.

As used herein in the context of connectivity between a subject's nucleus accumbens (NAc) and the subject's medial prefrontal cortex (mPFC), the term “reference level” refers to a connectivity threshold below which the subject may be determined to be particularly likely to respond to therapy described herein (e.g., therapy containing an analgesic agent, a first dopaminergic agent (e.g., a D2 agonist), and/or a second dopaminergic agent (e.g., a D1 agonist)). Exemplary NAc-mPFC connectivity reference levels that may be used in conjunction with the compositions and methods of the disclosure include z-transformed correlation scores. A z-transformed correlation score for NAc-mPFC connectivity may be determined, for example, using methods described herein (see, e.g., Example 1, below). Exemplary z-transformed correlation scores that may be used to indicate a subject's likelihood of responding to therapy described herein include z-transformed correlation scores of from about 0.1 to about 0.4 (e.g., 0.1, 0.15, 0.2, 0.25, 0.3, 0.35, or 0.4), among others.

As used herein, a “subject” is a vertebrate (e.g., a mammal, e.g., a human).

As used herein, “therapeutically effective amount” refers to an amount sufficient to produce a desired result, for example, the reduction or elimination of pain in a subject.

As used herein, “treat”, “treating” and similar terms in the context of treating a subject refer to providing medical and/or surgical management of a subject. Treatment can include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment can include reversing, alleviating, reducing severity of, curing, inhibiting the progression of, and/or reducing the likelihood of recurrence of the disease or one or more symptoms or manifestations of the disease. A therapeutic agent can be administered to a subject who has had a disease but no longer shows evidence of the disease (e.g., a subject that is disease-free, but continues to experience pain). The agent can be administered, e.g., to reduce the likelihood of recurrence of evident disease.

### BRIEF DESCRIPTION OF THE FIGURES

The application file contains at least one drawing executed in color. Copies of this patent or patent application with color drawings will be provided by the Office upon request and payment of the necessary fee.

FIG. 1 is a chart showing the design of the clinical trial described in Example 1, below. The chart shows the numbers of subjects who met the criteria for inclusion in, or exclusion from, the study and their distribution among the three study arms. Entry in the treatment groups required a probability of recovery lower than 60%, according to a prediction model based on the baseline brain scans. LDP+NPX, levodopa/carbidopa+naproxen; PLC+NPX, placebo+naproxen; TX, treatment; NoTx, no-treatment; OBS, observation; AE, adverse event; GI, gastrointestinal distress; LTF, lost to follow.

FIG. 2A is a line plot depicting daily average residual back pain intensity trajectories, per study arm: NoTx (green, n=10), LDP+NPX (orange, n=20), and PLC+NPX (purple, n=27) during baseline, treatment and post-treatment observation phases (displayed are 7, 78 and 73 days of ratings for each phase, respectively). There was an interaction between treatment and time (repeated measures mixed model ANOVA, F(314,8478)=1.54, P<0.001), with NoTx residual pain being greater than LDP+NPX (P=0.09) and greater than PLC+NPX (P=0.02, post hoc Dunnet test). Grey background indicates treatment-phase. Shaded areas represent +/−SEM. The table summarizes one-week group-averaged back pain (0-10 scale) at baseline and at three and six months, as a function of treatment type (mean and standard error, SEM, as well as 95% confidence intervals, CI, are shown).

FIG. 2B is a graph showing response rates in the LDP+NPX group (75% of subjects were responders) were not different from PLC+NPX (78% were responders) [Fisher's exact test, P=1.00; with response criteria of 20% or greater reduction in mean pain intensity from baseline to six months, mean pain over one week of ratings]. A trend for superior response rates was seen in the treated population, relative to NoTx (Fisher's exact test, P=0.12).

FIG. 2C is a graph showing residual pain at six months (based on one-week average of ratings) was not statistically different between study arms but showed a trend [Kruskal-Wallis test, P=0.15]. Comparing between NoTx and the two treatments together, treated individuals presented larger improvement in pain intensity (Mann-Whitney U test, P=0.07; medians, quartiles and ranges are shown). (At study closing and before data analyses, based on the original power calculation and total retention, P=0.1 was declared as positive evidence for efficacy). Box plots indicate median, quartiles, and range, while numerals indicate number of subjects. BL, baseline; LDP+NPX, levodopa/carbidopa+naproxen; PLC+NPX, placebo+naproxen; NoTx, no-treatment; Resp, responders; NonResp, non-responders.

FIG. 3A is a line plot depicting average residual daily back pain intensity trajectories for females (F) and males (M) separately, per treatment arm. There was an interaction between time, treatment and gender (F(157,6751)=3.105, P<0.001, repeated measures mixed model ANOVA), with LDP+NPX females presenting lower residual pain than LDP+NPX males (P<0.05), and a tendency against PLC+NPX females (P=0.10), while PLC+NPX males had less residual pain than LDP+NPX males (P=0.06), according to post-hoc Tukey test. Shaded areas represent +/−SEM.

FIG. 3B is a graph demonstrating that, at six months, residual pain (one-week averaged pain ratings relative to baseline) showed a gender by treatment interaction (Two-way ANOVA, F(1,43)=8.16, P=0.007). In the LDP+NPX arm, females presented less residual pain than males at six months (post-hoc Mann-Whitney U test, P<0.001), indicating that LDP+NPX is more efficacious in females. Less residual pain was seen comparing these females against females in the PLC+NPX arm (post hoc Mann-Whitney U test, P=0.06). Contrasting males between treatments, PLC+NPX was more effective than LDP+NPX in males (post hoc Mann-Whitney U test, P=0.06). Box plots show median, quartiles, and ranges; while numerals indicate number of subjects per group.

FIG. 3C shows response rates at both three and six months across different criteria. LDP+NPX showed complete recovery across all thresholds in all females at 6 months, while PLC+NPX was less effective yielding similar response rates in males and females.

FIG. 3D is a graph showing LDP+NPX treated individuals, the average maximum administered dose (given three times/day) was lower in females than in males (two-tailed t-test, P=0.02). Levodopa dose equivalence was calculated according to number of placebo tablets.

FIG. 3E is a table summarizing group-averaged back pain at baseline and at three and six months (0-10 scale), as a function of treatment type and gender (mean and standard error, SEM, as well as 95% confidence intervals, CI are shown). LDP+NPX, levodopa/carbidopa+naproxen; PLC+NPX, placebo+naproxen; TX, treatment; NoTx, no-treatment; SEM, standard error of the mean.

FIG. 4A is a correlation matrix for 19 questionnaire subscale measures. Clustering analysis identified 5 communities, based on data from 116 subjects (all participants available) at the first visit. Communities were labeled according to their component measures: pain intensity, pain sensitivity, pain quality, pain psychology, and negative affect.

FIG. 4B is a pre-treatment network graph displaying interrelations between the five communities, highlighting within- and inter-cluster associations. Edges represent correlations with p<0.01.

FIG. 4C is a network graph showing that, at six months, the network structure was disrupted in treated groups, whilst remaining intact in the NoTx group. Specifically, pain intensity and catastrophizing communities dissociated from the network in the LDP+NPX group.

FIG. 4D is a network graph showing that LDP+NPX treatment decreased the strength of correlations (ΔR) between measures, relative to PLC+NPX and NoTx groups.

FIG. 4E is a network graph demonstrating that network modularity increased in the treatment groups at 6 months, and was largest in LPD+NPX.

FIG. 4F is a graph showing that relative to PLC+NPX, LDP+NPX decreased the strength of network correlations in both males and females. In FIGS. 4D and 4F, error measures were based on permutation testing (10,000 repeated random resampling); error bars are SEMs.

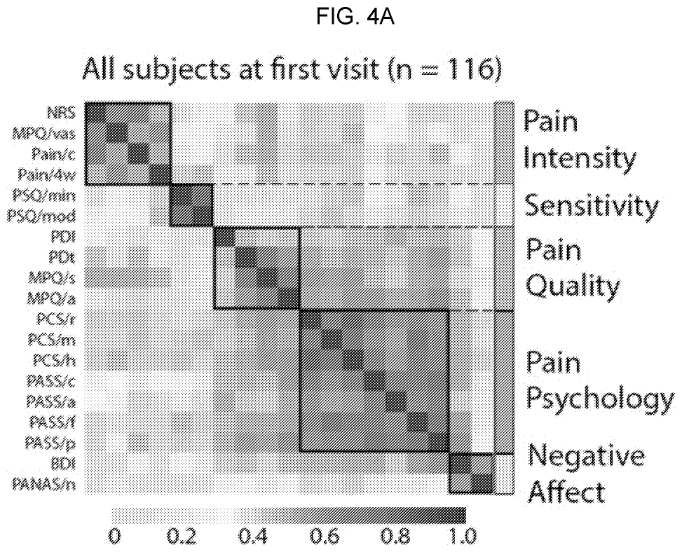

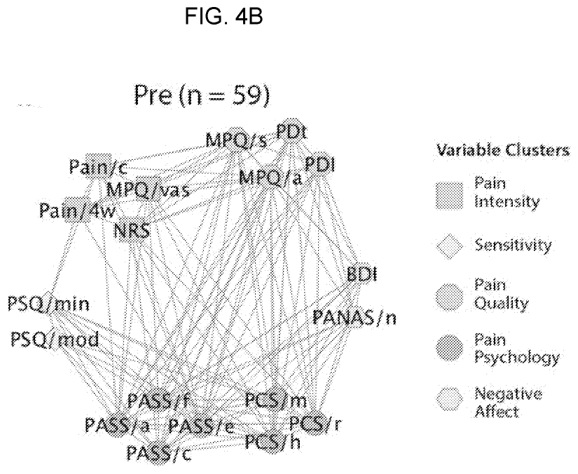

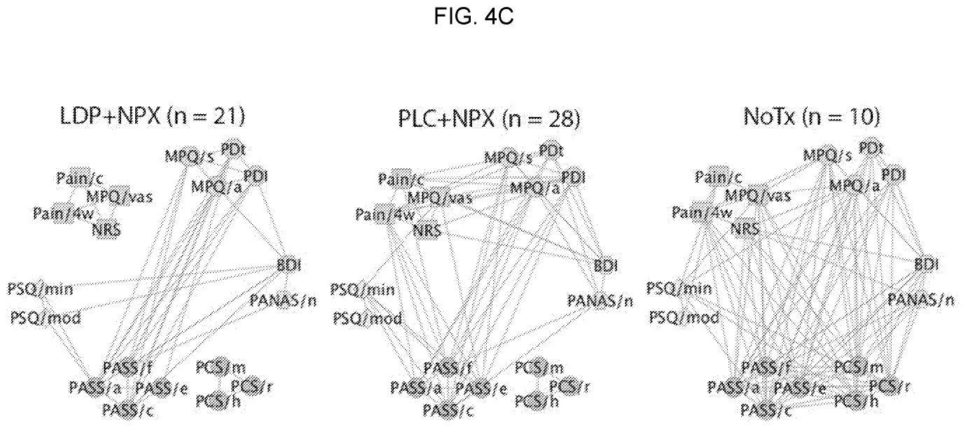

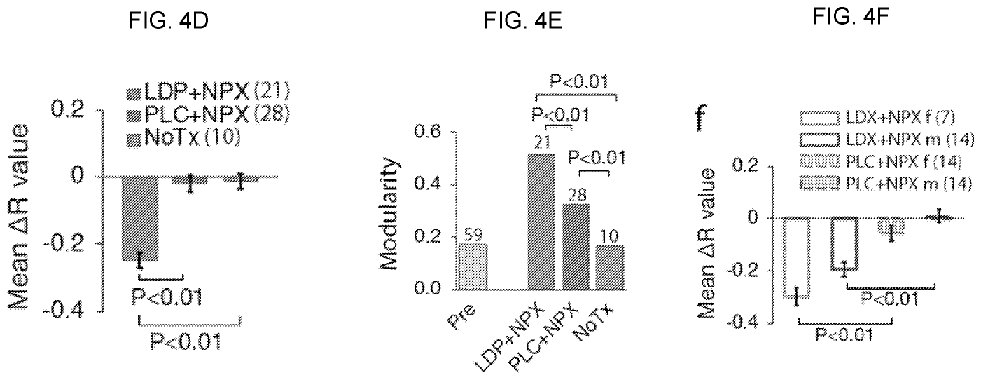

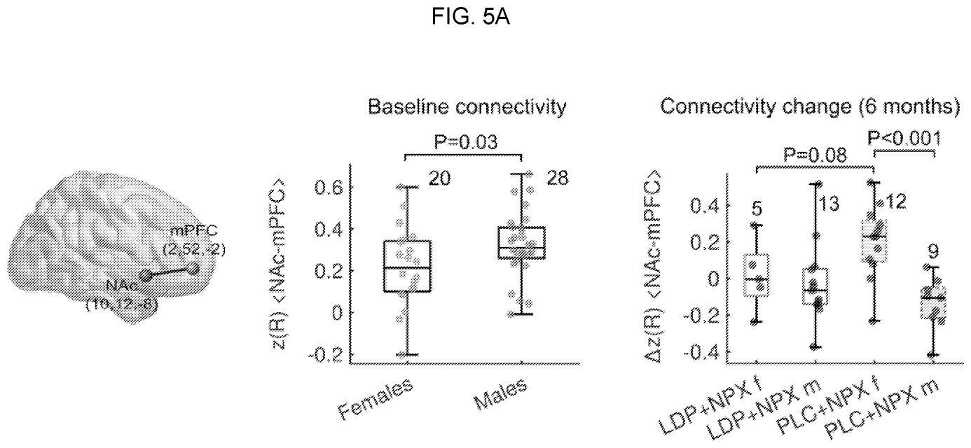

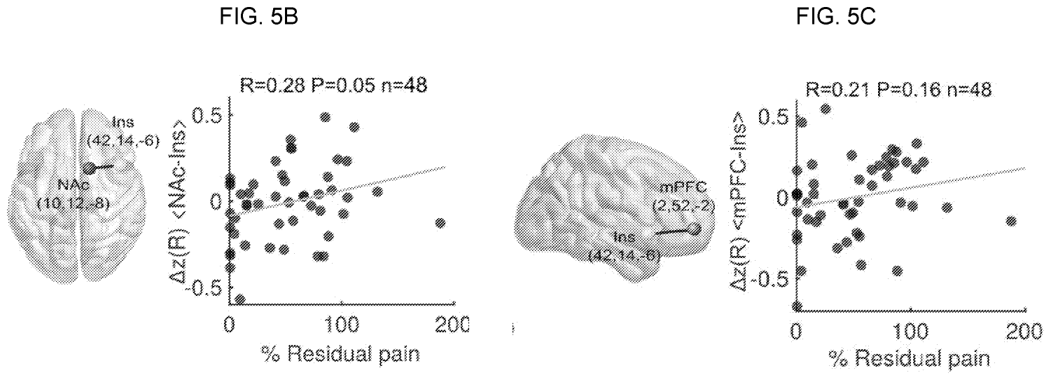

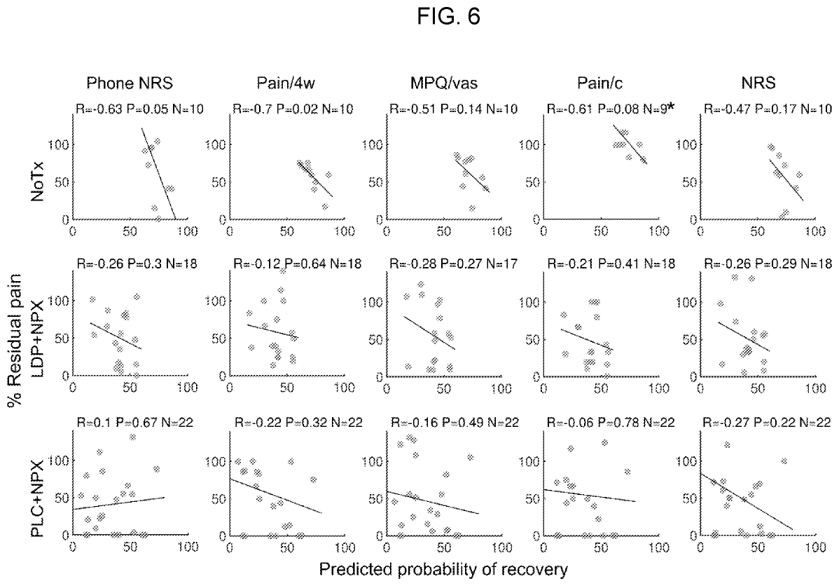

The following abbreviations are used in one or more of FIGS. 4A-4F: LDP+NPX, levodopa/carbidopa+naproxen; PLC+NPX, placebo+naproxen; NoTx, no-treatment. NRS, numerical rating scale; MPQ, McGill Pain Questionnaire—Short Form: visual analog scale (MPQ/vas), sensory (MPQ/s) and affective components of pain (MPQ/a); Pain/c, current pain (PainDETECT); Pain/4w (PainDETECT), average pain over the past four weeks; PSQ, Pain Sensitivity Questionnaire: minor (PSQ/min) and moderate (PSQ/mod); PDI, Pain Disability Index; PDt, PainDETECT; PCS, Pain Catastrophizing Scale: rumination (PCS/r), magnification (PCS/m), and helplessness (PCS/h); PASS, Pain Anxiety Symptoms Scale: cognitive suffering (PASS/c), escape-avoidance behaviors (PASS/e), fear of pain (PASS/f), and physiological symptoms of anxiety (PASS/a); BDI, Beck Depression Inventory; PANAS, Positive and Negative Affect Scale: negative (PANAS/n).

FIG. 5A is a series of graphs showing functional connectivity strength between NAc and mPFC (NAc-mPFC; z-transformed correlation strength, Δz(R)) was weaker in females at baseline (Mann-Whitney U test, P=0.03). At six months, there was an interaction between gender and treatment on the extent of change in NAc-mPFC connectivity strength (Two-way ANOVA, F(1,35)=5.04, P=0.031). NAc-mPFC connectivity change was different between genders in the PLC+NPX group (post hoc Mann-Whitney U test, P<0.001) and between females in each treatment arm (post hoc Mann-Whitney U test, P=0.08).

FIG. 5B is a graph showing changes in NAc-Ins functional connectivity strength positively correlated with residual back pain at six months (Pearson R=0.28, P=0.05).

FIG. 5C is a graph showing change in mPFC-Ins connectivity presented a trend for association with residual pain, after regressing out contribution from age (Pearson R=0.21, P=0.16). LDP+NPX, levodopa/carbidopa+naproxen; PLC+NPX, placebo+naproxen.

FIG. 6 is a series of graphs showing model-based predicted recovery is disrupted by treatment. Scatter plots depict model-based predicted probability of recovery versus residual back pain at 6 months, for each treatment type, and for 5 measures of back pain intensity. There is a consistent negative correlation between predicted probability of recovery and residual pain in the NoTx group, across pain intensity measures, indicating that the predictive model used to stratify patients a priori performed well (top panel). Such associations were absent in both LDP+NPX and PLC+NPX treatment groups (lower two panels), suggesting that treatment successfully disrupted what would have been the natural pain trajectories for these participants.

*There was one Pain/c outlier (with residual pain ˜600%) in the NoTx group who was removed;
Pearson R is displayed. LDP+NPX, levodopa/carbidopa+naproxen; PLC+NPX, placebo+naproxen;
NoTx, no-treatment; NRS, numeric rating scale; MPQ/vas, visual analogue scale of McGill questionnaire;
Pain/c, current pain (PainDETECT); Pain/4w, average pain over the past four weeks (PainDETECT).

### DETAILED DESCRIPTION

The present disclosure features combinations of one or more analgesic agents and one or more dopaminergic agents and associated methods useful for the treatment or prevention of pain. Such combinations potentiate analgesia to 1) alleviate acute pain, 2) prevent the transition from acute pain to chronic pain, and 3) manage chronic pain.

Without being limited by mechanism, the present disclosure is based, in part, on the discovery that induction of neuropathic pain is associated with distinct physiological adaptations of the nucleus accumbens (NAc). One adaptation is an increase in dopamine transporter (DAT) expression, which is accompanied by a drop in extracellular dopamine levels. The other is an increase in the intrinsic excitability of indirect pathway spiny projection neurons (iSPNs) and a reduction in their dendritic surface area and glutamatergic innervation. As iSPNs express D2 dopamine receptors that diminish cellular excitability, peripheral nerve injury can induce an up-regulation of DAT expression in ventral tegmental area (VTA) dopamine neurons, diminishing extracellular dopamine concentration and thus disinhibiting iSPNs. The resulting tonic elevation in iSPN excitability can then trigger homeostatic mechanisms resulting in dendritic shrinkage and the loss of excitatory synaptic input. Thus, dopaminergic agents may work by normalizing iSPN excitability and blunting changes in dendritic morphology and synaptic connectivity, resulting in alleviation of pain.

Additionally, the pain-alleviating effects of dopaminergic agents synergize with analgesic agents, such as naproxen. This synergy could stem from the involvement of both central and peripheral mechanisms in the induction of the chronic state. Specifically, while not being bound by theory, it is possible that analgesic agents alleviate SNI-triggered suppression of VTA activity enough to allow levodopa to normalize NAc dopamine levels.

The present disclosure features methods of treating or preventing pain (e.g., acute pain, chronic pain, or the transition between acute and chronic pain) by administering dopaminergic agents in conjunction with analgesic agents (e.g., as part of the same composition or different compositions, or at the same time or separate times) according to particular dosing regimens. For example, using the compositions and methods of the disclosure, a subject (e.g., a mammal, such as a human, for example, a human female or human male) having acute pain, chronic pain, at risk of having acute pain, or at risk of transitioning from acute pain to chronic pain may be treated by administering to the subject an analgesic agent, a first dopaminergic agent, and a second dopaminergic agent such that:
(i) the analgesic agent is administered to the subject in an amount of from about 50 mg to about 500 mg per dose (e.g., about 100 mg to about 400 mg per dose, about 200 mg to about 300 mg per dose, or about 225 mg to about 275 mg per dose, such as in an amount of about 250 mg per dose);
(ii) the first dopaminergic agent is administered to the subject in an amount of from about 5 mg to about 75 mg per dose (e.g., about 7.5 mg to about 25 mg per dose, such as about 12.5 mg per dose; about 15 mg to about 50 mg per dose, such as about 25 mg per dose; or about 25 mg to about 75 mg per dose, such as about 50 mg per dose); and
(iii) the second dopaminergic agent is administered to the subject in an amount of from about 25 mg to about 300 mg per dose (e.g., about 35 mg to about 100 mg per dose, such as about 50 mg per dose; about 50 mg to about 150 mg per dose, such as about 100 mg per dose; or about 150 mg to about 250 mg per dose, such as about 200 mg per dose).

In some embodiments of the disclosure:
(i) the analgesic agent is administered to the subject in an amount of from about 150 mg to about 1,500 mg per day (e.g., about 200 mg to about 1,200 mg per day, about 500 mg to about 1.00 mg per day, or about 700 mg to about 800 mg per day, such as in an amount of about 750 mg per day);
(ii) the first dopaminergic agent is administered to the subject in an amount of from about 20 mg to about 200 mg per day (e.g., about 25 mg to about 175 mg per day, such as about 37.5 mg per day; about 50 mg to about 100 mg per day, such as about 75 mg per day; or about 75 mg to about 200 mg, such as about 150 mg per day); and
(iii) the second dopaminergic agent is administered to the subject in an amount of from about 100 mg to about 1,000 mg per day (e.g., about 100 mg to about 400 mg per day, such as about 150 mg per day; about 200 mg to about 400 mg per day, such as about 300 mg per day; or about 500 mg to about 700 mg per day, such as about 600 mg per day).

Additionally or alternatively:
(i) the analgesic agent is administered to the subject in an amount of from about 1,000 mg to about 10,000 mg per week;
(ii) the first dopaminergic agent is administered to the subject in an amount of from about 100 mg to about 1,500 mg per week (e.g., about 200 mg to about 500 mg per week, such as about 262.5 mg per week; about 300 mg to about 700 mg per week; such as about 525 mg per week; or about 500 mg to about 1,500 mg per week, such as about 1,050 mg per week); and
(iii) the second dopaminergic agent is administered to the subject in an amount of from about 500 mg to about 5,000 mg per week (e.g., about 750 mg to about 1,300 mg per week, such as about 1,050 mg per week; about 1,500 mg to about 2,500 mg per week, such as about 2,100 mg per week; or about 3,500 mg to about 4,500 mg per week, such as about 4,200 mg per week).

In some embodiments, the analgesic agent is a non-steroidal anti-inflammatory drug (NSAID), such as naproxen, the first dopaminergic agent is a D2 agonist, such as carbidopa, and the second dopaminergic agent is a D1 agonist, such as levodopa. The sections that follow provide a detailed description of additional therapeutic agents that may be used in conjunction with the compositions and methods of the disclosure, as well as a description of conditions that may be treated using such agents and pharmaceutical compositions containing the same.

### Methods of Treatment

The present disclosure features methods of treating a subject experiencing or expecting to experience pain. A subject in need of a treatment for pain can be administered a dopaminergic agent, e.g., a dopamine receptor agonist or precursors thereof (e.g., 2-OH-NPA, 6-Br-APB, 7-OH-DPAT, 8-OH-PBZI, A-412997, A-68930, A-77636, A-86929, ABT-670, ABT-727, amantadine, aplindore, apomorphine, aripiprazole, apomorphine, bifeprunox, BP-897, bromocriptine, cabergoline, carbidopa, carmoxirole, ciladopa, cloazapine, CY-208243, dihydroergocryptine, dihydrexidine, dinapsoline, dinoxyline, dizocilpine, dopamine, doxanthrine, epicriptine, etilevodopa, fenoldopam, flibanserin, ketamine, L-phenylalanine, L-tyrosine, levodopa, lisuride, lysergic acid diethylamide, melevodopa, memantine, metoclopramide, modafinil, pardoprunox, PD-128907, PD-168007, PF-219061, pergolide, phencyclidine, piribedil, pramipexole, propylnorpomorphine, pukateine, quinagolide, quinelorane, quinpirole, RDS-127, rimantadine, Ro10-5824, ropinirole, rotigotine, roxindole, salvinorin A, SKF-23390, SKF-38393, SKF-77434, SKF-81297, SKF-82958, SKF-83959, SKF-89145, sumanirole, terguride, UH-232, umespirone, or WAY-100635).

Dopamine receptor agonists and precursors thereof that preferentially act on D1 receptors include, e.g., levodopa, SKF-38393, SKF-23390, and clozapine. Dopamine receptor agonists and precursors thereof that preferentially act on D2 receptors include pramipexole, bromocriptine, carbidopa, pergolid, lisuride, guinpirole, metoclopramide, and carmoxirole.

Additionally, dopamine reuptake inhibitors (e.g., bupriopion (WELLBUTRIN®), bicifadine, or GBR12909) can be administered as the dopaminergic agent of the disclosure. Other dopamine reuptake inhibitors are known in the art or can be identified by standard pharmacological in-vitro protocols, e.g., as disclosed in Janowsky et al., 1986, J Neurochem, 46 1272-1276. Any other agent or combination that works by directly or indirectly enhancing dopamine signaling can also be used as part of the disclosure (e.g., dopamine releasing compounds, monoamine oxidase inhibitors, e.g., rasagiline or selegiline).

Analgesic agents that can be administered as part of the disclosure include non-steroidal anti-inflammatory drugs (NSAIDs, e.g., COX-1 or COX-2 inhibitors, e.g., naproxen, aceclofenac, acemetacin, acetaminophen, aloxiprin, aspirin, benorilate, bromfenac, celecoxib, deracoxib, diclofenac, diflunisal, ethenzamide, etodolac, etofenamate, etoricoxib, fenbufen, fenoprofen, flufenamic acid, flurbiprofen, lonazolac, lornoxicam, ibuprofen, indomethacin, isoxicam, kebuzone, ketoprofen, ketorolac, licofelone, loxoprofen, lumiracoxib, meclofenamic acid, mefenamic acid, meloxicam, metamizol, mofebutazone, naproxen, nabumetone, niflumic acid, nimesulide, oxaprozin, oxyphenbutazone, parecoxib, phenidone, phenylbutazone, piroxicam, propacetamol, propyphenazone, rofecoxib, salicylamide, sulfinpyrazone, sulindac, suprofen, tiaprofenic acid, tenoxicam, tolmetin, or valdecoxib).

Alternatively, the analgesic agent of the disclosure can be an anticonvulsant (e.g., pregabalin, lamotrigine, topiramate, oxcarbazepine, tiagabine, levetiracetam, zonisamide, phenytoin, carbamazepine, gabapentin, or ethosuximide).

Other analgesic agents, such as paracetamol (i.e., acetaminophen), are known in the art and can be used as part of the methods of the disclosure.

### Acute Pain

A subject (e.g., a female subject) who is likely to experience acute pain (e.g., prior to childbirth, prior to surgical procedures, prior to military operations, prior to athletic activities, or any circumstance in which a subject is at risk of or expecting to experience pain) can be administered one or more doses of a combination of an analgesic agent and one or more dopaminergic agents (e.g., a D2 agonist and a D1 agonist) prior to the onset of the acute pain (e.g., within 1 minute, 5 minutes, 15 minutes, 30 minutes, 1 hour, 2 hours, 4 hours, 6 hours, 12 hours, 18 hours, 1 day, 2 days, 3 days, 4 days, 5 days, 6 days, 1 week, 2 weeks, 3 weeks, 4 weeks, 6 weeks, or 8 weeks prior to the onset of acute pain).

A subject (e.g., a female subject) who is currently experiencing acute pain (e.g., as a result of a sports-related injury, a military injury, other physical trauma, surgical procedure, cancer, infection, inflammation, or any stimuli resulting in an injury sufficient to stimulate a wound-healing response in the subject) can be administered a combination of an analgesic agent and one or more dopaminergic agents (e.g., a D2 agonist and a D1 agonist) shortly after (within, e.g., 1 minute, 5 minutes, 15 minutes, 30 minutes, 1 hour, 2 hours, 4 hours, 6 hours, 12 hours, 18 hours, 1 day, 2 days, 3 days, 4 days, 5 days, 6 days, 1 week, 2 weeks, 3 weeks, 4 weeks, 6 weeks, 8 weeks, 10 weeks, or 3 months) the onset of the acute pain. The administration can begin soon after the onset of acute pain to increase the degree and the likelihood of alleviation of acute pain.

### Chronic Pain

The disclosure also features methods to treat or prevent chronic pain in a subject (e.g., a female subject). Administration of a combination of an analgesic agent and one or more dopaminergic agents (e.g., a D2 agonist and a D1 agonist) for the treatment of chronic pain can occur continuously, as needed, over a period of time (e.g., about four months, five months, six months, seven months, eight months, nine months, ten months, eleven months, twelve months, or longer).

Chronic pain conditions include, e.g., peripheral neuropathic pain, (e.g., post-herpetic neuralgia, diabetic neuropathic pain, neuropathic cancer pain, failed back-surgery syndrome, trigeminal neuralgia, and phantom limb pain), central neuropathic pain, (e.g., multiple sclerosis related pain, Parkinson disease related pain, post-stroke pain, post-traumatic spinal cord injury pain, and pain from dementia), musculoskeletal pain (e.g., osteoarthritic pain and fibromyalgia syndrome), inflammatory pain (e.g., rheumatoid arthritis and endometriosis), headache (e.g., migraine, cluster headache, tension headache syndrome, facial pain, headache caused by other diseases), visceral pain (e.g., interstitial cystitis, irritable bowel syndrome, and chronic pelvic pain syndrome), and mixed pain (e.g., lower back pain, neck and shoulder pain, burning mouth syndrome, and complex regional pain syndrome).

In addition to treating acute pain and chronic pain, methods of the disclosure also feature preventing or delaying the transition from acute pain to chronic pain. A subject (e.g., a female subject) suffering from acute pain can be administered a D2 agonist, a D1 agonist, and an analgesic agent (e.g., a combination of carbidopa, levodopa, and an NSAID, e.g., SINEMET® and naproxen) to prevent the transition to chronic pain. The combination can be administered at a molar ratio of about 1:4:80, respectively (or within a dose range for each component as described herein), in one or more doses.

Conditions in which acute pain can develop into chronic pain are known in the art and include, e.g., nerve damage caused by trauma or disease, which can develop into chronic neuropathic pain. Chronic neuropathic pain conditions include, e.g., peripheral neuropathy, diabetic neuropathy, post-herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Cancer-related acute pain can also be associated with a risk of developing chronic pain. Such conditions include, e.g., tumor-related bone pain, headache, facial pain, visceral pain, post-chemotherapy syndrome, chronic post-surgical syndrome, and post-radiation syndrome. Acute back pain (e.g., resulting from herniated or ruptured intervertebral disks, or abnormalities of the lumbar facet joints, sacroiliac joints, paraspinal muscles or the posterior longitudinal ligament) can also lead to chronic back pain. Infection-related acute pain associated with inflammation can lead to chronic inflammatory pain (e.g., pain associated with arthritis, rheumatoid arthritis, ankylosing spondylitis, inflammatory bowel disease, Crohn's disease, ulcerative colitis, or post-herpetic neuralgia).

The particular combinations of dopaminergic agents and analgesic agents described above can be administered according to the methods described below.

### Routes of Administration

Methods of the disclosure include various dosing regimens. In general, a dopaminergic agent and an analgesic agent can be administered simultaneously (e.g., as part of a single composition) or at different times in separate compositions.

A single dose including both a dopaminergic agent and an analgesic agent can be administered, or multiple doses can be administered. Each of the multiple doses can include an analgesic agent, a dopaminergic agent, or a composition having both a dopaminergic agent and an analgesic agent. Those of ordinary skill in the art will appreciate that appropriate doses in any particular circumstance depend upon the potency of the agent(s) utilized, and can optionally be tailored to the particular recipient. The specific dose level for a subject can depend upon a variety of factors including the activity of the specific agent(s) employed, severity of the disease or disorder, the age, body weight, general health of the subject, etc. Conventional dosage regimens for oral administration of treatments for acute pain are described by Sachs et al., 2005, American Family Physician 1; 71, 913-918.

Methods of the disclosure include any suitable route of administration. The dopaminergic and analgesic agents can be administered, e.g., intravenously, intramuscularly, intravitreally (e.g., by intravitreal injection), ocularly (e.g., by eye drop, intraocularly, intraorbitally), intradermally, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, orally, topically, by inhalation, by injection, by implantation, by infusion (e.g., by continuous infusion), by localized perfusion bathing target cells directly, by catheter, by lavage, in creams, or in lipid compositions.

In some embodiments, for example, an analgesic agent of the disclosure (e.g., naproxen) and one or more dopaminergic agents of the disclosure (e.g., carbidopa and/or levodopa) are orally administered to a subject (e.g., a mammal, such as a human, for example, a human female). The analgesic agent and/or dopaminergic agent(s) may be administered as separate compositions or in admixture with one another. The analgesic agent and/or dopaminergic agent(s), either individually or in admixture with one another, may be in the form, e.g., of a tablet, capsule, gel cap, powder, liquid solution, or liquid suspension.

The disclosure also features formulations of the dopaminergic agents and the analgesic agents that can be administered according to the routes of administration described above.

### Formulations

Dopaminergic agents and analgesic agents can be admixed as part of the same formulation.

Alternatively, they can be separate formulations, which can each be administered separately, or through the same route, as described above. Each agent can be formulated as either a liquid or a solid for any suitable route of administration, such as those described above. For oral administration, agents can be formulated by combining the active compounds with pharmaceutically acceptable carriers well known in the art. Such carriers enable the agents of the disclosure to be formulated as capsules, tablets, pills, dragees, liquids, gels, syrups, slurries, suspensions and the like, for oral ingestion by a subject to be treated. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). Disintegrating agents can be added, such as the cross linked polyvinyl pyrrolidone, agar, or alginic acid or a salt thereof such as sodium alginate. Oral formulations can also be formulated in saline or buffers for neutralizing internal acid conditions or can be administered without any carriers. Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include capsules (e.g., push-fit capsules) made of gelatin, as well as soft, sealed capsules made of gelatin and a plasticizer, such as glycerol or sorbitol. The capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. Formulations for oral delivery can incorporate agents to improve stability in the gastrointestinal tract and/or to enhance absorption.

Preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate, fixed oils, polyethylene glycols, glycerine, propylene glycol or other synthetic solvents, preservatives (e.g., antibacterial agents such as benzyl alcohol or methyl parabens), antioxidants (e.g., ascorbic acid and sodium bisulfite), chelating agents (e.g., ethylenediaminetetraacetic acid), buffers (e.g., acetates, citrates and phosphates), and agents for the adjustment of tonicity (e.g., sodium chloride and dextrose). The pH can be adjusted with acids or bases, such as hydrochloric acid or sodium hydroxide. Such parenteral preparations can be enclosed in ampoules, disposable syringes or multiple dose vials made of glass or plastic.

For administration by inhalation, pharmaceutical compositions can be delivered in the form of an aerosol spray from a pressured container or dispenser which contains a suitable propellant (e.g., carbon dioxide, a fluorocarbon, or a nebulizer). Liquid or dry aerosol (e.g., dry powders, large porous particles, etc.) can be used. The disclosure includes delivery of agents using a nasal spray or other forms of nasal administration (e.g., for delivery to the central nervous system (e.g., the brain)). Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For topical application, the combination of dopaminergic agent and analgesic agent can be formulated in a suitable ointment, lotion, gel, or cream containing the active agents suspended or dissolved in one or more pharmaceutically acceptable carriers. The agents can be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York). The agents can be formulated using any dermatologically acceptable carrier. Exemplary carriers include a solid carrier, such as alumina, clay, microcrystalline cellulose, silica, or talc; and/or a liquid carrier, such as an alcohol, a glycol, or a water-alcohol/glycol blend. The agents can also be administered in liposomal formulations that allow therapeutic agents to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; 8,822,537, and PCT Publication No. WO 96/40061. Examples of other appropriate vehicles are described in U.S. Pat. Nos. 4,877,805, 8,822,537, and EP Publication No. 0586106A1. Suitable vehicles of the disclosure can also include mineral oil, petrolatum, polydecene, stearic acid, isopropyl myristate, polyoxyl 40 stearate, stearyl alcohol, or vegetable oil. Compositions for topical application of analgesic drugs are described in U.S. Pat. No. 5,589,480, included by reference herein.

Formulations can further include a skin penetrating enhancer, such as those described in “Percutaneous Penetration enhancers”, (eds. Smith E W and Maibach H I. CRC Press 1995). Exemplary skin penetrating enhancers include alkyl (N,N-disubstituted amino alkanoate) esters, such as dodecyl 2-(N,N dimethylamino) propionate (DDAIP), which is described in patent U.S. Pat. Nos. 6,083,996 and 6,118,020, which are both incorporated herein by reference; a water-dispersible acid polymer, such as a polyacrylic acid polymer, a carbomer (e.g., Carbopol™ or Carbopol 940P™, available from B. F. Goodrich Company (Akron, Ohio)), copolymers of polyacrylic acid (e.g., Pemulen™ from B. F. Goodrich Company or Polycarbophil™ from A. H. Robbins, Richmond, Va.; a polysaccharide gum, such as agar gum, alginate, carrageenan gum, ghatti gum, karaya gum, kadaya gum, rhamsan gum, xanthan gum, and galactomannan gum (e.g., guar gum, carob gum, and locust bean gum), as well as other gums known in the art (see for instance, Industrial Gums: Polysaccharides & Their Derivatives, Whistler R. L., BeMiller J. N. (eds.), 3rd Ed. Academic Press (1992) and Davidson, R. L., Handbook of Water-Soluble Gums & Resins, McGraw-Hill, Inc., N.Y. (1980)); or combinations thereof.

Dopaminergic agents or analgesic agents can also be formulated for sustained release, such as from an implanted construct or device. In, e.g., orthopedic replacement operations, a sustained release of pain-treating agents into the local or systemic environment is useful to minimize acute pain and the transition to chronic pain. These agents can be formulated as encapsulants within a porous or degradable matrix, such as a biocompatible polymeric matrix. Materials for sustained release of dopaminergic agents from implantable devices are provided by U.S. Pat. No. 8,852,623, incorporated herein by reference. In general, the size, shape, and/or chemistry of a polymeric material, matrix, or formulation can be appropriately selected to result in release in therapeutically useful amounts over a useful time period, in the tissue into the polymeric material, matrix, or formulation is implanted or administered.

A wide variety of biocompatible materials can be utilized as a sustained release carrier to provide the sustained release of dopaminergic agents and analgesic agents, alone or in combination with one or more biologically active agents, as described herein. Any pharmaceutically acceptable biocompatible polymer known to those skilled in the art can be utilized. The biocompatible controlled release material can degrade in vivo within about one year (e.g., within about 2 to 3 months). Specifically, the controlled release material can degrade significantly within one to three months, with at least 50% of the material degrading into non-toxic residues, which are removed by the body, and 100% of the compound of the disclosure being released within a time period within about two weeks (e.g., within about 2 days to about 7 days). A degradable controlled release material can degrade by hydrolysis, either by surface erosion or bulk erosion, so that release is not only sustained but also provides desirable release rates. The pharmacokinetic release profile of these formulations can be first order, zero order, bi- or multi-phasic, to provide the desired reversible local anesthetic effect over the desired time period.

The biodegradable material can be prepared by any method known to those skilled in the art. For example, where the polymeric material includes a copolymer of lactic and glycolic acid, this copolymer can be prepared by the procedure set forth in U.S. Pat. No. 4,293,539, incorporated herein by reference. Alternatively, copolymers of lactic and glycolic acid can be prepared by any other procedure known to those skilled in the art. Other useful polymers include polylactides, polyglycolides, polyanhydrides, polyorthoesters, polycaprolactones, polyphosphazenes, polyphosphoesters, polysaccharides, proteinaceous polymers, soluble derivatives of polysaccharides, soluble derivatives of proteinaceous polymers, polypeptides, polyesters, and polyorthoesters or mixtures and blends of any of these.

Methods for formulating liposomal compositions of analgesic drugs are described in U.S. Pat. No. 5,451,408, included by reference herein, and such methods can be used for formulations of the either the dopaminergic agents, the analgesic agents, or both, in the prescribed ratio.

### Pharmaceutical Compositions

In addition to methods of treating and preventing acute and chronic pain, the disclosure features pharmaceutical compositions having a combination of an analgesic agent and one or more dopaminergic agents (e.g., a D2 agonist and a D2 agonist). For example, pharmaceutical compositions of the disclosure include those containing an analgesic agent (e.g., an NSAID, such as naproxen) in an amount of from about 50 mg to about 500 mg, a first dopaminergic agent (e.g., a D2 agonist in an amount of from about 5 mg to about 75 mg, and a second dopaminergic agent an amount of from about 25 mg to about 300 mg.

In some embodiments, the pharmaceutical composition contains the analgesic agent in an amount of from about 100 mg to about 400 mg. In some embodiments, the pharmaceutical composition contains the analgesic agent in an amount of from about 200 mg to about 300 mg. In some embodiments, the pharmaceutical composition contains the analgesic agent in an amount of from about 225 mg to about 275 mg. In some embodiments, the pharmaceutical composition contains the analgesic agent in an amount of about 250 mg.

In some embodiments, the pharmaceutical composition contains the first dopaminergic agent in an amount of from about 7.5 mg to about 25 mg. In some embodiments, the pharmaceutical composition contains the first dopaminergic agent in an amount of from about 9 mg to about 20 mg. In some embodiments, the pharmaceutical composition contains the first dopaminergic agent in an amount of from about 10 mg to about 15 mg. In some embodiments, the pharmaceutical composition contains the first dopaminergic agent in an amount of about 12.5 mg.

In some embodiments, the pharmaceutical composition contains the first dopaminergic agent in an amount of from about 15 mg to about 50 mg. In some embodiments, the pharmaceutical composition contains the first dopaminergic agent in an amount of from about 17 mg to about 35 mg. In some embodiments, the pharmaceutical composition contains the first dopaminergic agent in an amount of from about 20 mg to about 30 mg. In some embodiments, the pharmaceutical composition contains the first dopaminergic agent in an amount of about 25 mg.

In some embodiments, the pharmaceutical composition contains the first dopaminergic agent in an amount of from about 25 mg to about 75 mg. In some embodiments, the pharmaceutical composition contains the first dopaminergic agent in an amount of from about 45 mg to about 65 mg. In some embodiments, the pharmaceutical composition contains the first dopaminergic agent in an amount of from about 40 mg to about 60 mg. In some embodiments, the pharmaceutical composition contains the first dopaminergic agent in an amount of about 50 mg.

In some embodiments, the pharmaceutical composition contains the second dopaminergic agent in an amount of from about 35 mg to about 100 mg. In some embodiments, the pharmaceutical composition contains the second dopaminergic agent in an amount of from about 40 mg to about 75 mg. In some embodiments, the pharmaceutical composition contains the second dopaminergic agent in an amount of from about 45 mg to about 55 mg. In some embodiments, the pharmaceutical composition contains the second dopaminergic agent in an amount of about 50 mg.

In some embodiments, the pharmaceutical composition contains the second dopaminergic agent in an amount of from about 50 mg to about 150 mg. In some embodiments, the pharmaceutical composition contains the second dopaminergic agent in an amount of from about 75 mg to about 125 mg. In some embodiments, the pharmaceutical composition contains the second dopaminergic agent in an amount of from about 85 mg to about 110 mg. In some embodiments, the pharmaceutical composition contains the second dopaminergic agent in an amount of about 100 mg.

In some embodiments, the pharmaceutical composition contains the second dopaminergic agent in an amount of from about 150 mg to about 250 mg. In some embodiments, the pharmaceutical composition contains the second dopaminergic agent in an amount of from about 175 mg to about 225 mg. In some embodiments, the pharmaceutical composition contains the second dopaminergic agent in an amount of from about 190 mg to about 210 mg. In some embodiments, the pharmaceutical composition contains the second dopaminergic agent in an amount of about 200 mg.

### Kits

The disclosure further provides kits that can have one or more containers (e.g., bottles, blister packs, vials, ampoules) containing unit dosage forms comprising the dopaminergic agents and analgesic agents of the disclosure (e.g., the compositions described above), and, optionally, one or more additional pharmaceutical agents. Each agent (e.g., the dopaminergic agent or the analgesic agent) can be contained in separate containers or in the same container. Associated with such container(s) (e.g., enclosed in a package together with the container) can be a notice in the form prescribed by a governmental agency regulating the manufacture, use or sale of pharmaceutical products (e.g., the US Food & Drug Administration or European Medicines Agency), which reflects approval by the agency of manufacture of use or sale for human administration for treatment of acute or chronic pain. The notice can describe, e.g., doses, routes and/or methods of administration, approved indications, methods of monitoring for therapeutically effective levels, and/or other information of use to a medical practitioner and/or patient.

### Additional Agents

The compositions, methods, and kits of the disclosure can additionally include one or more other therapeutic agents for the prevention or treatment of secondary conditions. Additional agents can be administered at the same time or at a different time (e.g., by the same route of administration or by a different route of administration, or as part of the same or different compositions) as the dopaminergic and analgesic combination. Agents useful in combination with the compositions and methods of the disclosure include antiemetic agents, antidepressants, anti-inflammatory agents, chemotherapeutics, steroids, and muscle relaxants.

Antiemetic agents that can be used as part of the present disclosure include dolasetron, granisetron, ondansetron, tropisetron, palonosetron, mirtazapine, domperidone, olanzapine, droperidol, haloperidol, chlorpromazine, prochlorperazine, alisapride, metoclopramide, aprepitant, casopitant, cyclizine, diphenhydramine, dimenhydrinate, doxylamine, meclizine, promethazine, hydroxyzine, Cannabis, dronabinol, nabilone, benzodiazepine, and hyoscine.

Antidepressants that can be used as part of the disclosure include alaproclate, citalopram, escitalopram, femoxetine, fluoxetine, fluvoxamine, paroxetine, sertraline, zimelidine, adinazolam, amitriptylinoxide, amineptine, amoxapine, atomoxetine, bupropion, butriptyline, demexiptiline, desmethylclomipramine, desipramine, dimetacrine, dothiepin, doxepin, fluacizine, imipramine, imipramine oxide, iprindole, lofepramine, maprotiline, melitracen, metapramine, norclomipramine, nortriptyline (desmethylamitriptyline), noxiptilin, opipramol, perlapine, pizotyline, propizepine, protriptyline, quinupramine, tianeptine, atomoxetine, reboxetine, tomoxetine, viloxazine, amiflamine, bazinaprine, befloxatone, brofaromine, cimoxatone, clorgyline, iproniazid, isocarboxazid, M-3-PPC, moclobemide, pargyline, phenelzine, selegiline, tranylcypromine, vanoxerine, N-methyl-9-oxo-9H-thioxanthene-3-carboxamide 10,10-dioxide (BW-616U), 1-ethylphenoxathiine 10,10-dioxide (BW-1370U87), 4-chloro-2-[(2R)-2-hydroxy-3-morpholin-4-ylpropyl]-5-phenyl-1,2-oxazol-3-one hydrochloride (i.e., CS-722 or RS-722), (5R)-3-[2-((lS)-3-cyano-1-hydroxypropyl)benzothiazol-6-yl]-5-methoxymethyl-2-oxazolidine (E-2011), harmine, harmaline, N-(2-aminoethyl)-5-(m-fluoro-phenyl)-4-thiazolecarboxamide hydrochloride (RO 41-1049), 4-[(7-hydroxy-6,7-dihydro-5H-cyclopenta[d]pyrimidin-4-yl)amino]benzonitrile (RS-8359), (5R)-3-[6-(cyclopropylmethoxy)naphthalen-2-yl]-5-(methoxymethyl)-1,3-oxazolidin-2-one (T-794), 5-(hydroxymethyl)-3-(3-methylphenyl)-1,3-oxazolidin-2-one (toloxatone), N-propargyl-imipramine hydrochloride (K-Y 1349), N-[2-(2-chlorophenoxy)ethyl]cyclopropanamine (LY-51641), N-[2-(2-iodophenoxy)ethyl]cyclopropanamine hydrochloride (LY-121768), N-[2-(2,4-dichlorophenylthio)ethyl]-N-methyl-prop-2-yn-1-amine (M&B 9303), (E)-beta-fluoromethylene-m-tyrosine (MDL 72394), (E)-beta-fluoromethylene-m-tyramine (MDL 72392), 4-(5-chloro-1-benzofuran-2-yl)-1-methylpiperidine (sercloremine), N-methyl-N-propargyl-quinoline hydrochloride (MO 1671), bazinaprine, befloxatone, brofaromine, cimoxatone, moclobemide, adinazolam, amitriptyline/chlordiazepoxide combination, atipamezole, azamianserin, befuraline, bifemelane, binodaline, bipenamol, caroxazone, cericlamine, cianopramine, clemeprol, clovoxamine, dazepinil, deanol, droxidopa, enefexine, estazolam, etoperidone, fengabine, fezolamine, fluotracen, idazoxan, indalpine, indeloxazine, levoprotiline, lithium, litoxetine, medifoxamine, metralindole, mianserin, minaprine, montirelin, nebracetam, nefopam, nialamide, nomifensine, norfluoxetine, orotirelin, oxaflozane, pinazepam, pirlindole, ritanserin, rolipram, setiptiline, sibutramine, sulbutiamine, sulpiride, teniloxazine, thozalinone, thyroliberin, tiflucarbine, tofenacin, tofisopam, toloxatone, trazodone, veralipride, viqualine, and zometapine.

Anti-inflammatory agents that can be used as part of the disclosure include small-molecule drugs such as acetaminophen, aspirin, celecoxib, cortisone, deracoxib, diflunisal, etoricoxib, fenoprofen, ibuprofen, ketoprofen, lumiracoxib, mefenamic acid, meloxicam, naproxen, parecoxib, phenylbutazone, piroxicam, prednisolone, rofecoxib, sulindac, suprofen, tolmetin, valdecoxib, 4-(4-cyclohexyl-2-methyloxazol-5-yl)-2-fluorobenzenesulfonamide, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, and 2-(3,5-difluorophenyl)-3-[4-(methylsulfonyl)phenyl]-2-cyclopenten-1-one.

Anti-inflammatory biologic agents can also be used as part of the disclosure and include, but are not limited to, anti-tumor necrosis factor (TNF) agents (e.g. adalimumab, infliximab, or etanercept), anti-interleukin (IL) treatment (e.g. anti-IL-1α, IL-1β, IL-1 RA), and anti-CD20 (e.g. tiuximab).

Chemotherapeutic agents that can be used as part of the disclosure include erlotinib (TARCEVA®), bortezomib (VELCADE®), disulfiram, epigallocatechin gallate, salinosporamide A, carfilzomib, 17-AAG (geldanamycin), radicicol, lactate dehydrogenase A (LDH-A), fulvestrant (FASLODEX®), sunitib (SUTENT®), letrozole (FEMARA®), imatinib mesylate (GLEEVEC®), finasunate (VATALANIB®), oxaliplatin (ELOXATIN®), 5-FU (5-fluorouracil), leucovorin, rapamycin (Sirolimus, RAPAMUNE®), lapatinib (TYKERB®), lonafamib (SCH 66336), sorafenib (NEXAVAR®), gefitinib (IRESSA®), AG1478, alkylating agents such as thiotepa and CYTOXAN®, cyclosphosphamide, alkyl sulfonates (e.g., busulfan, improsulfan and piposulfan; aziridines such as benzodopa, carboquone, meturedopa, and uredopa), ethylenimines and methylamelamines (e.g. altretamine, triethylenemelamine, triethylenephosphoramide, triethylenethiophosphoramide and trimethylomelamine), acetogenins (e.g., bullatacin and bullatacinone), a camptothecin (e.g, topotecan and irinotecan), bryostatin, callystatin CC-1065, adozelesin, carzelesin, bizelesin, cryptophycins, adrenocorticosteroids (e.g. prednisone and prednisolone), cyproterone acetate, 5α-reductases, vorinostat, romidepsin, panobinostat, valproic acid, mocetinostat dolastatin; aldesleukin, talc duocarmycin, eleutherobin pancratistatin, sarcodictyin, spongistatin, nitrogen mustards (e.g., chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard), and nitrosoureas (e.g., carmustine, chlorozotocin, fotemustine, lomustine, nimustine, or ranimnustine).

Muscle relaxants that can be used as part of the disclosure include benzodiazepines (e.g., diazepam and tetrazepam), nonbenzodiazepines antispasmodics (e.g., cyclobenzaprine, carisoprodol, chlorzoxazone, meprobamate, methocarbamol, metaxalone, orphenadrine, tizanidine and flupirtine), and antispasticity drugs (e.g., baclofen and dantrolene sodium).

### Assessment of Pain

To measure the efficacy of any of the methods, compositions, or kits of the disclosure, a measurement index can be used. Indices that are useful in the methods, compositions, and kits of the disclosure for the measurement of pain include the Pain Descriptor Scale (PDS), the Visual Analog Scale (VAS), the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS) global evaluation score (GES) the Short-Form McGill Pain Questionnaire, the Minnesota Multiphasic Personality Inventory, the Pain Profile and Multidimensional Pain Inventory, the Child Heath Questionnaire, and the Child Assessment Questionnaire.

### EXAMPLES

The following examples are put forth so as to provide those of ordinary skill in the art with a description of how the compositions and methods claimed herein can be performed, made, and evaluated, and are intended to be purely exemplary of the invention and are not intended to limit the scope of the invention.

### Example 1. Analgesic and Dopaminergic Therapy Reduces Acute and Chronic Pain and can Delay or Prevent the Transition from Acute to Chronic Pain

Back pain (BP) is a leading cause of disability worldwide, in urgent need for more efficacious treatments. Preventing transition to chronic back pain (CBP) would rescue patients from years of suffering. Sexually-dimorphic dopaminergic-motivational circuits may be involved in the transition to chronic pain (tCBP). The studies descried in this example test whether carbidopa/levodopa and naproxen (LDP+NPX) could be used to delay or prevent tCBP. Patients were stratified for their risk for tCBP using brain properties. Those identified as low-risk entered a no-treatment arm. The rest were randomized into a double-blind, placebo and naproxen (PLC+NPX) controlled trial of oral LDP+NPX for 12 weeks, and a post-treatment 12-weeks follow-up. 72 participants were enrolled and 59 completed the study. Both treatments resulted in ˜50% pain relief for ˜75% of participants, sustained post-treatment. LDP+NPX was highly effective in females (>80% pain relief), it modified BP personality, and was related to objective brain functional changes. The results show that these early, long-duration, treatments efficiently and persistently block transition to chronic pain, with gender specificity and minimal toxicity.

Chronic pain dramatically diminishes the quality of everyday life in about 20% of the world population, and for 100 million American adults¹. As a large proportion of these patients are treated with opioids, chronic pain remains a primary contributor to the U.S. opiate epidemic². Once established, its reversal becomes very difficult. Available treatments for chronic pain do not cure the condition, and majority of patients remain dissatisfied with their treatments. Thus, there is universal consensus that new, non-opioid, and efficacious treatments, especially for back pain, are urgently needed. Chronic low back pain (CBP) is the most prevalent type of chronic pain, and its brain properties are now well characterized.³⁻⁶ The current trial was limited to subjects with back pain persistent over multiple weeks, that is, subacute back pain (SBP), as such patients have a high probability of developing CBP^{3,4,7} and their pain is within the time window where it may be more malleable and thus reversible with proper treatment. Treatments that could block transition to chronic pain are an ideal preventative strategy to combat back pain, as they spare such patients from years of disability, diminish probability of exposure to opiate treatments, and decrease the associated staggering healthcare cost (estimated to be >$500 billion just in the USA).

Taking advantage of machine learning techniques and recent longitudinal identification of neurobiological markers that predict pain persistence in SBP individuals^{3,4,8}, treatments were targeted to a specific subtype of SBP: those at high-risk of transitioning to CBP (high-risk-SBP). The other subtype, low-risk-SBP, constituted a no-treatment arm (NoTx) and were simply observed for a similar length as those receiving treatment. This strategy was used to validate prediction model, as well as the concept of targeting treatment to appropriate SBP subtypes, thus sparing patients from unnecessary side effects of treatment. Additionally, contrasting outcome differences between NoTx and treatment groups, while accounting for risk differences, provided additional evidence for efficacy. The influence of treatment on the personality profile of back pain individuals was also explored by examining network properties of pain-related questionnaire measures. Given that pain reports are subjective assessments, brain activity properties were also tested to determine whether they could provide objective evidence regarding treatment efficacy.

### Results

A total of 125 SBP participants were screened for eligibility. 72 were eligible and were stratified between high-risk and low-risk of pain persistence. For stratification, a Bayes Naïve model was used that employed two brain imaging derived parameters shown to independently predict pain persistence in SBP patients: regional fractional anisotropy, a white matter property reflecting fiber density and myelination⁸, and the functional connectivity between the right anterior hippocampus with limbic and pain-related regions. Participants that, according to the model, had less than 60% probability of recovering naturally were deemed high-risk-SBP, while the others were classified as low-risk-SBP.

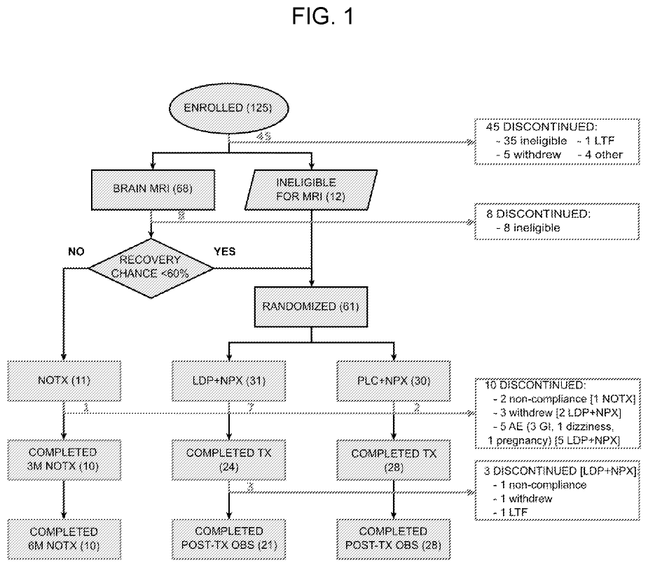

Out of 72 eligible participants, 61 were identified as high-risk (n=61) and were one-to-one randomized between treatment groups: LDP+NPX (n=21 completed) and PLC+NPX (n=28 completed). FIG. 1 describes the trial profile, subject drop-outs and distributions, across the three study arms. After three months of treatment, treatment was ceased but participants were followed for another three months in order to assess long-term effects. Those at low-risk (n=11) were not given any treatment (NoTx group, n=10 completed) and were simply observed for a similar amount of time (6 months) as those randomized to treatment. All subjects who completed the study (n=59) were included in primary analyses. Safety analyses included all participants who received at least one dose of study medication. No notable imbalances in baseline characteristics (age, gender, ethnicity, body mass index, income, education, pain duration) were observed between the three groups. Mean age of the overall population was 47 years, and 41% of participants were females.

### Both Active Treatment Types Result in Sustained Pain Relief

The main outcome measure was a Numerical Rating Scale (NRS),¹⁹ where “0” corresponds to no pain and “10” indicates worst possible pain. On this scale, participants provided ratings of their current pain up to three times a day during study participation (6 months), via a smartphone app (phone NRS).

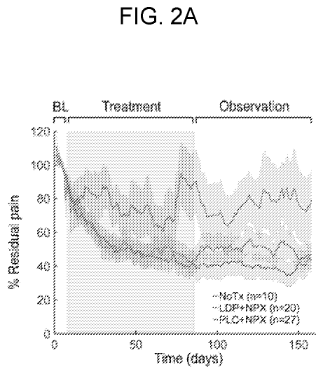

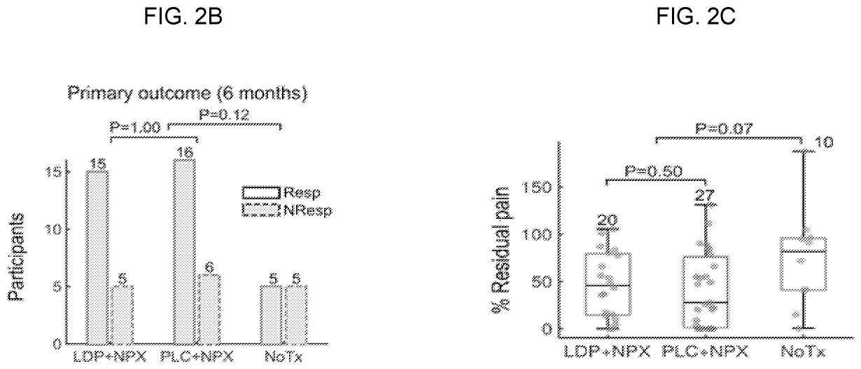

Treatment-related percentage residual pain was computed relative to the average phone-NRS score from the week preceding start of treatment, for every subject and every day, and then averaged across subjects for each treatment type (FIG. 2A). Over the treatment period, a rapid decrease in back pain intensity was observed that, in the NoTx group, stabilizes (with large fluctuations) at around 80% residual pain, while in the LDP+NPX and PLC+NPX groups residual pain continues to decrease for the duration of treatment, reaching a residual pain of about 50%. A mixed-model of time course of pain ratings showed a significant time by group interaction (p<0.001), where NoTx was less effective than both active treatments. Remarkably in all three groups, the observed pain relief persisted after cessation of treatment, over the next 12 weeks.

Contrary to the hypothesis, LDP+NPX did not yield superior pain relief in all patients (i.e., regardless of gender) relative to PLC+NPX at 6 months in high-risk individuals (FIG. 2B). However, both treatment arms (LDP+NPX and PLC+NPX) had about 75% of participants recovering (using an a priori defined criterion of 20% decrease in one-week average phone-NRS from baseline to 6-months; a clinically significant effect size for subacute and chronic back pain relief²⁰), while the NoTx individuals, who were classified as low-risk, had a 50% recovery rate (based on the same a priori criterion). Model expected recovery rate was 60% for NoTx group. Relative to NoTx, both treatments together were associated with larger improvement in back pain intensity (individual subject, and group, one-week average residual pain at 6-months, FIG. 2C). This same pattern is also seen in the table, below, in which one-week average ratings of pain are shown for the three groups at three time points: baseline, 3- and 6-months, and confidence intervals (95% CI) are also reported. In both active treatment arms, but not in NoTx, 95% CI of one-week mean pain shows no overlap in range between baseline and 6-months.

TABLE 1
One-week average pain ratings

| Pain intensity (0-10) | | BL | 3 months | 6 months |
|---|---|---|---|---|
| LDP + NPX | Mean ± SEM | 4.7 ± 0.4 | 2.3 ± 0.4 | 2.3 ± 0.4 |
| (n = 20) | 95% CI | 3.9-5.5 | 1.4-3.2 | 1.4-3.3 |
| PLC + NPX | Mean ± SEM | 5.1 ± 0.3 | 2.4 ± 0.4 | 2.2 ± 0.4 |
| (n = 27) | 95% CI | 4.4-5.8 | 1.6-3.2 | 1.3-3.0 |
| NoTx | Mean ± SEM | 3.6 ± 0.5 | 3.2 ± 0.7 | 2.8 ± 0.7 |
| (n = 10) | 95% CI | 2.4-4.8 | 1.5-4.8 | 1.2-4.3 |

Overall, the NoTx results closely match expected outcome (20% decrease in pain, in 50% of participants) based on the brain-imaging model prediction. Given recent meta-analyses of efficacy of nonsteroidal anti-inflammatories (NSAIDs) in acute and chronic back pain²¹⁻²⁴ and given that active treatments were limited to participants where brain-imaging model categorized them at high-risk, it was expected that PLC+NPX would show less analgesic efficacy than NoTx. Instead, both PLC+NPX and LDP+NPX were observed to show pain relief at a magnitude not observed previously for any drug treatment for acute or chronic back Pain^{21,23,25}. Moreover, in all three groups pain relief was sustained for 3 months after treatment cessation implying a block in the transition to chronic pain.

### Treatment by Gender Interaction

As the brain MCL circuitry is sexually dimorphic^{17,18} and because the transition to chronic pain is critically dependent on properties of MCL and its interaction with the nociceptive drive following inciting events²⁶, it was hypothesized, a priori, that active treatment efficacy should be gender dependent. To this end, active treatment pain ratings were subgrouped by gender, and contrasted outcomes were tested for a treatment by gender interaction.

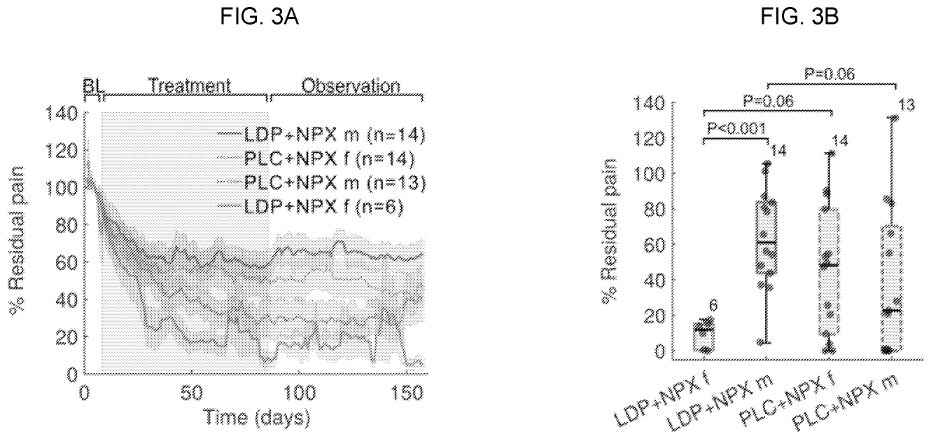

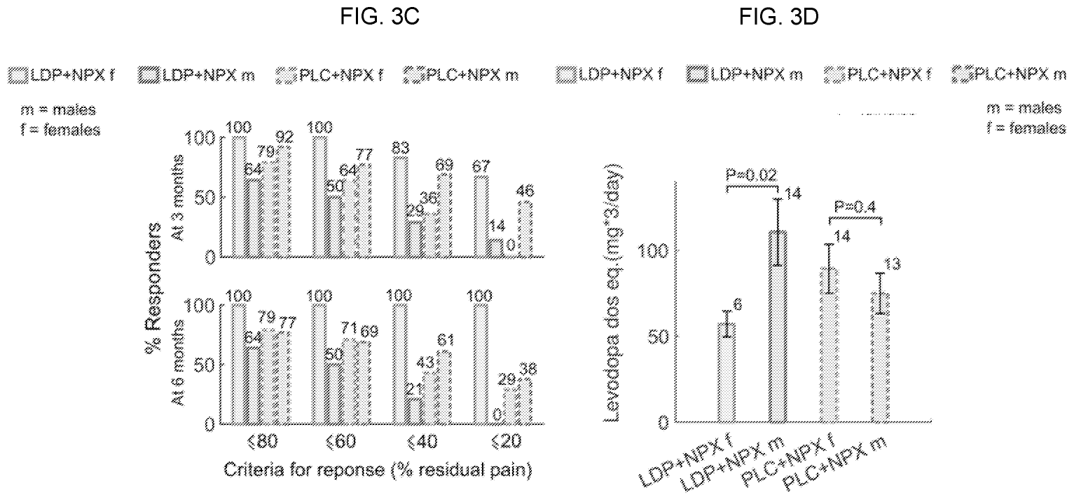

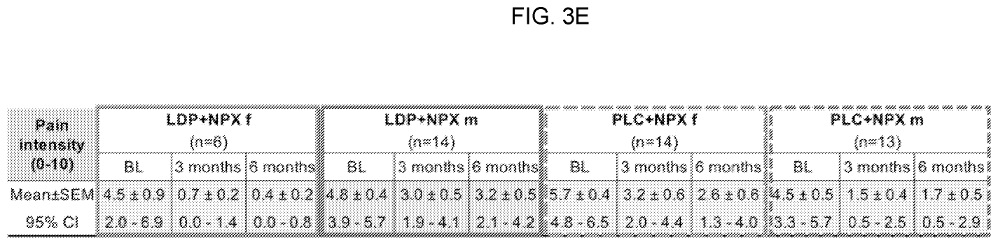

Averaged daily residual pain time course (FIG. 3A) showed a treatment interaction with gender (p<0.001). When residual pain was examined at 6 months (average of one-week, relative to one-week baseline pain), as a function of gender, gender by treatment interaction was again observed (P=0.007, FIG. 3B). In females LDP+NPX treatment was superior to males (p<0.001), and superior to PLC+NPX-treated females (P=0.06). Next, the number of responders to active treatment were explored at 3-months and 6-months when response criterion was varied from 80%-20% residual pain from baseline (FIG. 3C). All females receiving LDP+NPX were responders at six months, even when response criterion was set as high as ≥80% decrease in pain from baseline (residual pain ≤20%), while PLC+NPX was less effective and similar among males and females at all four response criteria. Remarkably, over four response criteria, essentially no change in recovery rates between 3 and 6 months was observed, indicating persistence of pain relief, across treatments and genders.

Given the paucity of information in the field regarding the use of LDP+NPX in human pain management, an appropriate dose for this combination treatment was unknown. Hence the current trial had a dose escalation design, which ranged from 12.5 mg/50 mg three times/day to 50 mg/200 mg carbidopa/levodopa, depending on the observed effects of treatment. Dose titrations were done in a blinded fashion. This design allowed the calculation of the LDP+NPX dose, or equivalent PLC+NPX dose, as a function of gender (FIG. 3D). On average, it was observed that females treated with LDP+NPX were given half of the carbidopa/levodopa dose received by males (P=0.02), despite no gender differences in mean body mass index.

When mean back pain intensity and its range were examined, as a function of gender and treatment type, it was again observed that the largest decrease in back pain is seen in females treated with LDP+NPX (FIG. 3E).

In males, PLC+NPX seems to be more efficient, while LDP seems to interfere with the effect of NPX (FIGS. 3A-3C and 3E). Baseline pain duration, pain intensity, spread of pain on the body, and pain-related questionnaires showed no influence on these outcomes.

Overall, a consistent pattern of LDP+NPX was found to be more effective in females using half the dose of males, and PLC+NPX was more effective in males. Still, in all four sub-groups (gender and treatment type) the observed decrease in pain during treatment persisted for the next three months of observation. The latter again reinforces the notion of a block in the transition to chronic pain.

Change in Back Pain Personality with Treatment

In addition to daily phone ratings, during each visit, participants pain characteristics were assessed by rating their current pain intensity (NRS), and by questionnaires: 1) Pain Sensitivity Questionnaire;²⁷ 2) Pain Disability Index;²⁸ 3) PainDETECT;²⁹ 4) McGill Pain Questionnaire—Short Form;³⁰5) Pain Catastrophizing Scale;³¹ 6) Pain Anxiety Symptoms Scale;³² 7) Beck Depression Inventory;³³ 8) Positive and Negative Affect Scale.³⁴ Rather than examining treatment effects on each of these measures and their subscales, dimensionality reduction techniques were employed, using a clustering algorithm, and a network that summarizes participants' personality profile was constructed. Treatment effects were then examined on this network. This analysis is considered secondary and explores the influence of treatment on global personality properties of back pain individuals.

Baseline back pain personality network was derived from all participants' measures correlation matrix (n=116) (FIG. 4A). Five communities were identified, and were labeled based on member scales: 1) pain intensity; 2) pain sensitivity; 3) pain quality; 4) pain psychology; and 5) negative affect. Following a threshold to only keep correlations with p<0.01, it was observed that these communities were tightly linked with each other at baseline (FIG. 4B). At six months, the network structure was disrupted, depending on treatment type (FIG. 4C). Treatment with LDP+NPX decreased correlation strengths throughout the network both in females and males (FIGS. 4D and 4F), was associated with increased network modularity (FIG. 4E), and dissociated intensity measures from affective and pain-psychological factors (FIG. 4C).

The back pain personality network was thus modified most with LDP+NPX treatment, and in females treated with LDP+NPX, fractionating the pain intensity community from the rest of the network.

### Objective Brain Functional Correlates for Treatment by Gender Interaction and for Pain Relief

Seeking to test objective neurobiological correlates to the subjective reports of treatment effects, functional magnetic resonance images (fMRI) were used, which were collected in most participants at baseline and six months later, to extract relevant brain properties. Candidate markers of treatment efficacy and gender dimorphism were predefined as the extent of information sharing, at rest, between three key regions of interest: the right nucleus accumbens (NAc), medial prefrontal cortex (mPFC) and anterior insula (Ins). Specifically, Ins functional connections with mPFC and NAc (Ins-mPFC, Ins-NAc) were hypothesized to reflect pain intensity, as reported in multiple previous studies,^{8,35,36} while mesolimbic connectivity (mPFC-NAc) was hypothesized to reflect gender specificity.^{17,18} Thus, for each participant, the connectivity strength between each pair of seeds was computed, using previously reported region coordinates^{3,37}, yielding three measures (correlation strengths between Ins, mPFC, and NAc activity at rest) from fMRI scans collected before and after treatment, and these were related to primary outcomes. No additional brain analyses were performed.

These connections reflected distinct aspects related to treatment efficacy (FIGS. 5A-5C). NAc-mPFC functional connectivity presented gender dependency at baseline (P=0.03), and in response to treatment it showed an interaction between gender and treatment (P=0.05, FIG. 5A). Across both active treatments, Ins-NAc (P=0.05) and Ins-mPFC (trend, P=0.16) functional connectivity changes over six months were associated with residual pain at six months (FIGS. 5B and 5C). Therefore, predefined brain network properties yielded objective correlates for treatment efficacy and gender dependence.

### Relationship Between Brain-Derived Risk of Chronic Pain and Treatment Outcomes

The brain anatomy and functional connectivity-based model predicting risk for chronic pain, calculated at time of entry into the study, successfully estimated residual pain six months later in the NoTx arm, based on the main outcome measure (phone NRS, P=0.05), as well as across other pain intensity measures (FIG. 6, top row). These associations were clearly disrupted in both LDP+NPX and PLC+NPX treatment arms, indicating that both treatments successfully disrupted the predicted natural trajectory of pain (FIG. 6, middle and bottom rows). Given that the predictive model was validated in the NoTx group, an expected recovery rate in the treated subjects can be approximated from the model. The model approximates that only less than 25% of the high-risk group (treated with LDP+NPX or PLC+NPX) should have been recovering, while both of these treatments resulted in ˜75% recovering. Thus, these results not only validate the classifier but they also provide additional confidence for treatment efficacy.

### Adverse Events

As the study is the first clinical trial of the combination therapy, documenting adverse events was one of its primary aims. Clinical adverse events were reported by 19 (61.3%) and 17 (56.7%) participants in the LDP+NPX and PLC+NPX groups, respectively, with no statistically significant differences between treatments. Three LDP+NPX participants reported serious adverse events, all of which were judged to be unrelated to the medications used.

### Discussion

The results of this neuroimaging-based trial involving SBP patients demonstrate the ability to delay or prevent transition to chronic pain: 1) a 12-week treatment resulted in pain relief sustained for the next 12 weeks, implying blockade of transition to chronic pain; 2) LDP+NPX treatment was safe, highly effective in females, at a lower dose than that used by males, and fractionated pain intensity components from the back pain personality network; 3) brain functional connectivity reflected gender dimorphism and therapy correlates, providing complimentary objective measures for efficacy; 4) risk for chronic pain measured from brain parameters at time of entry into the study predicted back pain six months later in NoTx, but not in the treated subjects. The data provide objective evidence supporting treatment efficacy.

The trial was designed with the notion that SBP constitutes a highly vulnerable population for developing CBP, who are within a critical time window from pain onset, when associated central reorganizations may be reversible. Therefore, participants were treated for a long-duration and monitored for long-term persistence of efficacy. The results confirm this concept, based on daily ratings of back pain, profiling of SBP personality, and through objective brain biomarkers and brain-derived model for risk of chronic pain. Thus, the approach seems to reset the back pain into a new and persistently improved level for the next three months, suggesting that the extent of reorganization accompanying treatment should persevere in the longer term.

In SBP females, LDP+NPX treatment was more efficacious than any therapies currently available for acute, subacute, or chronic BP^{38,39}. At present, there are no sex-specific treatments for pain. Instead studies tend to either only include males or pool both sexes together for analyses of treatment outcomes⁴⁰. Short-term NSAIDs are currently recommended for acute, subacute (first line), and chronic BP (first line)⁴¹, yet effects on pain intensity relative to placebo are small: −8.4% (95% CI −12.7 to −4.1) for acute BP, −3.3% (95% CI −5.3 to −1.3) for persistent BP, (weighted mean differences in pain reduction, with 95% confidence intervals, for 0-100 point scale at short-term follow-up)^{39,41}. Relative to baseline, CBP patients show mean reduction of −14.3% (95% CI −16.0 to −12.6) following NSAID treatment²¹.

Antidepressants and opioids are also used to treat persistent BP; however, these are not more effective than NSAIDs^{21,25,42}. For non-specific back pain, meta-analysis show that the analgesic effect across different types of treatments are small, with 47% of patients having <10 points effect (on 0-100 point scale), and only 15% having point estimates of >20 points relief²³. In contrast, it was observed that 50% of NoTx achieve >20% relief, and 75% of LDP+NPX or PLC+NPX groups achieve >20% relief. All SBP females in the present study had at least 80% improvement in their pain intensity after taking LDP+NPX, while males had better outcomes taking PLC+NPX (38% of males showing at least 80% improvement with PLC+NPX, while no males taking LDP+NPX had 80% relief). The large difference in efficacy seen between female and male SBP is surprising. This is the first evidence of gender specific, highly effective treatment for back pain, especially with exceptional outcomes for females.

The study data also support the use of network analysis to profile personality dimensions of back pain patients. Fundamental characteristics of the network topology are fractionated to different extents with each treatment type, and with gender dependence. This analysis provides a global overview of the pain personality of the subjects studied and shows that treatment modulates fundamental properties of the network, with much less effect on specific questionnaire unitary outcomes.

As LDP+NPX has not been previously studied in the context of human pain, it was important to carefully document associated adverse events. No adverse effects specifically linked to LDP use were observed. Instead, non-intended events commonly reported were related to NSAIDs. Current American and European guidelines for management of CBP recommend limited time of use NSAIDs due to adverse effects:²¹ shortest duration possible and up to three months, respectively. In meta-analysis mean duration for treating BP with NSAIDs was 5-7 days.²² Here, participants were treated for three months. Both PLC+NPX and LDP+NPX treated patients had unperturbed vital signs and acceptable adverse-event profiles, which were similar to those reported for NSAIDs in general,⁴³ with the additional LDP having minimal or no evidence of increased side effects. Therefore, obtained results generalize to a whole category of analgesics, namely NSAIDs, where a 12-week standard analgesic dose of any NSAIDs use in males, and standard dose of NSAIDs plus pediatric doses of levodopa/carbidopa in females should block transition to chronic pain, at least for SBP with back pain of duration <20 weeks, and decrease intensity of back pain by about 50%. Importantly, no evidence of the back pain duration moderating treatment outcome was observed; thus, such would be expected to be efficacious in patients with longer durations of persistence of back pain, such as in CBP, as well as sub-acute pain conditions, especially other musculoskeletal pains.

Collectively, the results presented here establish that early long-duration treatment of back pain with naproxen and its combination with levodopa/carbidopa are viable treatments, with persistent efficacy of at least three months after treatment cessation. It should be noted that dense monitoring of pain (daily ratings over ˜6 months) combined with a blinded trial and with objective brain correlates of primary outcomes, render the results highly unlikely to be due to a consequence of random effects. Instead, the results should be considered as objective evidence for treatment efficacy and persistence that are unprecedented in magnitude, despite the limited number of subjects studied. Additionally, the approach provides confidence in the outcome of treatments in connection with subjective conditions, like pain. Therefore, the data demonstrate that transition to chronic pain can be blocked. Long-duration low-dose dopamine combination treatment with anti-inflammatory analgesics in women should be considered clinically to cure/prevent chronic pain. Long-term early treatment with anti-inflammatory analgesics alone should be considered sufficient to prevent chronic pain in men.

### Methods

This 24-week double-blind parallel randomized controlled trial was conducted at Northwestern University (Chicago, USA). Protocol and informed consent form were approved by Northwestern University IRB as well as NIDCR/NIH. All enrolled participants provided written informed consent. Safety and trial oversight were monitored by an independent data safety monitoring board and a clinical research organization, with NIDCR/NIH oversight. The trial was registered on ClinicalTrials.gov, under registry NCT01951105.

### Participants

Individuals with a recent onset of low back pain, SBP, were recruited through online social media, local advertising, and via Northwestern Medicine Enterprise Data Warehouse. Criteria for enrollment included: history of low back pain with duration between 4-20 weeks, with signs and symptoms of radiculopathy, average reported pain intensity greater than 4 (on an NRS scale from 0 to 10) on the week before baseline assessments and the week preceding treatment start. Subjects were excluded if they reported other chronic painful conditions, systemic disease, history of head injury, psychiatric diseases or more than mild depression (score >19, according to Beck's Depression Inventory³³).

### Inclusion Criteria:

Male or female, over the age of 18 years, (no racial/ethnic restrictions);
Must have a history of low back pain for a minimum of 4 weeks and a maximum of 12 weeks with signs and symptoms of radiculopathy: positive straight leg raising test with dermatomal radiation and/or myotomal weakness and/or reflex asymmetry; pain must radiate into buttock or below; Must have a high risk phenotype for chronification of back pain (evaluated at baseline T1-MRI, DTI-MRI, and fMRI scans);
Must have an average pain score over a 5 day period (average of ˜15 measures on smartphone app) immediately preceding the baseline visit of 5 (on a 0-10 NRS) at the baseline visit;
Must be willing to read and able to understand instructions as well as protocols;
Must be in generally stable health; and
Must sign an informed consent document after complete explanation of the study documenting that they understand the purpose of the study, procedures to be undertaken, possible benefits, potential risks, and are willing to participate.

### Exclusion Criteria:

Previous (distinct) episodes of back pain onset (more than 3 distinct episodes of back pain lasting for a total of more than 4 weeks) in the previous year;
Evidence of rheumatoid arthritis, ankylosing spondylitis, acute vertebral fractures, chronic spinal stenosis, prior back surgery and history of tumor of the spine;
Low back pain associated with any systemic signs or symptoms, e.g., fever, chills;
Other comorbid chronic pain conditions such as fibromyalgia or neuropathic pain secondary to diabetes or post-herpes zoster;
Chronic neurologic conditions, including Parkinson's disease, Alzheimer's disease, and other conditions associated with dementia;
Significant other medical disease such as congestive heart failure, coronary or peripheral vascular disease, chronic obstructive lung disease, or malignancy;
Diabetes Type I or Type II;
History of glaucoma or narrow angle glaucoma;
Presence of undiagnosed skin lesions or history of melanoma;
Presence of severe cardiovascular or pulmonary disease, bronchial asthma, renal, hepatic or endocrine disease;
History of myocardial infarction with residual cardiac arrhythmia;
History of gastrointestinal bleeding or peptic ulcer;
Diagnosis of current depression (assessed via BDI, total >28 are excluded) or psychiatric disorder requiring treatment, or such a diagnosis in the previous 6 months;
Use of therapeutic doses of antidepressant medications (i.e., tricyclic antidepressants, SSRIs, SNRIs; low doses used only in the evening for sleep will be allowed if dose is not changed);
Current use of recreational drugs or recent history of alcohol abuse (pattern of drinking having social, financial or physical consequences) or drug abuse;
Any change in medication for back pain in the last 30 days;
High dose opioid prophylaxis, defined as >50 mg morphine equivalent/day;
Use of MAOIs, currently or within the past 2 weeks;
Prior use of Levodopa;
Use of any of the following drugs: bromocryptine, linezolid, metoclopramide, phenothiazines, promethazine/codeine, isoniazid, rifampin, pyrazinamide;
Oral iron supplementation;
Contraindications to use of study product, based on any of the following:
   Hypersensitivity to Carbidopa/Levodopa or other constituents of the Carbidopa/Levodopa capsules;
   Hypersensitivity to lactose or other constituents of the placebo capsules;
   Hypersensitivity to Naproxen or other constituents of the Naproxen capsules;
   Hypersensitivity to Acetaminophen or other constituents of the Acetaminophen tablets;
Currently taking Levodopa or dopaminergic drugs;
Involvement in litigation regarding their back pain or having a disability claim or receiving workman's compensation or seeking either as a result of their low back pain;
In the judgment of the investigator, unable or unwilling to follow protocol and instructions;
Intra-axial implants (e.g. spinal cord stimulators or pumps);
All exclusion criteria for MR safety: any metallic implants, pacemaker, brain or skull abnormalities, tattoos on large body parts, and claustrophobia;
Pregnancy or inability to use an effective method of birth control in sexually active men and women while taking the study drug and for one week thereafter. Barrier contraceptives (condoms or diaphragm) with spermicide, intrauterine devices (IUD's), hormonal contraceptives, oral contraceptive pills, surgical sterilization, and complete abstinence are examples of effective methods of contraception;
Following laboratory abnormalities: liver function tests (SGOT/SGPT) greater than twice the upper limit of normal; unexplained anemia (Hgb <9 g/dL); evidence of renal insufficiency (creatinine >upper limit of normal) or any other abnormality that the principal investigator feels puts the subject at risk during the study;
History of chronic opioid use for pain management; or
Any medical condition that in the investigator's judgment may prevent the individual from completing the study or put the individual at undue risk.

### Trial Design

Eligible subjects first underwent a brain imaging session and, using predefined brain-derived parameters, were stratified into high and low-risk subgroups for transitioning to CBP. Those in the low-risk category, who had 60% or higher probability of recovering naturally, entered the NoTx arm; the rest, who had less than 60% probability of recovering naturally, were randomized between a placebo-control arm, and a pharmacological treatment arm. A flexible dose escalation procedure was used and both researchers and participants were blinded to treatment type. Study medications were tapered off at week 12, and participants continued to be evaluated for another 12 weeks. At final visit, participants underwent a second brain scan.

The design was setup to evaluate safety, efficacy and gender dependence of the combination of carbidopa/levodopa (12.5 mg/50 mg-25 mg/100 mg-50 mg/200 mg, dose escalation based on response) plus naproxen 250 mg (LDP+NPX) administered three times a day, compared to placebo plus naproxen 250 mg (PLC+NPX) administered three times a day.

### Modeling Risk for Chronic Pain and Stratifying Participants

A naïve Bayes classifier was used to estimate probability of recovery from back pain for each participant, before they entered the treatment phase. The classifier was trained using data from a previous longitudinal study in SBP and employed two brain markers to predict risk for CBP. These were: the mean fractional anisotropy (FA) of white matter regions of interest that together predicted persistence of low-back pain at one year in previous work,⁸ and the number of functional connectivity links (degree count, De) between the right anterior hippocampus, and limbic and pain-related areas. Every participant eligible for MRI underwent an initial brain scan, and brain derived FA and De measures were entered into the equation above to identify probability for recovery. This measure was used to stratify participants into treatment and no-treatment arms. Participants who could not be scanned (n=12) were assumed to belong to the persisting category (as expected incidence rate for persisting category was ˜80%) and were entered into the treatment arm.

### Randomization and Masking

Eligible individuals were assigned to treatment arms (LDP+NPX, PLC+NPX) based on a computer-generated permuted block randomization scheme, with block size randomly varying and an allocation ratio of 1:1. Allocation concealment was ensured by utilization of sequentially numbered containers. An unblinded individual from Northwestern University Clinical and Translational Sciences (NUCATS), with no other role in the study, was responsible for assuring proper medication assignment to each container. The randomization code was maintained by NUCATS and was available in cases of emergency or clinical situations in which knowing the treatment allocation would make a difference in the safety or management of a subject. In such a circumstance, the allocation assignment was made available after consultation with the site investigator and the principal investigator. This procedure was implemented for one participant who developed a serious adverse event during the treatment period. At study conclusion, after database lock, the randomization code was made available during data analysis.

Participants received 2 capsules on a three times a day (TID) schedule and one capsule once a day (QD). TID medications included one capsule of naproxen and one capsule of either placebo or some dose of carbidopa/levodopa. Omeprazole 40 g was taken QD in the morning, as a preventive measure against gastric adverse effects of naproxen. In order to assure that participants took their study medication as designed, the naproxen and omeprazole were placed in a separate colored capsule from the carbidopa/levodopa. Each colored capsule was dispensed in separate containers and participants were asked to take one capsule from each container. Acetaminophen was available as a rescue medication and all participants were given equal amounts.

### Procedures

The study consisted of eight visits spread over ˜28 weeks, which included an initial screening/monitoring period (˜2 weeks), followed by a treatment period (12 weeks) and a post-treatment observational period (12 weeks). Safety and adverse events were assessed at each clinical visit, including vital signs. At each visit, researchers reminded participants to take their medication according to instructions. Safety was also assessed through blood screenings at baseline, while blood pressure, pulse and temperature were assessed at all study visits.

### Dose Escalation Protocol

Treatment with carbidopa/levodopa was titrated up to 12.5 mg/50 mg three times/day over one week and then continued at that level for 4 weeks. If by the end of the initial 4-week period the participant “responded” [had >20% decrease in pain intensity from the average of all phone NRS ratings collected at baseline (between visit 1 and visit 3) to the average of all phone NRS ratings during those 5 weeks], the participant was maintained on that dose for the duration of the treatment period (12 weeks total). If there was no response, the carbidopa/levodopa dose was increased to 25 mg/100 mg three times/day for the following 4 weeks, at which time the pain status was re-evaluated (based on the average of all phone NRS rating during those four weeks, against baseline average). Again, if a response occurred, that dose was maintained in a blinded manner for the following 4 weeks of treatment; if not, further dose-titration carbidopa/levodopa occurred to 50 mg/200 mg three times/day for the final 4 weeks. When a participant experienced an adverse event at higher doses, the participant was given the next lower dose that s/he was able to tolerate and then maintained on that dose for the remainder of the 12-week dosing period. Naproxen dose (250 mg three times/day) remained constant for all participants throughout the study, except it was not given during the tapering down at the end of the study.

Monitoring Pain Intensity with Phone App

A main outcome measure was a Numerical Rating Scale (NRS),¹⁹ where “0” corresponds to no pain and “10” indicates worst possible pain. Participants were instructed to provide such ratings three times a day, for at least one week prior to randomization, and throughout study participation, via a smartphone app (phone NRS), which contained the following instructions: “Please rate your current level of pain”.

### Phone NRS Preprocessing

Percentage residual pain at three and six months were computed based on the average phone-NRS score from the week preceding treatment and the final week of treatment or post-treatment. Thus, 100% residual pain reflected no change from baseline levels; while 0% residual pain reflected complete recovery. Participants were deemed responders if residual pain at six months was 80% or less (representing 20% pain relief), as defined a priori per protocol.

Specifically, when an individual had more than three ratings in a given day, only three were kept: the first, the last and one rating in between. Next, the average of these ratings was computed for each day. For missing daily ratings, the mean from nearest neighbors were used to replace the missing value. Two subjects who completed the study were not included in the phone NRS analyses due to non-compliance in providing phone ratings.

For each subject, percentage residual back pain (% Residual painᵢ) at a given day i was computed as:

% Residual painᵢ=100−100*[(pain_{b}−painᵢ)/pain_{b}],

where pain_{b} is the baseline pain intensity, pre-defined as the average daily phone-NRS during the week preceding start of treatment, and painᵢ is the mean phone-NRS at day i. Thus, 100% residual pain reflects no change from baseline levels; while 0% residual pain reflects complete recovery. Primary outcome measure was pre-defined as the average phone-NRS residual pain during the final week of the study (six months from start of treatment, i.e, three months after end of treatment). Participants were considered responders if they had 80% or less residual pain at six months.

Additionally, outcomes were examined at three months (that is, at end of treatment), which was defined as the average phone-NRS residual pain during the final week of treatment. For analyses of pain intensity trajectories, included were 7 days preceding treatment, 78 days during treatment, and 73 days post-treatment.

### Other Outcomes

In addition to phone ratings, during each visit, participants rated their current pain intensity (NRS), and were administered self-report questionnaires:

1) Pain Sensitivity Questionnaire (PSQ),²⁷ a 17-item instrument used to assess individual pain sensitivity—it is based on pain intensity ratings of hypothetical situations, which includes various modalities (heat, cold, pressure, pinprick) and measures (pain threshold, intensity ratings); It can be split into two subscales: one consisting of items referring to mildly painful situations (minor, PSQ/min), and one consisting of the items referring to moderately painful situations (moderate, PSQ/mod);
2) Pain Disability Index (PDI),²⁸ an assessment of physical impairment in relation to pain;
3) PainDETECT,²⁹ a 12-item assessment of neuropathic-like symptoms. PDt includes questions of current pain intensity (Pain/c) and subjective report of average pain intensity over the past 4-weeks (Pain/4w);
4) McGill Pain Questionnaire-Short Form(sf-MPQ),³⁰ a well-validated measure assessing both sensory and affective components of pain (MPQ/s and MPQ/a). It also includes a visual analog scale (VAS) of pain;
5) Pain Catastrophizing Scale (PCS),³¹ is a 5-point instrument to assess 13 thoughts or feelings on past pain experience. PCS yields three sub-scale scores assessing rumination (PCS/r), magnification (PCS/m), and helplessness (PCS/h);
6) Pain Anxiety Symptoms Scale (PASS),³² measures fear and anxiety responses specific to pain. PASS consists of four aspects of pain-related anxiety: cognitive suffering (PASS/c), escape-avoidance behaviors (PASS/e), fear of pain (PASS/f), and physiological symptoms of anxiety (PASS/a);
7) Beck Depression Inventory (BDI),³³ is a 21-item instrument for measuring the severity of depression;
8) Positive and Negative Affect Scale (PANAS),³⁴ has two mood scales, one measuring positive affect and the other measuring negative affect (PANAS/n). Each scale is rated on a 5-point, 10-item scale. These measures were considered secondary and were used to construct back pain personality profile and examine treatment effects globally.

Adverse events (AEs) that occurred during treatment and those that occurred after treatment were differentiated. However, to account for potential late onset AEs or those related to withdrawal of medication, AEs occurring during a certain window after treatment were considered to be “during treatment”. Explicitly, as defined, AEs were accounted for as “occurring during the treatment period” if they were observed during the interval from the first dose of study drug to 28 days after the last dose of study drug, or end of study participation, whichever occurred first.

Information on gender, race and ethnicity were obtained using a standard National Institute of Health demographics form, which did not explicitly differentiate between gender and biological sex. Here, the word gender was used, as that was the word used on the self-report form completed by participants. Candidate brain markers of treatment efficacy and gender dimorphism were predefined as the extent of information sharing, at rest, between three key regions of interest: the right nucleus accumbens, medial prefrontal cortex and insula. Specifically, insula connectivity was hypothesized to reflect pain intensity, as found in previous study,⁸ while mesolimbic connectivity was hypothesized to reflect gender specificity.^{17,18} Thus, for each participant, the connectivity strength between each pair of seeds was computed, yielding three measures from fMRI scans collected before and after treatment, and these were related to the primary outcomes. No additional brain analyses were performed.

### Brain Imaging Acquisition

Data were acquired on a clinical 3T Siemens Magnetom Prisma whole body scanner equipped with a receive-only 64 channel head/neck coil. At both baseline and 6 months after randomization, participants underwent a high-resolution anatomical scan (T1-weighted MRI), one resting and one spontaneous pain rating functional MRI (fMRI), and a white matter fractional anisotropy (FA) assessment scan (Diffusion-weighted MRI). The entire MRI scan consisted of approximately 35 minutes of actual image acquisition, plus around 25 minutes for setting patients comfortably in the scanner, attempting to minimize their back pain, and for re-acquiring images in case of excessive motion.

T1-weighted MRI acquisition: High-resolution T1-weighted brain images were collected using integrated parallel imaging techniques (PAT; GRAPPA). The acquisition parameters were: voxel size: 1×1×1 mm³, TR=2.3 s, TE=2.40 ms, TI=900 ms, flip angle=9°, 176 sagittal slices and acceleration factor=2. Phase encoding direction was anterior to posterior, and the duration of acquisition was ˜5 min.

Resting-state fMRI acquisition: Blood oxygen level-dependent (BOLD) T2*-weighted multiband accelerated echo-planar images were acquired at rest. Acquisition parameters were as follows: TR=555 ms, TE=22 ms, flip angle=47°, 64 slices acquired with interleaved ordering, FOV=208 mm, matrix size=96×104, spatial resolution: 2×2×2 mm³, acceleration factor: 8. Phase encoding direction was posterior to anterior. Slices were acquired with ascending order to preserve the continuity of connections. The acquisition lasted ˜10 min, during which 1110 volumes were collected. Participants were instructed to keep their eyes open and to remain as still as possible during acquisition.

Spontaneous pain rating fMRI acquisition: Identical acquisition parameters and duration to resting-state fMRI were used to obtain BOLD T2*-weighted images while participants used a finger-spanning device to continuously rate and log the rate of their spontaneous back pain on a scale of 0-100, in the absence of external stimulation.⁴⁴ Participants were instructed to keep their eyes open and to remain as still as possible during the scan.

Diffusion-weighted MRI acquisition: Multi-slice echo planar imaging with multiband excitation and multiple receivers was used to obtain diffusion-weighted images along 30 and 64 evenly spaced and non-collinear directions, with weighting factors of 700 s/mm² and 2000 s/mm², respectively. Two non-weighted volumes were acquired, each at the beginning of each scan. Voxel size=2×2×2 mm³, TR=3.5 s, TE=92 ms, FOV=230 mm, matrix size=116, number of slices=72, flip angle=90°, multiband factor=3, acquisition time ˜7 min.

### Brain Imaging Processing (for Baseline Model Parameters)

For each subject, MRI data was processed within less than a week from the baseline acquisition and before the randomization visit, in order to extract brain parameters for patient stratification. The quality of each image modality was assessed for excessive motion and poor signal to noise ratio before preprocessing, using a robust quality control pipeline.⁴⁵

Spontaneous pain rating fMRI: right hippocampus connections with limbic and “pain” regions: fMRI volumes were preprocessed using tools within the FMRIB Software Library 5.0.9 (FSL), and MATLAB R2016a. After removing the first 120 volumes of each spontaneous pain rating functional dataset for magnetic field stabilization, skull extraction using BET and slice-time correction were performed. fsl_motion_outliers was used to remove the effect of intermediate to large motion. Next, the remaining 990 volumes were filtered with a band-pass temporal filter (using Butterworth; 0.008 Hz<f<0.1 Hz) and a non-linear spatial filter (using SUSAN; FWHM=5 mm). Finally, nine vectors were regressed out, including the six parameters obtained from intra-modal motion correction using MCFLIRT, global signal (averaged over all voxels of the brain, over the 990 volumes), white matter signal (eroded white matter mask), and cerebrospinal fluid signal (eroded ventricular mask). These nine vectors were filtered with the Butterworth band-pass filter before being regressed from the time series to avoid recontamination.

The cleaned images were then linearly registered to the MNI template using FLIRT and down-sampled to 6×6×6 mm³ voxel size. A C-based, in-house program called “ABLM” (Apkarian Brain Linkage Map), previously described by Baria and colleagues,⁴⁶ was used to compute the mean count of functional connectivity links (degree) between voxels within a predefined mask within the right hippocampus and limbic and “pain”-related regions. Link density threshold for calculation of degree was set to the top 10% of connections.

Diffusion MRI: mean FA: Preprocessing of diffusion-weighted images was performed using eddy current to correct for eddy current-induced distortions and subject movement. Using DTIFIT the diffusion tensor was estimated in each voxel by linear regression and FA maps were derived. Following this, each FA map was non-linearly registered to the FMRIB58_FA template. Next, the mean FA value of a group of voxels that was previously identified as having predictive value for pain chronification⁸ was extracted and this value was used in the Naïve Bayes model for stratifying participants between high and low risk SBP (further details about the model are given below).

### Brain Imaging Processing (for Post-Hoc Analyses)

For post-hoc analyses of brain function, a preprocessing pipeline, which was optimized for longitudinal investigation, was used. After removing the first 20 volumes (corresponding to 11s) of each functional dataset for magnetic field stabilization, data were subjected to skull extraction using BET, slice-time correction, motion correction with MCFLIRT, intensity normalization and high-pass temporal filtering (0.008 Hz). Motion censoring was next performed by detecting volumes with framewise displacement (a measure of how much the head changed position from one frame to the next) larger than 1 mm, DVARS (indexes the rate of change of BOLD signal across the entire brain at each frame of data) with z-score larger than 2.3, or BOLD signal z-score >2.3, and removing their adjacent volumes (−5−4 −3−2 −1 0 1 2 3 4 5).⁴⁷ Signals from three vectors were regressed out, including global signal (averaged signal over all voxels of the brain, over the 1090 volumes), white matter signal (extracted from an eroded white matter mask), and cerebrospinal fluid signal (extracted from an eroded ventricular mask). Finally, the cleaned time series were band-pass filtered (0.008-0.1 Hz) to keep the low-frequency fluctuations of interest.

Functional image registration was optimized for longitudinal analysis by utilizing a two-step approach that minimizes within-subject variability. First, for each subject functional images from scan 1 and scan 2 were registered to each other using forward and backward halfway linear transformations (FLIRT, 6 degrees of freedom). This process allows optimal within subject alignment, with minimal displacement artifacts for both scans.⁴⁸ Second, the midway template was registered to MNI152 space using non-linear registration (FNIRT). In order to minimize warping effects, all non-linear registrations were constrained by affine registration as described by Smith et al.⁴⁹ Final images were spatially smoothed with a Gaussian kernel (2 mm sigma). Registered data were visually inspected to ensure optimal alignment.

### Statistical Considerations and Analyses

The predefined primary outcome measure for efficacy, as a function of treatment type and relative to gender, was the percentage of participants recovering from back pain. Fisher's exact test was used. Additionally, for analysis of pain intensity trajectories repeated-measures ANOVA was employed. The secondary endpoint was to test validity of the model used for stratifying SBP, and also examine changes in brain connectivity with treatment. Exploratory analyses were conducted to examine treatment effects on pain-related questionnaire sub-scales, using dimensionality reduction methods and network analyses. Binary outcomes are reported based on absolute and relative descriptive statistics, consistent with CONSORT guidelines.⁵⁰ All statistical tests were two-sided.

Power analysis for primary outcomes: Because the primary dependent variable was binary (recovery from back pain or not), and to have sufficient power to detect treatment effects, power analysis based on comparison of independent proportions was conducted. A previous study in SBP shows that in high-risk SBP ˜90% persisted with back pain if treated with standard of care (mostly occasional use of anti-inflammatories).³ Thus, for power calculations, it was assumed that 90% of participants that receive placebo and naproxen would have persisting pain at study end. Calculations in G*Power 3.1.9.2 indicated that significant differences with 80% power would be detected if the probability of pain persistence is reduced to 67% or smaller (Type I error rate assumed at 0.05, two-tailed, n=50 per group). A two-tailed comparison was assumed, even though there was no a priori reason for the placebo plus naproxen to outperform the test drug. It was planned to enroll 126 participants, accounting for attrition and for the no-treatment group.

A second aim was to determine if there is a gender by treatment interaction effect. Specifically, the influence of gender on response to active treatment was tested with carbidopa/levodopa and naproxen. Assuming no gender effect on the placebo and naproxen treatment response rate, what remains is the active treatment arm. Calculations in G*Power 3.1.9.2 indicated that an odds ratio greater than 2.7 (2.5-3.0) would be detected with 80% power with an overall sample size of 50 (assuming n=˜25 for each gender). A two-tailed comparison was assumed because there was no a priori reason for a given sex to respond better to the active drug. Again, as a supplemental predefined analysis strategy, it was assumed that the analyses would be strengthen by removing variance due to nuisance covariates and using longitudinal modeling. Thus, total planned participants needed to increase by an additional 60 subjects, making the total planned recruitment 186. However, due to financial constraints, only 125 participants could be recruited, and as a result it was determined (prior to any data analysis) that the primary outcome would be considered positive if comparison outcome passed the less stringent criterion of p<0.1, rather than p<0.05.

Model testing: In order to test the validity of the classifier which stratified participants between high and low risk of pain persistence, the actual responses of the NoTx group were examined at 6 months from entry in the study against the predicted response from the model. To strengthen the validation, in addition to testing the actual response based on the primary outcome measure (Phone NRS), actual versus predicted recovery was also checked with other measures of pain intensity.

Identifying brain markers for treatment effects: Three regions of interest were defined a-priori: the right nucleus accumbens (NAc), medial prefrontal cortex (mPFC) and right anterior insula (aIns). These were based on previous studies showing that: 1. Connectivity strength between mPFC and aIns encode pain intensity across pain conditions,³⁷ and 2. NAc-mPFC connectivity strength is causally associated with transition from subacute to chronic back pain.

Mean time series representative of these regions were extracted from 10 mm radius spheres around previously reported MNI coordinates: NAc (10, 12, −8), mPFC (2, 52, −2) and aIns (42, 14, −6).^{3,37} The Pearson correlation coefficient (R) was computed, 3 numbers/brain, between BOLD signals from each pair of regions to represent their connectivity strength (extent of information sharing).

Questionnaire based exploratory endpoints: Given the large number of additional questionnaires used to assess participants' treatment response, dimension reduction methods and network analysis methods were used to summarize these outcomes. These results are deemed exploratory in nature.

Missing questionnaire data: In case of missing within-questionnaire items, these were replaced with the average of the remaining within-questionnaire scores, provided that the number of unanswered questions was less than 30% of all items in each scale. If more than 30% of the items were blank, subjects were excluded from statistical tests relevant to the given scale.

Clustering analysis: To resolve multicollinearity, dimensionality-reduction techniques were applied to pain-related questionnaires at baseline (visit 1). Variable clustering under VARCLUS algorithm (SAS Institute Inc. 2017e) computed Pearson correlation-based similarity between all 19 included measures and assigned these measures to five clusters. VARCLUS is an iterative algorithm that calculates a determination coefficient (R²) between each variable and a cluster (own R²), of which the variable is a member, and R² between each variable and the next most similar cluster (next R²). The (1−R²) Ratio is defined as (1−own R²)/(1−next R²), where a ratio greater than 1 means that the next closest cluster is more similar than the current cluster.

Network analysis: Each of the N=19 pain-related questionnaire measures was used as a node. Based on this division, an undirected connectivity matrix B=R^{N×N} representing all subjects who completed study participation at baseline was constructed. First, the Pearson correlation (R) coefficient was calculated for each of the possible pairs of nodes to generate a connectivity matrix. Next, this matrix was thresholded to only keep significant connections (P<0.01) and binarized yielding an undirected adjacency matrix. This process was repeated for each group (LDP+NPX, PLC+NPX and NoTx) separately at baseline, and at six months to investigate long-lasting treatment effects. For network visualization, Cytoscape, an open source software⁵¹, was used.

To characterize the structure of these adjacency matrices, modularity was computed using the Brain Connectivity Toolbox (BCT).⁵² A module can be defined as a set of nodes that are densely connected among themselves but sparsely connected to other parts of the network. Modularity quantifies how well-defined these densely connected sets of nodes are within the network. From the different modularity algorithms available, the fast and accurate multi-iterative generalization of the Louvain method was selected for use, provided within BCT. Using this technique, a single unitary value between 0 and 1, representing the modularity of each network, was obtained, where values closer to 1 indicate highly structured systems and values closer to 0 represent random networks. In order to deal with potential modularity degeneracy, modularity was computed over 100 repetitions and the average of these iterations was used as the final modularity measure.

Global network structure was further investigated by examining changes in connectivity on the non-binarized network from baseline to six months and averaging these changes over the entire network to obtain mean ΔR.

Group differences: Modularity and mean ΔR values were compared between groups using a permutation test. First, for each pair-wise measure, the difference between the two groups was calculated as the actual group difference. Second, the lowest common number of subjects was identified and the combined pool of the two conditions was resampled into two new groups. The values of these two resampled groups were calculated next. This process was repeated 10,000 times to generate a null distribution of the mean difference between the groups. The p-value of the actual group difference was calculated as the chance probability from the mean in the null distribution.

Software. Analyses were done using MATLAB 2016a, JMP Pro version 13.2 (SAS Institute, Cary, N.C.) and SPSS version 25. Safety analyses included all participants who received at least one dose of the study drug.

### REFERENCES

1. Murray, C. J. & Lopez, A. D. Measuring the global burden of disease. N Engl J Med 369, 448-457 (2013).
2. Jalal, H., et al. Changing dynamics of the drug overdose epidemic in the United States from 1979 through 2016. Science 361(2018).
3. Baliki, M. N., et al. Corticostriatal functional connectivity predicts transition to chronic back pain. Nat Neurosci 15, 1117-1119 (2012).
4. Vachon-Presseau, E., et al. Corticolimbic anatomical characteristics predetermine risk for chronic pain. Brain 139, 1958-1970 (2016).
5. Hashmi, J. A., et al. Shape shifting pain: chronification of back pain shifts brain representation from nociceptive to emotional circuits. Brain 136, 2751-2768 (2013).
6. Baliki, M. N., Baria, A. T. & Apkarian, A. V. The cortical rhythms of chronic back pain. J Neurosci 31, 13981-13990 (2011).
7. Costa Lda, C., et al. Prognosis for patients with chronic low back pain: inception cohort study. BMJ 339, b3829 (2009).
8. Mansour, A. R., et al. Brain white matter structural properties predict transition to chronic pain. Pain 154, 2160-2168 (2013).
9. Petre, B., et al. Smoking increases risk of pain chronification through shared corticostriatal circuitry. Hum Brain Mapp 36, 683-694 (2015).
10. Lee, M., et al. Activation of corticostriatal circuitry relieves chronic neuropathic pain. J Neurosci 35, 5247-5259 (2015).
11. Ren, W., et al. The indirect pathway of the nucleus accumbens shell amplifies neuropathic pain. Nat Neurosci 19, 220-222 (2016).
12. Chang, P. C., et al. Role of nucleus accumbens in neuropathic pain: linked multi-scale evidence in the rat transitioning to neuropathic pain. Pain 155, 1128-1139 (2014).
13. Vachon-Presseau, E., et al. The Emotional Brain as a Predictor and Amplifier of Chronic Pain. J Dent Res 95, 605-612 (2016).
14. Mogil, J. S. Sex differences in pain and pain inhibition: multiple explanations of a controversial phenomenon. Nat Rev Neurosci 13, 859-866 (2012).
15. Fillingim, R. B., Doleys, D. M., Edwards, R. R. & Lowery, D. Clinical characteristics of chronic back pain as a function of gender and oral opioid use. Spine 28, 143-150 (2003).
16. Meucci, R. D., Fassa, A. G. & Faria, N. M. Prevalence of chronic low back pain: systematic review. Rev Saude Publica 49(2015).
17. Koscik, T., Bechara, A. & Tranel, D. Sex-related functional asymmetry in the limbic brain. Neuropsychopharmacology 35, 340-341 (2010).
18. Munro, C. A., et al. Sex differences in striatal dopamine release in healthy adults. Biol Psychiatry 59, 966-974 (2006).
19. Farrar, J. T., Young, J. P., Jr., LaMoreaux, L., Werth, J. L. & Poole, R. M. Clinical importance of changes in chronic pain intensity measured on an 11-point numerical pain rating scale. Pain 94, 149-158 (2001).
20. Ostelo, R. W. & de Vet, H. C. Clinically important outcomes in low back pain. Best Pract Res Clin Rheumatol 19, 593-607 (2005).
21. Enthoven, W. T. M., Roelofs, P. D. & Koes, B. W. NSAIDs for Chronic Low Back Pain. JAMA 317, 2327-2328 (2017).
22. Machado, G. C., et al. Non-steroidal anti-inflammatory drugs for spinal pain: a systematic review and meta-analysis. Ann Rheum Dis 76, 1269-1278 (2017).
23. Machado, L. A., Kamper, S. J., Herbert, R. D., Maher, C. G. & McAuley, J. H. Analgesic effects of treatments for non-specific low back pain: a meta-analysis of placebo-controlled randomized trials. Rheumatology (Oxford) 48, 520-527 (2009).
24. Rasmussen-Barr, E., et al. Non-steroidal anti-inflammatory drugs for sciatica. Cochrane Database Syst Rev 10, CD012382 (2016).
25. Berthelot, J. M., Darrieutort-Lafitte, C., Le Goff, B. & Maugars, Y. Strong opioids for noncancer pain due to musculoskeletal diseases: Not more effective than acetaminophen or NSAIDs. Joint Bone Spine 82, 397-401 (2015).
26. Baliki, M. N. & Apkarian, A. V. Nociception, Pain, Negative Moods, and Behavior Selection. Neuron 87, 474-491 (2015).
27. Ruscheweyh, R., et al. Validation of the pain sensitivity questionnaire in chronic pain patients. Pain 153, 1210-1218 (2012).
28. Tait, R. C., Pollard, C. A., Margolis, R. B., Duckro, P. N. & Krause, S. J. The Pain Disability Index: psychometric and validity data. Arch Phys Med Rehabil 68, 438-441 (1987).
29. Freynhagen, R., Baron, R., Gockel, U. & Tolle, T. R. painDETECT: a new screening questionnaire to identify neuropathic components in patients with back pain. Curr Med Res Opin 22, 1911-1920 (2006).
30. Melzack, R. The short-form McGill Pain Questionnaire. Pain 30, 191-197 (1987).
31. Sullivan, M. J., Bishop, S. R. & Pivik, J. The pain catastrophizing scale: development and validation. Psychological assessment 7, 524 (1995).
32. McCracken, L. M., Zayfert, C. & Gross, R. T. The Pain Anxiety Symptoms Scale: development and validation of a scale to measure fear of pain. Pain 50, 67-73 (1992).
33. Beck, A. T., Steer, R. A., Ball, R. & Ranieri, W. Comparison of Beck Depression Inventories-IA and -II in psychiatric outpatients. J Pers Assess 67, 588-597 (1996).
34. Watson, D., Clark, L. A. & Tellegen, A. Development and validation of brief measures of positive and negative affect: the PANAS scales. J Pers Soc Psychol 54, 1063-1070 (1988).
35. Loggia, M. L., et al. Default mode network connectivity encodes clinical pain: an arterial spin labeling study. Pain 154, 24-33 (2013).
36. Napadow, V., et al. Intrinsic brain connectivity in fibromyalgia is associated with chronic pain intensity. Arthritis Rheum 62, 2545-2555 (2010).
37. Baliki, M. N., Mansour, A. R., Baria, A. T. & Apkarian, A. V. Functional reorganization of the default mode network across chronic pain conditions. PLoS One 9, e106133 (2014).
38. Qaseem, A., Wilt, T. J., McLean, R. M., Forciea, M. A. & Clinical Guidelines Committee of the American College of, P. Noninvasive Treatments for Acute, Subacute, and Chronic Low Back Pain: A Clinical Practice Guideline From the American College of Physicians. Ann Intern Med 166, 514-530 (2017).
39. Maher, C., Underwood, M. & Buchbinder, R. Non-specific low back pain. Lancet 389, 736-747 (2017).
40. Dance, A. Why the sexes don't feel pain the same way. Nature 567, 448-450 (2019).
41. Wenger, H. C. & Cifu, A. S. Treatment of Low Back Pain. JAMA 318, 743-744 (2017).
42. Enthoven, W. T., Roelofs, P. D., Deyo, R. A., van Tulder, M. W. & Koes, B. W. Non-steroidal anti-inflammatory drugs for chronic low back pain. Cochrane Database Syst Rev 2, CD012087 (2016).
43. Coxib, et al. Vascular and upper gastrointestinal effects of non-steroidal anti-inflammatory drugs: meta-analyses of individual participant data from randomised trials. Lancet 382, 769-779 (2013).
44. Baliki, M. N., et al. Chronic pain and the emotional brain: specific brain activity associated with spontaneous fluctuations of intensity of chronic back pain. J Neurosci 26, 12165-12173 (2006).
45. Huang, L., et al. Reproducibility of structural, resting-state BOLD and DTI data between identical scanners. PLoS One 7, e47684 (2012).
46. Baria, A. T., et al. Linking human brain local activity fluctuations to structural and functional network architectures. Neuroimage 73, 144-155 (2013).
47. Power, J. D., Barnes, K. A., Snyder, A. Z., Schlaggar, B. L. & Petersen, S. E. Spurious but systematic correlations in functional connectivity MRI networks arise from subject motion. Neuroimage 59, 2142-2154 (2012).
48. Smith, S. M., De Stefano, N., Jenkinson, M. & Matthews, P. M. Normalized accurate measurement of longitudinal brain change. J Comput Assist Tomogr 25, 466-475 (2001).
49. Smith, S. M., et al. Tract-based spatial statistics: voxelwise analysis of multi-subject diffusion data. Neuroimage 31, 1487-1505 (2006).
50. Schulz, K. F., Altman, D. G. & Moher, D. CONSORT 2010 Statement: Updated Guidelines for Reporting Parallel Group Randomized Trials RESPONSE. Annals of Internal Medicine 154, 291-292 (2011).
51. Doerks, T., Copley, R. R., Schultz, J., Ponting, C. P. & Bork, P. Systematic identification of novel protein domain families associated with nuclear functions. Genome Res 12, 47-56 (2002).
52. Rubinov, M. & Sporns, O. Complex network measures of brain connectivity: uses and interpretations. Neuroimage 52, 1059-1069 (2010).

### Example 2. Treating Acute Pain in a Female Subject

A subject (e.g., a human female patient) suffering from acute pain (e.g., pain resulting from trauma) can be treated by administering to the subject an analgesic agent (e.g., naproxen) and one or more dopaminergic agents (e.g., a first dopaminergic agent, which may be is a D2 agonist (such as carbidopa) and a second dopaminergic agent, which may be a D1 agonist (such as levodopa). The subject may be administered, for example, 250 mg of naproxen three times per day in combination with 12.5 mg of carbidopa three times per day and/or 50 mg of levodopa three times per day. The treatment may continue for, e.g., 1-24 weeks, such as for a treatment period of about 4 weeks, at which point the subject's pain level may be assessed (e.g., using a pain index described herein). If the subject exhibits a reduced level of pain at the conclusion of the initial treatment period relative to the level of pain experienced by the subject prior to commencement of treatment, the subject may continue to receive treatment at these initial dosage amounts, or treatment may be discontinued if no residual pain is experienced.

However, if the subject does not exhibit a reduced level of pain at the conclusion of the initial treatment period relative to the level of pain experienced by the subject prior to commencement of treatment, the subject may be administered 250 mg of naproxen three times per day in combination with an elevated dose of carbidopa and/or levodopa, such as 25 mg of carbidopa three times per day and/or 100 mg of levodopa three times per day. The subject's pain level may then be re-assessed following a second treatment period of 1-24 weeks (e.g., about 4 weeks). If the subject exhibits a reduced level of pain at the conclusion of the second treatment period relative to the level of pain experienced by the subject prior to commencement of treatment, the subject may continue to receive treatment at these elevated dosage amounts, or treatment may be discontinued if no residual pain is experienced.

If the subject does not exhibit a reduced level of pain at the conclusion of the second treatment period relative to the level of pain experienced by the subject prior to commencement of treatment, the subject may be administered 250 mg of naproxen three times per day in combination with a further increased dose of carbidopa and/or levodopa, such as 50 mg of carbidopa three times per day and/or 200 mg of levodopa three times per day. The subject's pain level may then be re-assessed following a third treatment period of 1-24 weeks (e.g., about 4 weeks). If the subject exhibits a reduced level of pain at the conclusion of the third treatment period relative to the level of pain experienced by the subject prior to commencement of treatment, the subject may continue to receive treatment at these further increased dosage amounts, or treatment may be discontinued if no residual pain is experienced.

### Example 3. Preventing the Transition from Acute to Chronic Pain in a Female Subject

A subject (e.g., a human female patient) suffering from acute pain (e.g., pain resulting from trauma) and at risk of experiencing a transition from acute pain to chronic pain can be treated by administering to the subject an analgesic agent (e.g., naproxen) and one or more dopaminergic agents (e.g., a first dopaminergic agent, which may be is a D2 agonist (such as carbidopa) and a second dopaminergic agent, which may be a D1 agonist (such as levodopa). The subject may be administered, for example, 250 mg of naproxen three times per day in combination with 12.5 mg of carbidopa three times per day and/or 50 mg of levodopa three times per day. The treatment may continue for, e.g., 1-24 weeks, such as for a treatment period of about 4 weeks, at which point the subject's pain level may be assessed (e.g., using a pain index described herein). If the subject exhibits a reduced level of pain at the conclusion of the initial treatment period relative to the level of pain experienced by the subject prior to commencement of treatment, the subject may continue to receive treatment at these initial dosage amounts, or treatment may be discontinued if no residual pain is experienced.

However, if the subject does not exhibit a reduced level of pain at the conclusion of the initial treatment period relative to the level of pain experienced by the subject prior to commencement of treatment, the subject may be administered 250 mg of naproxen three times per day in combination with an elevated dose of carbidopa and/or levodopa, such as 25 mg of carbidopa three times per day and/or 100 mg of levodopa three times per day. The subject's pain level may then be re-assessed following a second treatment period of 1-24 weeks (e.g., about 4 weeks). If the subject exhibits a reduced level of pain at the conclusion of the second treatment period relative to the level of pain experienced by the subject prior to commencement of treatment, the subject may continue to receive treatment at these elevated dosage amounts, or treatment may be discontinued if no residual pain is experienced.

If the subject does not exhibit a reduced level of pain at the conclusion of the second treatment period relative to the level of pain experienced by the subject prior to commencement of treatment, the subject may be administered 250 mg of naproxen three times per day in combination with a further increased dose of carbidopa and/or levodopa, such as 50 mg of carbidopa three times per day and/or 200 mg of levodopa three times per day. The subject's pain level may then be re-assessed following a third treatment period of 1-24 weeks (e.g., about 4 weeks). If the subject exhibits a reduced level of pain at the conclusion of the third treatment period relative to the level of pain experienced by the subject prior to commencement of treatment, the subject may continue to receive treatment at these further increased dosage amounts, or treatment may be discontinued if no residual pain is experienced.

### Example 4. Treating Chronic Pain in a Female Subject

A subject (e.g., a human female patient) suffering from chronic pain can be treated by administering to the subject an analgesic agent (e.g., naproxen) and one or more dopaminergic agents (e.g., a first dopaminergic agent, which may be is a D2 agonist (such as carbidopa) and a second dopaminergic agent, which may be a D1 agonist (such as levodopa). The subject may be administered, for example, 250 mg of naproxen three times per day in combination with 12.5 mg of carbidopa three times per day and/or 50 mg of levodopa three times per day. The treatment may continue for, e.g., 1-24 weeks, such as for a treatment period of about 4 weeks, at which point the subject's pain level may be assessed (e.g., using a pain index described herein). If the subject exhibits a reduced level of pain at the conclusion of the initial treatment period relative to the level of pain experienced by the subject prior to commencement of treatment, the subject may continue to receive treatment at these initial dosage amounts, or treatment may be discontinued if no residual pain is experienced.

However, if the subject does not exhibit a reduced level of pain at the conclusion of the initial treatment period relative to the level of pain experienced by the subject prior to commencement of treatment, the subject may be administered 250 mg of naproxen three times per day in combination with an elevated dose of carbidopa and/or levodopa, such as 25 mg of carbidopa three times per day and/or 100 mg of levodopa three times per day. The subject's pain level may then be re-assessed following a second treatment period of 1-24 weeks (e.g., about 4 weeks). If the subject exhibits a reduced level of pain at the conclusion of the second treatment period relative to the level of pain experienced by the subject prior to commencement of treatment, the subject may continue to receive treatment at these elevated dosage amounts, or treatment may be discontinued if no residual pain is experienced.

If the subject does not exhibit a reduced level of pain at the conclusion of the second treatment period relative to the level of pain experienced by the subject prior to commencement of treatment, the subject may be administered 250 mg of naproxen three times per day in combination with a further increased dose of carbidopa and/or levodopa, such as 50 mg of carbidopa three times per day and/or 200 mg of levodopa three times per day. The subject's pain level may then be re-assessed following a third treatment period of 1-24 weeks (e.g., about 4 weeks). If the subject exhibits a reduced level of pain at the conclusion of the third treatment period relative to the level of pain experienced by the subject prior to commencement of treatment, the subject may continue to receive treatment at these further increased dosage amounts, or treatment may be discontinued if no residual pain is experienced.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claims.

Other embodiments are within the claims.

1. A method of reducing pain in a subject, the method comprising administering an analgesic agent, a first dopaminergic agent, and a second dopaminergic agent to the subject, wherein:
(i) the analgesic agent is administered to the subject in an amount of from about 50 mg to about 500 mg per dose;
(ii) the first dopaminergic agent is administered to the subject in an amount of from about 5 mg to about 75 mg per dose; and
(iii) the second dopaminergic agent is administered to the subject in an amount of from about 25 mg to about 300 mg per dose.

2. A method of reducing pain in a subject, the method comprising administering an analgesic agent, a first dopaminergic agent, and a second dopaminergic agent to the subject, wherein:
(i) the analgesic agent is administered to the subject in an amount of from about 150 mg to about 1,500 mg per day;
(ii) the first dopaminergic agent is administered to the subject in an amount of from about 20 mg to about 200 mg per day; and
(iii) the second dopaminergic agent is administered to the subject in an amount of from about 100 mg to about 1,000 mg per day.

3. A method of reducing pain in a subject, the method comprising administering an analgesic agent, a first dopaminergic agent, and a second dopaminergic agent to the subject, wherein:
(i) the analgesic agent is administered to the subject in an amount of from about 1,000 mg to about 10,000 mg per week;
(ii) the first dopaminergic agent is administered to the subject in an amount of from about 100 mg to about 1,500 mg per week; and
(iii) the second dopaminergic agent is administered to the subject in an amount of from about 500 mg to about 5,000 mg per week.

4. The method of any one of claims 1-3, wherein the analgesic agent is administered to the subject in an amount of from about 100 mg to about 400 mg per dose.

5. The method of claim 4, wherein the analgesic agent is administered to the subject in an amount of from about 200 mg to about 300 mg per dose.

6. The method of claim 5, wherein the analgesic agent is administered to the subject in an amount of from about 225 mg to about 275 mg per dose.

7. The method of claim 6, wherein the analgesic agent is administered to the subject in an amount of about 250 mg per dose.

8. The method of any one of claims 1-7, wherein the analgesic agent is administered to the subject in one or more doses per 12 hours, 24 hours, 36 hours, 48 hours, or week.

9. The method of claim 8, wherein the analgesic agent is administered to the subject in one or more doses per 24 hours.

10. The method of claim 9, wherein the analgesic agent is administered to the subject in from one to ten doses per day.

11. The method of claim 10, wherein the analgesic agent is administered to the subject in from one to six doses per day.

12. The method of claim 11, wherein the analgesic agent is administered to the subject in a single dose per day.

13. The method of claim 11, wherein the analgesic agent is administered to the subject in two doses per day.

14. The method of claim 11, wherein the analgesic agent is administered to the subject in three doses per day.

15. The method of claim 11, wherein the analgesic agent is administered to the subject in four doses per day.

16. The method of any one of claims 1-15, wherein the analgesic agent is administered to the subject in an amount of from about 200 mg to about 1,200 mg per day.

17. The method of claim 16, wherein the analgesic agent is administered to the subject in an amount of from about 500 mg to about 1.00 mg per day.

18. The method of claim 17, wherein the analgesic agent is administered to the subject in an amount of from about 700 mg to about 800 mg per day.

19. The method of claim 18, wherein the analgesic agent is administered to the subject in an amount of about 750 mg per day.

20. The method of any one of claims 1-19, wherein the analgesic agent is administered to the subject in an amount of from about 2,000 mg to about 9,000 mg per week.

21. The method of claim 20, wherein the analgesic agent is administered to the subject in an amount of from about 3,000 mg to about 7,000 mg per week.

22. The method of claim 21, wherein the analgesic agent is administered to the subject in an amount of from about 5,000 mg to about 6,000 mg per week.

23. The method of claim 22, wherein the analgesic agent is administered to the subject in an amount of about 5,250 mg per week.

24. The method of any one of claims 1-23, wherein the analgesic agent is periodically administered to the subject over the course of a treatment period having a duration of from about 1 day to about 1 year.

25. The method of claim 24, wherein the treatment period has a duration of from about 1 week to about 24 weeks.

26. The method of claim 25, wherein the treatment period has a duration of about 4 weeks.

27. The method of claim 25, wherein the treatment period has a duration of about 8 weeks.

28. The method of claim 25, wherein the treatment period has a duration of about 12 weeks.

29. The method of any one of claims 1-28, wherein the first dopaminergic agent is administered to the subject in an amount of from about 7.5 mg to about 25 mg per dose.

30. The method of claim 29, wherein the first dopaminergic agent is administered to the subject in an amount of from about 9 mg to about 20 mg per dose.

31. The method of claim 30, wherein the first dopaminergic agent is administered to the subject in an amount of from about 10 mg to about 15 mg per dose.

32. The method of claim 31, wherein the first dopaminergic agent is administered to the subject in an amount of about 12.5 mg per dose.

33. The method of any one of claims 1-28, wherein the first dopaminergic agent is administered to the subject in an amount of from about 15 mg to about 50 mg per dose.

34. The method of claim 33, wherein the first dopaminergic agent is administered to the subject in an amount of from about 17 mg to about 35 mg per dose.

35. The method of claim 34, wherein the first dopaminergic agent is administered to the subject in an amount of from about 20 mg to about 30 mg per dose.

36. The method of claim 35, wherein the first dopaminergic agent is administered to the subject in an amount of about 25 mg per dose.

37. The method of any one of claims 1-28, wherein the first dopaminergic agent is administered to the subject in an amount of from about 25 mg to about 75 mg per dose.

38. The method of claim 37, wherein the first dopaminergic agent is administered to the subject in an amount of from about 45 mg to about 65 mg per dose.

39. The method of claim 38, wherein the first dopaminergic agent is administered to the subject in an amount of from about 40 mg to about 60 mg per dose.

40. The method of claim 39, wherein the first dopaminergic agent is administered to the subject in an amount of about 50 mg per dose.

41. The method of any one of claims 1-40, wherein the first dopaminergic agent is administered to the subject in one or more doses per 12 hours, 24 hours, 36 hours, 48 hours, or week.

42. The method of claim 41, wherein the first dopaminergic agent is administered to the subject in one or more doses per 24 hours.

43. The method of claim 42, wherein the first dopaminergic agent is administered to the subject in from one to ten doses per day.

44. The method of claim 43, wherein the first dopaminergic agent is administered to the subject in from one to six doses per day.

45. The method of claim 44, wherein the first dopaminergic agent is administered to the subject in a single dose per day.

46. The method of claim 44, wherein the first dopaminergic agent is administered to the subject in two doses per day.

47. The method of claim 44, wherein the first dopaminergic agent is administered to the subject in three doses per day.

48. The method of claim 44, wherein the first dopaminergic agent is administered to the subject in four doses per day.

49. The method of any one of claims 1-48, wherein the first dopaminergic agent is administered to the subject in an amount of from about 25 mg to about 175 mg per day.

50. The method of claim 49, wherein the first dopaminergic agent is administered to the subject in an amount of from about 30 mg to about 50 mg per day.

51. The method of claim 50, wherein the first dopaminergic agent is administered to the subject in an amount of from about 35 mg to about 40 mg per day.

52. The method of claim 51, wherein the first dopaminergic agent is administered to the subject in an amount of about 37.5 mg per day.

53. The method of any one of claims 1-48, wherein the first dopaminergic agent is administered to the subject in an amount of from about 40 mg to about 100 mg per day.

54. The method of claim 53, wherein the first dopaminergic agent is administered to the subject in an amount of from about 50 mg to about 90 mg per day.

55. The method of claim 54, wherein the first dopaminergic agent is administered to the subject in an amount of from about 60 mg to about 80 mg per day.

56. The method of claim 55, wherein the first dopaminergic agent is administered to the subject in an amount of about 75 mg per day.

57. The method of any one of claims 1-48, wherein the first dopaminergic agent is administered to the subject in an amount of from about 75 mg to about 200 mg per day.

58. The method of claim 57, wherein the first dopaminergic agent is administered to the subject in an amount of from about 125 mg to about 175 mg per day.

59. The method of claim 58, wherein the first dopaminergic agent is administered to the subject in an amount of from about 140 mg to about 160 mg per day.

60. The method of claim 59, wherein the first dopaminergic agent is administered to the subject in an amount of about 150 mg per day.

61. The method of any one of claims 1-60, wherein the first dopaminergic agent is administered to the subject in an amount of from about 200 mg to about 500 mg per week.

62. The method of claim 61, wherein the first dopaminergic agent is administered to the subject in an amount of from about 225 mg to about 300 mg per week.

63. The method of claim 62, wherein the first dopaminergic agent is administered to the subject in an amount of from about 230 mg to about 280 mg per week.

64. The method of claim 63, wherein the first dopaminergic agent is administered to the subject in an amount of about 262.5 mg per week.

65. The method of any one of claims 1-60, wherein the first dopaminergic agent is administered to the subject in an amount of from about 300 mg to about 700 mg per week.

66. The method of claim 65, wherein the first dopaminergic agent is administered to the subject in an amount of from about 400 mg to about 600 mg per week.

67. The method of claim 66, wherein the first dopaminergic agent is administered to the subject in an amount of from about 500 mg to about 550 mg per week.

68. The method of claim 67, wherein the first dopaminergic agent is administered to the subject in an amount of about 525 mg per week.

69. The method of any one of claims 1-60, wherein the first dopaminergic agent is administered to the subject in an amount of from about 500 mg to about 1,500 mg per week.

70. The method of claim 69, wherein the first dopaminergic agent is administered to the subject in an amount of from about 750 mg to about 1,300 mg per week.

71. The method of claim 70, wherein the first dopaminergic agent is administered to the subject in an amount of from about 900 mg to about 1,200 mg per week.

72. The method of claim 71, wherein the first dopaminergic agent is administered to the subject in an amount of about 1,050 mg per week.

73. The method of any one of claims 1-72, wherein the first dopaminergic agent is periodically administered to the subject over the course of a treatment period having a duration of from about 1 day to about 1 year.

74. The method of claim 73, wherein the treatment period has a duration of from about 1 week to about 24 weeks.

75. The method of claim 74, wherein the treatment period has a duration of about 4 weeks.

76. The method of claim 74, wherein the treatment period has a duration of about 8 weeks.

77. The method of claim 74, wherein the treatment period has a duration of about 12 weeks.

78. The method of any one of claims 1-77, wherein the second dopaminergic agent is administered to the subject in an amount of from about 35 mg to about 100 mg per dose.

79. The method of claim 78, wherein the second dopaminergic agent is administered to the subject in an amount of from about 40 mg to about 75 mg per dose.

80. The method of claim 79, wherein the second dopaminergic agent is administered to the subject in an amount of from about 45 mg to about 55 mg per dose.

81. The method of claim 80, wherein the second dopaminergic agent is administered to the subject in an amount of about 50 mg per dose.

82. The method of any one of claims 1-77, wherein the second dopaminergic agent is administered to the subject in an amount of from about 50 mg to about 150 mg per dose.

83. The method of claim 82, wherein the second dopaminergic agent is administered to the subject in an amount of from about 75 mg to about 125 mg per dose.

84. The method of claim 83, wherein the second dopaminergic agent is administered to the subject in an amount of from about 85 mg to about 110 mg per dose.

85. The method of claim 84, wherein the second dopaminergic agent is administered to the subject in an amount of about 100 mg per dose.

86. The method of any one of claims 1-77, wherein the second dopaminergic agent is administered to the subject in an amount of from about 150 mg to about 250 mg per dose.

87. The method of claim 86, wherein the second dopaminergic agent is administered to the subject in an amount of from about 175 mg to about 225 mg per dose.

88. The method of claim 87, wherein the second dopaminergic agent is administered to the subject in an amount of from about 190 mg to about 210 mg per dose.

89. The method of claim 88, wherein the second dopaminergic agent is administered to the subject in an amount of about 200 mg per dose.

90. The method of any one of claims 1-89, wherein the second dopaminergic agent is administered to the subject in one or more doses per 12 hours, 24 hours, 36 hours, 48 hours, or week.

91. The method of claim 90, wherein the second dopaminergic agent is administered to the subject in one or more doses per 24 hours.

92. The method of claim 91, wherein the second dopaminergic agent is administered to the subject in from one to ten doses per day.

93. The method of claim 92, wherein the second dopaminergic agent is administered to the subject in from one to six doses per day.

94. The method of claim 93, wherein the second dopaminergic agent is administered to the subject in a single dose per day.

95. The method of claim 93, wherein the second dopaminergic agent is administered to the subject in two doses per day.

96. The method of claim 93, wherein the second dopaminergic agent is administered to the subject in three doses per day.

97. The method of claim 93, wherein the second dopaminergic agent is administered to the subject in four doses per day.

98. The method of any one of claims 1-97, wherein the second dopaminergic agent is administered to the subject in an amount of from about 100 mg to about 400 mg per day.

99. The method of claim 98, wherein the second dopaminergic agent is administered to the subject in an amount of from about 125 mg to about 200 mg per day.

100. The method of claim 99, wherein the second dopaminergic agent is administered to the subject in an amount of from about 145 mg to about 155 mg per day.

101. The method of claim 100, wherein the second dopaminergic agent is administered to the subject in an amount of about 150 mg per day.

102. The method of any one of claims 1-97, wherein the second dopaminergic agent is administered to the subject in an amount of from about 200 mg to about 400 mg per day.

103. The method of claim 102, wherein the second dopaminergic agent is administered to the subject in an amount of from about 250 mg to about 350 mg per day.

104. The method of claim 103, wherein the second dopaminergic agent is administered to the subject in an amount of from about 275 mg to about 325 mg per day.

105. The method of claim 104, wherein the second dopaminergic agent is administered to the subject in an amount of about 300 mg per day.

106. The method of any one of claims 1-97, wherein the second dopaminergic agent is administered to the subject in an amount of from about 500 mg to about 700 mg per day.

107. The method of claim 106, wherein the second dopaminergic agent is administered to the subject in an amount of from about 550 mg to about 650 mg per day.

108. The method of claim 107, wherein the second dopaminergic agent is administered to the subject in an amount of from about 575 mg to about 625 mg per day.

109. The method of claim 108, wherein the second dopaminergic agent is administered to the subject in an amount of about 600 mg per day.

110. The method of any one of claims 1-109, wherein the second dopaminergic agent is administered to the subject in an amount of from about 750 mg to about 1,300 mg per week.

111. The method of claim 110, wherein the second dopaminergic agent is administered to the subject in an amount of from about 800 mg to about 1,200 mg per week.

112. The method of claim 111, wherein the second dopaminergic agent is administered to the subject in an amount of from about 900 mg to about 1,100 mg per week.

113. The method of claim 112, wherein the second dopaminergic agent is administered to the subject in an amount of about 1,050 mg per week.

114. The method of any one of claims 1-109, wherein the second dopaminergic agent is administered to the subject in an amount of from about 1,500 mg to about 2,500 mg per week.

115. The method of claim 114, wherein the second dopaminergic agent is administered to the subject in an amount of from about 1,700 mg to about 2,300 mg per week.

116. The method of claim 115, wherein the second dopaminergic agent is administered to the subject in an amount of from about 1,900 mg to about 2,200 mg per week.

117. The method of claim 116, wherein the second dopaminergic agent is administered to the subject in an amount of about 2,100 mg per week.

118. The method of any one of claims 1-109, wherein the second dopaminergic agent is administered to the subject in an amount of from about 3,500 mg to about 4,500 mg per week.

119. The method of claim 118, wherein the second dopaminergic agent is administered to the subject in an amount of from about 3,700 mg to about 4,400 mg per week.

120. The method of claim 119, wherein the second dopaminergic agent is administered to the subject in an amount of from about 4,100 mg to about 4,300 mg per week.

121. The method of claim 120, wherein the second dopaminergic agent is administered to the subject in an amount of about 4,200 mg per week.

122. The method of any one of claims 1-121, wherein the second dopaminergic agent is periodically administered to the subject over the course of a treatment period having a duration of from about 1 day to about 1 year.

123. The method of claim 122, wherein the treatment period has a duration of from about 1 week to about 24 weeks.

124. The method of claim 123, wherein the treatment period has a duration of about 4 weeks.

125. The method of claim 123, wherein the treatment period has a duration of about 8 weeks.

126. The method of claim 123, wherein the treatment period has a duration of about 12 weeks.

127. The method of any one of claims 1-126, wherein the first dopaminergic agent is administered to the subject:
(i) in an amount of from about 7.5 mg to about 25 mg per dose during a first treatment period;
(ii) in an amount of from about 15 mg to about 50 mg per dose during a second treatment period; and
(iii) in an amount of from about 25 mg to about 75 mg per dose during a third treatment period,
optionally wherein the first dopaminergic agent is administered to the subject in three doses per day during each treatment period.

128. The method of claim 127, wherein the first dopaminergic agent is administered to the subject:
(i) in an amount of from about 9 mg to about 20 mg per dose during a first treatment period;
(ii) in an amount of from about 17 mg to about 35 mg per dose during a second treatment period; and
(iii) in an amount of from about 45 mg to about 65 mg per dose during a third treatment period,
optionally wherein the first dopaminergic agent is administered to the subject in three doses per day during each treatment period.

129. The method of claim 128, wherein the first dopaminergic agent is administered to the subject:
(i) in an amount of from about 10 mg to about 15 mg per dose during a first treatment period;
(ii) in an amount of from about 20 mg to about 30 mg per dose during a second treatment period; and
(iii) in an amount of from about 40 mg to about 60 mg per dose during a third treatment period,
optionally wherein the first dopaminergic agent is administered to the subject in three doses per day during each treatment period.

130. The method of claim 129, wherein the first dopaminergic agent is administered to the subject:
(i) in an amount of about 12.5 mg per dose during a first treatment period;
(ii) in an amount of about 25 mg per dose during a second treatment period; and
(iii) in an amount of about 50 mg per dose during a third treatment period,
optionally wherein the first dopaminergic agent is administered to the subject in three doses per day during each treatment period.

131. The method of any one of claims 127-130, wherein the first, second, and/or third treatment period has a duration of from about 1 week to about 24 weeks.

132. The method of claim 131, wherein the first, second, and/or third treatment period has a duration of about 4 weeks.

133. The method of any one of claims 1-132, wherein the second dopaminergic agent is administered to the subject:
(i) in an amount of from about 35 mg to about 100 mg per dose during a first treatment period;
(ii) in an amount of from about 50 mg to about 150 mg per dose during a second treatment period; and
(iii) in an amount of from about 150 mg to about 250 mg per dose during a third treatment period,
optionally wherein the second dopaminergic agent is administered to the subject in three doses per day during each treatment period.

134. The method of claim 133, wherein the second dopaminergic agent is administered to the subject:
(i) in an amount of from about 40 mg to about 75 mg per dose during a first treatment period;
(ii) in an amount of from about 75 mg to about 125 mg per dose during a second treatment period; and
(iii) in an amount of from about 175 mg to about 225 mg per dose during a third treatment period,
optionally wherein the second dopaminergic agent is administered to the subject in three doses per day during each treatment period.

135. The method of claim 134, wherein the second dopaminergic agent is administered to the subject:
(i) in an amount of from about 45 mg to about 55 mg per dose during a first treatment period;
(ii) in an amount of from about 85 mg to about 110 mg per dose during a second treatment period; and
(iii) in an amount of from about 190 mg to about 210 mg per dose during a third treatment period,
optionally wherein the second dopaminergic agent is administered to the subject in three doses per day during each treatment period.

136. The method of claim 135, wherein the second dopaminergic agent is administered to the subject:
(i) in an amount of about 50 mg per dose during a first treatment period;
(ii) in an amount of about 100 mg per dose during a second treatment period; and
(iii) in an amount of about 200 mg per dose during a third treatment period,
optionally wherein the second dopaminergic agent is administered to the subject in three doses per day during each treatment period.

137. The method of any one of claims 133-136, wherein the first, second, and/or third treatment period has a duration of from about 1 week to about 24 weeks.

138. The method of claim 137, wherein the first, second, and/or third treatment period has a duration of about 4 weeks.

139. The method of any one of claims 1-138, wherein the analgesic agent, first dopaminergic agent, and second dopaminergic agent are administered to the subject in a single pharmaceutical composition.

140. The method of any one of claims 1-138, wherein the analgesic agent is administered to the subject in a first pharmaceutical composition and the first dopaminergic agent and second dopaminergic agent are administered to the subject in a separate pharmaceutical composition.

141. The method of any one of claims 1-140, wherein the analgesic agent is a non-steroidal anti-inflammatory drug (NSAID).

142. The method of claim 141, wherein the analgesic agent is naproxen, aceclofenac, acemetacin, acetaminophen, aloxiprin, aspirin, benorilate, bromfenac, celecoxib, deracoxib, diclofenac, diflunisal, ethenzamide, etodolac, etofenamate, etoricoxib, fenbufen, fenoprofen, flufenamic acid, flurbiprofen, lonazolac, lornoxicam, ibuprofen, indomethacin, isoxicam, kebuzone, ketoprofen, ketorolac, licofelone, loxoprofen, lumiracoxib, meclofenamic acid, mefenamic acid, meloxicam, metamizol, mofebutazone, naproxen, nabumetone, niflumic acid, nimesulide, oxaprozin, oxyphenbutazone, parecoxib, phenidone, phenylbutazone, piroxicam, propacetamol, propyphenazone, rofecoxib, salicylamide, sulfinpyrazone, sulindac, suprofen, tiaprofenic acid, tenoxicam, tolmetin, or valdecoxib.

143. The method of claim 142, wherein the analgesic agent is naproxen.

144. The method of any one of claims 1-140, wherein the analgesic agent is an anticonvulsant.

145. The method of claim 144, wherein the analgesic agent is pregabalin, lamotrigine, topiramate, oxcarbazepine, tiagabine, levetiracetam, zonisamide, phenytoin, carbamazepine, gabapentin, or ethosuximide.

146. The method of any one of claims 1-140, wherein the analgesic agent is paracetamol.

147. The method of any one of claims 1-146, wherein the first dopaminergic agent and/or second dopaminergic agent is a dopamine receptor agonist or a precursor thereof.

148. The method of any one of claims 1-147, wherein the first dopaminergic agent is a D2 agonist.

149. The method of claim 148, wherein the first dopaminergic agent is pramipexole or carbidopa.

150. The method of claim 149, wherein the first dopaminergic agent is carbidopa.

151. The method of any one of claims 1-150, wherein the second dopaminergic agent is a D1 agonist.

152. The method of claim 151, wherein the second dopaminergic agent is levodopa.

153. The method of any one of claims 1-152, the method comprising administering an additional therapeutic agent to the subject.

154. The method of claim 153, wherein the additional therapeutic agent is a chemotherapeutic agent, an anti-inflammatory agent, an antidepressant, an antiemetic agent, or a muscle relaxant.

155. The method of any one of claims 1-154, wherein the analgesic agent, first dopaminergic agent, and/or second dopaminergic agent is administered to the patient orally, intravenously, intramuscularly, intravitreally, ocularly, intraocularly, itraorbitally, intradermally, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, topically, by inhalation, by injection, by implantation, by infusion, by continuous infusion, by localized perfusion bathing target cells directly, by catheter, by lavage, in creams, or in lipid compositions.

156. The method of claim 155, wherein the analgesic agent, first dopaminergic agent, and/or second dopaminergic agent is administered to the patient orally.

157. The method of any one of claims 1-156, wherein the pain is acute pain.

158. The method of claim 157, wherein the acute pain is caused by a sports-related injury, a military injury, other physical trauma, a surgical procedure, cancer, infection, inflammation, or childbirth.

159. The method of any one of claims 1-158, wherein the subject is at risk of transitioning from experiencing acute pain to experiencing chronic pain.

160. The method of any one of claims 1-159, wherein the method prevents transition of acute pain to chronic pain.

161. The method of any one of claims 1-156, wherein the pain is chronic pain.

162. The method of any one of claims 159-161, wherein the chronic pain is peripheral neuropathic pain, post-herpetic neuralgia, diabetic neuropathic pain, neuropathic cancer pain, failed back-surgery syndrome, trigeminal neuralgia, phantom limb pain, central neuropathic pain, multiple sclerosis related pain, Parkinson disease-related pain, post-stroke pain, post-traumatic spinal cord injury pain, pain from dementia, musculoskeletal pain, osteoarthritic pain, fibromyalgia syndrome, inflammatory pain, rheumatoid arthritis, endometriosis, migraine, cluster headache, tension headache syndrome, facial pain, headache caused by other diseases, visceral pain, interstitial cystitis, irritable bowel syndrome, chronic pelvic pain syndrome, lower back pain, neck and shoulder pain, burning mouth syndrome, or complex regional pain syndrome.

163. The method of any one of claims 1-162, wherein administration of the analgesic agent, first dopaminergic agent, and second dopaminergic agent commences prior to the onset of the pain.

164. The method of claim 163, wherein administration of the analgesic agent, first dopaminergic agent, and second dopaminergic agent commences no earlier than two months prior to the onset of the pain.

165. The method of any one of claims 1-162, wherein administration of the analgesic agent, first dopaminergic agent, and second dopaminergic agent commences after the onset of the pain.

166. The method of claim 165, wherein administration of the analgesic agent, first dopaminergic agent, and second dopaminergic agent commences no later than 3 months after the onset of the pain.

167. The method of any one of claims 1-166, wherein the subject is a mammal.

168. The method of claim 167, wherein the subject is a human.

169. The method of any one of claims 1-168, wherein the subject is a female.

170. The method of any one of claims 1-169, wherein the subject is a male.

171. The method of any one of claims 1-170, wherein the method further comprises performing a brain scan on the subject, optionally wherein the brain scan reveals that the subject has reduced connectivity between the subject's nucleus accumbens (NAc) and the subject's medial prefrontal cortex (mPFC) relative to a reference level of NAc-mPFC connectivity.

172. The method of any one of claims 1-170, wherein the subject has been identified as having a reduced connectivity between the subject's nucleus accumbens (NAc) and the subject's medial prefrontal cortex (mPFC) relative to a reference level of NAc-mPFC connectivity.

173. The method of claim 171 or 172, wherein the brain scan is a functional magnetic resonance imaging (fMRI) technique.

174. The method of any one of claims 171-173, wherein the reference level is the median level of NAc-mPFC connectivity in a population of patients being tested for likelihood of responding to administration of the analgesic agent, first dopaminergic agent, and/or second dopaminergic agent.

175. The method of any one of claims 171-174, wherein the reference level is a z-transformed correlation score of NAc-mPFC connectivity, optionally wherein the subject's NAc-mPFC connectivity is compared to the reference level by assessing the subject's NAc-mPFC z-transformed correlation score.

176. The method of claim 175, wherein the reference level is from about 0.1 to about 0.4.

177. The method of claim 176, wherein the reference level is from about 0.15 to about 0.3.

178. The method of claim 177, wherein the reference level is about 0.2.

179. A pharmaceutical composition comprising an analgesic agent in an amount of from about 50 mg to about 500 mg, a first dopaminergic agent in an amount of from about 5 mg to about 75 mg, and a second dopaminergic agent an amount of from about 25 mg to about 300 mg.

180. The pharmaceutical composition of claim 179, comprising the analgesic agent in an amount of from about 100 mg to about 400 mg.

181. The pharmaceutical composition of claim 180, comprising the analgesic agent in an amount of from about 200 mg to about 300 mg.

182. The pharmaceutical composition of claim 181, comprising the analgesic agent in an amount of from about 225 mg to about 275 mg.

183. The pharmaceutical composition of claim 182, comprising the analgesic agent in an amount of about 250 mg.

184. The pharmaceutical composition of any one of claims 179-183, comprising the first dopaminergic agent in an amount of from about 7.5 mg to about 25 mg.

185. The pharmaceutical composition of claim 184, comprising the first dopaminergic in an amount of from about 9 mg to about 20 mg.

186. The pharmaceutical composition of claim 185, comprising the first dopaminergic in an amount of from about 10 mg to about 15 mg.

187. The pharmaceutical composition of claim 186, comprising the first dopaminergic in an amount of about 12.5 mg.

188. The pharmaceutical composition of any one of claims 179-183, comprising the first dopaminergic agent in an amount of from about 15 mg to about 50 mg.

189. The pharmaceutical composition of claim 188, comprising the first dopaminergic in an amount of from about 17 mg to about 35 mg.

190. The pharmaceutical composition of claim 189, comprising the first dopaminergic in an amount of from about 20 mg to about 30 mg.

191. The pharmaceutical composition of claim 190, comprising the first dopaminergic in an amount of about 25 mg.

192. The pharmaceutical composition of any one of claims 179-183, comprising the first dopaminergic agent in an amount of from about 25 mg to about 75 mg.

193. The pharmaceutical composition of claim 192, comprising the first dopaminergic in an amount of from about 45 mg to about 65 mg.

194. The pharmaceutical composition of claim 193, comprising the first dopaminergic in an amount of from about 40 mg to about 60 mg.

195. The pharmaceutical composition of claim 194, comprising the first dopaminergic in an amount of about 50 mg.

196. The pharmaceutical composition of any one of claims 179-195, comprising the second dopaminergic agent in an amount of from about 35 mg to about 100 mg.

197. The pharmaceutical composition of claim 196, comprising the second dopaminergic agent in an amount of from about 40 mg to about 75 mg.

198. The pharmaceutical composition of claim 197, comprising the second dopaminergic agent in an amount of from about 45 mg to about 55 mg.

199. The pharmaceutical composition of claim 198, comprising the second dopaminergic agent in an amount of from about 50 mg.

200. The pharmaceutical composition of any one of claims 179-195, comprising the second dopaminergic agent in an amount of from about 50 mg to about 150 mg.

201. The pharmaceutical composition of claim 200, comprising the second dopaminergic agent in an amount of from about 75 mg to about 125 mg.

202. The pharmaceutical composition of claim 201, comprising the second dopaminergic agent in an amount of from about 85 mg to about 110 mg.

203. The pharmaceutical composition of claim 202, comprising the second dopaminergic agent in an amount of from about 100 mg.

204. The pharmaceutical composition of any one of claims 179-195, comprising the second dopaminergic agent in an amount of from 150 mg to about 250 mg.

205. The pharmaceutical composition of claim 204, comprising the second dopaminergic agent in an amount of from about 175 mg to about 225 mg.

206. The pharmaceutical composition of claim 205, comprising the second dopaminergic agent in an amount of from about 190 mg to about 210 mg.

207. The pharmaceutical composition of claim 206, comprising the second dopaminergic agent in an amount of from about 200 mg.

208. The pharmaceutical composition of any one of claims 179-207, wherein the analgesic agent is a NSAID.

209. The pharmaceutical composition of claim 208, wherein the analgesic agent is naproxen, aceclofenac, acemetacin, acetaminophen, aloxiprin, aspirin, benorilate, bromfenac, celecoxib, deracoxib, diclofenac, diflunisal, ethenzamide, etodolac, etofenamate, etoricoxib, fenbufen, fenoprofen, flufenamic acid, flurbiprofen, lonazolac, lornoxicam, ibuprofen, indomethacin, isoxicam, kebuzone, ketoprofen, ketorolac, licofelone, loxoprofen, lumiracoxib, meclofenamic acid, mefenamic acid, meloxicam, metamizol, mofebutazone, naproxen, nabumetone, niflumic acid, nimesulide, oxaprozin, oxyphenbutazone, parecoxib, phenidone, phenylbutazone, piroxicam, propacetamol, propyphenazone, rofecoxib, salicylamide, sulfinpyrazone, sulindac, suprofen, tiaprofenic acid, tenoxicam, tolmetin, or valdecoxib.

210. The pharmaceutical composition of claim 209, wherein the analgesic agent is naproxen.

211. The pharmaceutical composition of any one of claims 179-207, wherein the analgesic agent is an anticonvulsant.

212. The pharmaceutical composition of claim 211, wherein the analgesic agent is pregabalin, lamotrigine, topiramate, oxcarbazepine, tiagabine, levetiracetam, zonisamide, phenytoin, carbamazepine, gabapentin, or ethosuximide.

213. The pharmaceutical composition of any one of claims 179-207, wherein the analgesic agent is paracetamol.

214. The pharmaceutical composition of any one of claims 179-213, wherein the first dopaminergic agent and/or second dopaminergic agent is a dopamine receptor agonist or a precursor thereof.

215. The pharmaceutical composition of any one of claims 179-214, wherein the first dopaminergic agent is a D2 agonist.

216. The pharmaceutical composition of claim 215, wherein the first dopaminergic agent is pramipexole or carbidopa.

217. The pharmaceutical composition of claim 216, wherein the first dopaminergic agent is carbidopa.

218. The pharmaceutical composition of any one of claims 179-217, wherein the second dopaminergic agent is a D1 agonist.

219. The pharmaceutical composition of claim 218 wherein the second dopaminergic agent is levodopa.

220. The pharmaceutical composition of any one of claims 179-219, wherein the composition is a tablet, capsule, gel cap, powder, liquid solution, or liquid suspension.

221. The pharmaceutical composition of any one of claims 179-220, wherein the composition is formulated for sustained release.

222. The pharmaceutical composition of any one of claims 179-219, wherein the composition is an implanted device.

223. A kit comprising the pharmaceutical composition of any one of claims 179-222 and a package insert instructing a user of the kit to administer the pharmaceutical composition to a subject at risk of acute or chronic pain, having acute or chronic pain, or at risk of transitioning from acute pain to chronic pain.

224. A kit comprising an analgesic agent, a first dopaminergic agent, a second dopaminergic agent, and a package insert instructing a user of the kit to administer the analgesic agent, first dopaminergic agent, and second dopaminergic agent to a subject in accordance with the method of any one of claims 1-178.

225. The method of claim 1, wherein the analgesic agent is administered to the subject in one or more doses per 12 hours, 24 hours, 36 hours, 48 hours, or week.

226. The method of claim 225, wherein the analgesic agent is administered to the subject in one or more doses per 24 hours.

227. The method of claim 226, wherein the analgesic agent is administered to the subject in from one to ten doses per day.

228. The method of claim 227, wherein the analgesic agent is administered to the subject in from one to six doses per day.

229. The method of claim 228, wherein the analgesic agent is administered to the subject in a single dose per day.

230. The method of claim 228, wherein the analgesic agent is administered to the subject in two doses per day.

231. The method of claim 228, wherein the analgesic agent is administered to the subject in three doses per day.

232. The method of claim 228, wherein the analgesic agent is administered to the subject in four doses per day.

233. The method of claim 1, wherein the analgesic agent is administered to the subject in an amount of from about 200 mg to about 1,200 mg per day.

234. The method of claim 233, wherein the analgesic agent is administered to the subject in an amount of from about 500 mg to about 1.00 mg per day.

235. The method of claim 234, wherein the analgesic agent is administered to the subject in an amount of from about 700 mg to about 800 mg per day.

236. The method of claim 235, wherein the analgesic agent is administered to the subject in an amount of about 750 mg per day.

237. The method of claim 1, wherein the analgesic agent is administered to the subject in an amount of from about 2,000 mg to about 9,000 mg per week.

238. The method of claim 237, wherein the analgesic agent is administered to the subject in an amount of from about 3,000 mg to about 7,000 mg per week.

239. The method of claim 238, wherein the analgesic agent is administered to the subject in an amount of from about 5,000 mg to about 6,000 mg per week.

240. The method of claim 239, wherein the analgesic agent is administered to the subject in an amount of about 5,250 mg per week.

241. The method of claim 1, wherein the analgesic agent is periodically administered to the subject over the course of a treatment period having a duration of from about 1 day to about 1 year.

242. The method of claim 241, wherein the treatment period has a duration of from about 1 week to about 24 weeks.

243. The method of claim 242, wherein the treatment period has a duration of about 4 weeks.

244. The method of claim 242, wherein the treatment period has a duration of about 8 weeks.

245. The method of claim 242, wherein the treatment period has a duration of about 12 weeks.

246. The method of claim 1, wherein the first dopaminergic agent is administered to the subject in an amount of from about 7.5 mg to about 25 mg per dose.

247. The method of claim 246, wherein the first dopaminergic agent is administered to the subject in an amount of from about 9 mg to about 20 mg per dose.

248. The method of claim 247, wherein the first dopaminergic agent is administered to the subject in an amount of from about 10 mg to about 15 mg per dose.

249. The method of claim 248, wherein the first dopaminergic agent is administered to the subject in an amount of about 12.5 mg per dose.

250. The method of claim 1, wherein the first dopaminergic agent is administered to the subject in an amount of from about 15 mg to about 50 mg per dose.

251. The method of claim 250, wherein the first dopaminergic agent is administered to the subject in an amount of from about 17 mg to about 35 mg per dose.

252. The method of claim 251, wherein the first dopaminergic agent is administered to the subject in an amount of from about 20 mg to about 30 mg per dose.

253. The method of claim 252, wherein the first dopaminergic agent is administered to the subject in an amount of about 25 mg per dose.

254. The method of claim 1, wherein the first dopaminergic agent is administered to the subject in an amount of from about 25 mg to about 75 mg per dose.

255. The method of claim 254, wherein the first dopaminergic agent is administered to the subject in an amount of from about 45 mg to about 65 mg per dose.

256. The method of claim 255, wherein the first dopaminergic agent is administered to the subject in an amount of from about 40 mg to about 60 mg per dose.

257. The method of claim 256, wherein the first dopaminergic agent is administered to the subject in an amount of about 50 mg per dose.

258. The method of claim 1, wherein the first dopaminergic agent is administered to the subject in one or more doses per 12 hours, 24 hours, 36 hours, 48 hours, or week.

259. The method of claim 258, wherein the first dopaminergic agent is administered to the subject in one or more doses per 24 hours.

260. The method of claim 259, wherein the first dopaminergic agent is administered to the subject in from one to ten doses per day.

261. The method of claim 260, wherein the first dopaminergic agent is administered to the subject in from one to six doses per day.

262. The method of claim 261, wherein the first dopaminergic agent is administered to the subject in a single dose per day.

263. The method of claim 261, wherein the first dopaminergic agent is administered to the subject in two doses per day.

264. The method of claim 261, wherein the first dopaminergic agent is administered to the subject in three doses per day.

265. The method of claim 261, wherein the first dopaminergic agent is administered to the subject in four doses per day.

266. The method of claim 1, wherein the first dopaminergic agent is administered to the subject in an amount of from about 25 mg to about 175 mg per day.

267. The method of claim 266, wherein the first dopaminergic agent is administered to the subject in an amount of from about 30 mg to about 50 mg per day.

268. The method of claim 267, wherein the first dopaminergic agent is administered to the subject in an amount of from about 35 mg to about 40 mg per day.

269. The method of claim 268, wherein the first dopaminergic agent is administered to the subject in an amount of about 37.5 mg per day.

270. The method of claim 1, wherein the first dopaminergic agent is administered to the subject in an amount of from about 40 mg to about 100 mg per day.

271. The method of claim 270, wherein the first dopaminergic agent is administered to the subject in an amount of from about 50 mg to about 90 mg per day.

272. The method of claim 271, wherein the first dopaminergic agent is administered to the subject in an amount of from about 60 mg to about 80 mg per day.

273. The method of claim 272, wherein the first dopaminergic agent is administered to the subject in an amount of about 75 mg per day.

274. The method of claim 1, wherein the first dopaminergic agent is administered to the subject in an amount of from about 75 mg to about 200 mg per day.

275. The method of claim 274, wherein the first dopaminergic agent is administered to the subject in an amount of from about 125 mg to about 175 mg per day.

276. The method of claim 275, wherein the first dopaminergic agent is administered to the subject in an amount of from about 140 mg to about 160 mg per day.

277. The method of claim 276, wherein the first dopaminergic agent is administered to the subject in an amount of about 150 mg per day.

278. The method of claim 1, wherein the first dopaminergic agent is administered to the subject in an amount of from about 200 mg to about 500 mg per week.

279. The method of claim 278, wherein the first dopaminergic agent is administered to the subject in an amount of from about 225 mg to about 300 mg per week.

280. The method of claim 279, wherein the first dopaminergic agent is administered to the subject in an amount of from about 230 mg to about 280 mg per week.

281. The method of claim 280, wherein the first dopaminergic agent is administered to the subject in an amount of about 262.5 mg per week.

282. The method of claim 1, wherein the first dopaminergic agent is administered to the subject in an amount of from about 300 mg to about 700 mg per week.

283. The method of claim 282, wherein the first dopaminergic agent is administered to the subject in an amount of from about 400 mg to about 600 mg per week.

284. The method of claim 283, wherein the first dopaminergic agent is administered to the subject in an amount of from about 500 mg to about 550 mg per week.

285. The method of claim 284, wherein the first dopaminergic agent is administered to the subject in an amount of about 525 mg per week.

286. The method of claim 1, wherein the first dopaminergic agent is administered to the subject in an amount of from about 500 mg to about 1,500 mg per week.

287. The method of claim 286, wherein the first dopaminergic agent is administered to the subject in an amount of from about 750 mg to about 1,300 mg per week.

288. The method of claim 287, wherein the first dopaminergic agent is administered to the subject in an amount of from about 900 mg to about 1,200 mg per week.

289. The method of claim 288, wherein the first dopaminergic agent is administered to the subject in an amount of about 1,050 mg per week.

290. The method of claim 1, wherein the first dopaminergic agent is periodically administered to the subject over the course of a treatment period having a duration of from about 1 day to about 1 year.

291. The method of claim 290, wherein the treatment period has a duration of from about 1 week to about 24 weeks.

292. The method of claim 291, wherein the treatment period has a duration of about 4 weeks.

293. The method of claim 291, wherein the treatment period has a duration of about 8 weeks.

294. The method of claim 291, wherein the treatment period has a duration of about 12 weeks.

295. The method of claim 1, wherein the second dopaminergic agent is administered to the subject in an amount of from about 35 mg to about 100 mg per dose.

296. The method of claim 295, wherein the second dopaminergic agent is administered to the subject in an amount of from about 40 mg to about 75 mg per dose.

297. The method of claim 296, wherein the second dopaminergic agent is administered to the subject in an amount of from about 45 mg to about 55 mg per dose.

298. The method of claim 297, wherein the second dopaminergic agent is administered to the subject in an amount of about 50 mg per dose.

299. The method of claim 1, wherein the second dopaminergic agent is administered to the subject in an amount of from about 50 mg to about 150 mg per dose.

300. The method of claim 299, wherein the second dopaminergic agent is administered to the subject in an amount of from about 75 mg to about 125 mg per dose.

301. The method of claim 300, wherein the second dopaminergic agent is administered to the subject in an amount of from about 85 mg to about 110 mg per dose.

302. The method of claim 301, wherein the second dopaminergic agent is administered to the subject in an amount of about 100 mg per dose.

303. The method of claim 1, wherein the second dopaminergic agent is administered to the subject in an amount of from about 150 mg to about 250 mg per dose.

304. The method of claim 303, wherein the second dopaminergic agent is administered to the subject in an amount of from about 175 mg to about 225 mg per dose.

305. The method of claim 304, wherein the second dopaminergic agent is administered to the subject in an amount of from about 190 mg to about 210 mg per dose.

306. The method of claim 305, wherein the second dopaminergic agent is administered to the subject in an amount of about 200 mg per dose.

307. The method of claim 1, wherein the second dopaminergic agent is administered to the subject in one or more doses per 12 hours, 24 hours, 36 hours, 48 hours, or week.

308. The method of claim 307, wherein the second dopaminergic agent is administered to the subject in one or more doses per 24 hours.

309. The method of claim 308, wherein the second dopaminergic agent is administered to the subject in from one to ten doses per day.

310. The method of claim 309, wherein the second dopaminergic agent is administered to the subject in from one to six doses per day.

311. The method of claim 310, wherein the second dopaminergic agent is administered to the subject in a single dose per day.

312. The method of claim 310, wherein the second dopaminergic agent is administered to the subject in two doses per day.

313. The method of claim 310, wherein the second dopaminergic agent is administered to the subject in three doses per day.

314. The method of claim 310, wherein the second dopaminergic agent is administered to the subject in four doses per day.

315. The method of claim 1, wherein the second dopaminergic agent is administered to the subject in an amount of from about 100 mg to about 400 mg per day.

316. The method of claim 315, wherein the second dopaminergic agent is administered to the subject in an amount of from about 125 mg to about 200 mg per day.

317. The method of claim 316, wherein the second dopaminergic agent is administered to the subject in an amount of from about 145 mg to about 155 mg per day.

318. The method of claim 317, wherein the second dopaminergic agent is administered to the subject in an amount of about 150 mg per day.

319. The method of claim 1, wherein the second dopaminergic agent is administered to the subject in an amount of from about 200 mg to about 400 mg per day.

320. The method of claim 319, wherein the second dopaminergic agent is administered to the subject in an amount of from about 250 mg to about 350 mg per day.

321. The method of claim 320, wherein the second dopaminergic agent is administered to the subject in an amount of from about 275 mg to about 325 mg per day.

322. The method of claim 321, wherein the second dopaminergic agent is administered to the subject in an amount of about 300 mg per day.

323. The method of claim 1, wherein the second dopaminergic agent is administered to the subject in an amount of from about 500 mg to about 700 mg per day.

324. The method of claim 323, wherein the second dopaminergic agent is administered to the subject in an amount of from about 550 mg to about 650 mg per day.

325. The method of claim 324, wherein the second dopaminergic agent is administered to the subject in an amount of from about 575 mg to about 625 mg per day.

326. The method of claim 325, wherein the second dopaminergic agent is administered to the subject in an amount of about 600 mg per day.

327. The method of claim 1, wherein the second dopaminergic agent is administered to the subject in an amount of from about 750 mg to about 1,300 mg per week.

328. The method of claim 327, wherein the second dopaminergic agent is administered to the subject in an amount of from about 800 mg to about 1,200 mg per week.

329. The method of claim 328, wherein the second dopaminergic agent is administered to the subject in an amount of from about 900 mg to about 1,100 mg per week.

330. The method of claim 329, wherein the second dopaminergic agent is administered to the subject in an amount of about 1,050 mg per week.

331. The method of claim 1, wherein the second dopaminergic agent is administered to the subject in an amount of from about 1,500 mg to about 2,500 mg per week.

332. The method of claim 331, wherein the second dopaminergic agent is administered to the subject in an amount of from about 1,700 mg to about 2,300 mg per week.

333. The method of claim 332, wherein the second dopaminergic agent is administered to the subject in an amount of from about 1,900 mg to about 2,200 mg per week.

334. The method of claim 333, wherein the second dopaminergic agent is administered to the subject in an amount of about 2,100 mg per week.

335. The method of claim 1, wherein the second dopaminergic agent is administered to the subject in an amount of from about 3,500 mg to about 4,500 mg per week.

336. The method of claim 335, wherein the second dopaminergic agent is administered to the subject in an amount of from about 3,700 mg to about 4,400 mg per week.

337. The method of claim 336, wherein the second dopaminergic agent is administered to the subject in an amount of from about 4,100 mg to about 4,300 mg per week.

338. The method of claim 337, wherein the second dopaminergic agent is administered to the subject in an amount of about 4,200 mg per week.

339. The method of claim 1, wherein the second dopaminergic agent is periodically administered to the subject over the course of a treatment period having a duration of from about 1 day to about 1 year.

340. The method of claim 339, wherein the treatment period has a duration of from about 1 week to about 24 weeks.

341. The method of claim 340, wherein the treatment period has a duration of about 4 weeks.

342. The method of claim 340, wherein the treatment period has a duration of about 8 weeks.

343. The method of claim 340, wherein the treatment period has a duration of about 12 weeks.

344. The method of claim 1, wherein the first dopaminergic agent is administered to the subject:
(i) in an amount of from about 7.5 mg to about 25 mg per dose during a first treatment period;
(ii) in an amount of from about 15 mg to about 50 mg per dose during a second treatment period; and
(iii) in an amount of from about 25 mg to about 75 mg per dose during a third treatment period,
optionally wherein the first dopaminergic agent is administered to the subject in three doses per day during each treatment period.

345. The method of claim 344, wherein the first dopaminergic agent is administered to the subject:
(i) in an amount of from about 9 mg to about 20 mg per dose during a first treatment period;
(ii) in an amount of from about 17 mg to about 35 mg per dose during a second treatment period; and
(iii) in an amount of from about 45 mg to about 65 mg per dose during a third treatment period,
optionally wherein the first dopaminergic agent is administered to the subject in three doses per day during each treatment period.

346. The method of claim 345, wherein the first dopaminergic agent is administered to the subject:
(i) in an amount of from about 10 mg to about 15 mg per dose during a first treatment period;
(ii) in an amount of from about 20 mg to about 30 mg per dose during a second treatment period; and
(iii) in an amount of from about 40 mg to about 60 mg per dose during a third treatment period,
optionally wherein the first dopaminergic agent is administered to the subject in three doses per day during each treatment period.

347. The method of claim 346, wherein the first dopaminergic agent is administered to the subject:
(i) in an amount of about 12.5 mg per dose during a first treatment period;
(ii) in an amount of about 25 mg per dose during a second treatment period; and
(iii) in an amount of about 50 mg per dose during a third treatment period,
optionally wherein the first dopaminergic agent is administered to the subject in three doses per day during each treatment period.

348. The method of any one of claims 344-347, wherein the first, second, and/or third treatment period has a duration of from about 1 week to about 24 weeks.

349. The method of claim 348, wherein the first, second, and/or third treatment period has a duration of about 4 weeks.

350. The method of claim 1, wherein the second dopaminergic agent is administered to the subject:
(i) in an amount of from about 35 mg to about 100 mg per dose during a first treatment period;
(ii) in an amount of from about 50 mg to about 150 mg per dose during a second treatment period; and
(iii) in an amount of from about 150 mg to about 250 mg per dose during a third treatment period,
optionally wherein the second dopaminergic agent is administered to the subject in three doses per day during each treatment period.

351. The method of claim 350, wherein the second dopaminergic agent is administered to the subject:
(i) in an amount of from about 40 mg to about 75 mg per dose during a first treatment period;
(ii) in an amount of from about 75 mg to about 125 mg per dose during a second treatment period; and
(iii) in an amount of from about 175 mg to about 225 mg per dose during a third treatment period,
optionally wherein the second dopaminergic agent is administered to the subject in three doses per day during each treatment period.

352. The method of claim 351, wherein the second dopaminergic agent is administered to the subject:
(i) in an amount of from about 45 mg to about 55 mg per dose during a first treatment period;
(ii) in an amount of from about 85 mg to about 110 mg per dose during a second treatment period; and
(iii) in an amount of from about 190 mg to about 210 mg per dose during a third treatment period,
optionally wherein the second dopaminergic agent is administered to the subject in three doses per day during each treatment period.

353. The method of claim 352, wherein the second dopaminergic agent is administered to the subject:
(i) in an amount of about 50 mg per dose during a first treatment period;
(ii) in an amount of about 100 mg per dose during a second treatment period; and
(iii) in an amount of about 200 mg per dose during a third treatment period,
optionally wherein the second dopaminergic agent is administered to the subject in three doses per day during each treatment period.

354. The method of any one of claims 350-353, wherein the first, second, and/or third treatment period has a duration of from about 1 week to about 24 weeks.

355. The method of claim 354, wherein the first, second, and/or third treatment period has a duration of about 4 weeks.

356. The method of claim 1, wherein the analgesic agent, first dopaminergic agent, and second dopaminergic agent are administered to the subject in a single pharmaceutical composition.

357. The method of claim 1, wherein the analgesic agent is administered to the subject in a first pharmaceutical composition and the first dopaminergic agent and second dopaminergic agent are administered to the subject in a separate pharmaceutical composition.

358. The method of claim 1, wherein the analgesic agent is a non-steroidal anti-inflammatory drug (NSAID).

359. The method of claim 358, wherein the analgesic agent is naproxen, aceclofenac, acemetacin, acetaminophen, aloxiprin, aspirin, benorilate, bromfenac, celecoxib, deracoxib, diclofenac, diflunisal, ethenzamide, etodolac, etofenamate, etoricoxib, fenbufen, fenoprofen, flufenamic acid, flurbiprofen, lonazolac, lornoxicam, ibuprofen, indomethacin, isoxicam, kebuzone, ketoprofen, ketorolac, licofelone, loxoprofen, lumiracoxib, meclofenamic acid, mefenamic acid, meloxicam, metamizol, mofebutazone, naproxen, nabumetone, niflumic acid, nimesulide, oxaprozin, oxyphenbutazone, parecoxib, phenidone, phenylbutazone, piroxicam, propacetamol, propyphenazone, rofecoxib, salicylamide, sulfinpyrazone, sulindac, suprofen, tiaprofenic acid, tenoxicam, tolmetin, or valdecoxib.

360. The method of claim 359, wherein the analgesic agent is naproxen.

361. The method of claim 1, wherein the analgesic agent is an anticonvulsant.

362. The method of claim 361, wherein the analgesic agent is pregabalin, lamotrigine, topiramate, oxcarbazepine, tiagabine, levetiracetam, zonisamide, phenytoin, carbamazepine, gabapentin, or ethosuximide.

363. The method of claim 1, wherein the analgesic agent is paracetamol.

364. The method of claim 1, wherein the first dopaminergic agent and/or second dopaminergic agent is a dopamine receptor agonist or a precursor thereof.

365. The method of claim 1, wherein the first dopaminergic agent is a D2 agonist.

366. The method of claim 365, wherein the first dopaminergic agent is pramipexole or carbidopa.

367. The method of claim 366, wherein the first dopaminergic agent is carbidopa.

368. The method of claim 1, wherein the second dopaminergic agent is a D1 agonist.

369. The method of claim 368, wherein the second dopaminergic agent is levodopa.

370. The method of claim 1, the method comprising administering an additional therapeutic agent to the subject.

371. The method of claim 370, wherein the additional therapeutic agent is a chemotherapeutic agent, an anti-inflammatory agent, an antidepressant, an antiemetic agent, or a muscle relaxant.

372. The method of claim 1, wherein the analgesic agent, first dopaminergic agent, and/or second dopaminergic agent is administered to the patient orally, intravenously, intramuscularly, intravitreally, ocularly, intraocularly, itraorbitally, intradermally, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, topically, by inhalation, by injection, by implantation, by infusion, by continuous infusion, by localized perfusion bathing target cells directly, by catheter, by lavage, in creams, or in lipid compositions.

373. The method of claim 372, wherein the analgesic agent, first dopaminergic agent, and/or second dopaminergic agent is administered to the patient orally.

374. The method of claim 1, wherein the pain is acute pain.

375. The method of claim 374, wherein the acute pain is caused by a sports-related injury, a military injury, other physical trauma, a surgical procedure, cancer, infection, inflammation, or childbirth.

376. The method of claim 1, wherein the subject is at risk of transitioning from experiencing acute pain to experiencing chronic pain.

377. The method of claim 1, wherein the method prevents transition of acute pain to chronic pain.

378. The method of claim 1, wherein the pain is chronic pain.

379. The method of any one of claims 376-378, wherein the chronic pain is peripheral neuropathic pain, post-herpetic neuralgia, diabetic neuropathic pain, neuropathic cancer pain, failed back-surgery syndrome, trigeminal neuralgia, phantom limb pain, central neuropathic pain, multiple sclerosis related pain, Parkinson disease-related pain, post-stroke pain, post-traumatic spinal cord injury pain, pain from dementia, musculoskeletal pain, osteoarthritic pain, fibromyalgia syndrome, inflammatory pain, rheumatoid arthritis, endometriosis, migraine, cluster headache, tension headache syndrome, facial pain, headache caused by other diseases, visceral pain, interstitial cystitis, irritable bowel syndrome, chronic pelvic pain syndrome, lower back pain, neck and shoulder pain, burning mouth syndrome, or complex regional pain syndrome.

380. The method of claim 1, wherein administration of the analgesic agent, first dopaminergic agent, and second dopaminergic agent commences prior to the onset of the pain.

381. The method of claim 380, wherein administration of the analgesic agent, first dopaminergic agent, and second dopaminergic agent commences no earlier than two months prior to the onset of the pain.

382. The method of claim 1, wherein administration of the analgesic agent, first dopaminergic agent, and second dopaminergic agent commences after the onset of the pain.

383. The method of claim 382, wherein administration of the analgesic agent, first dopaminergic agent, and second dopaminergic agent commences no later than 3 months after the onset of the pain.

384. The method of claim 1, wherein the subject is a mammal.

385. The method of claim 384, wherein the subject is a human.

386. The method of claim 1, wherein the subject is a female.

387. The method of claim 1, wherein the subject is a male.

388. The method of claim 1, wherein the method further comprises performing a brain scan on the subject, optionally wherein the brain scan reveals that the subject has reduced connectivity between the subject's nucleus accumbens (NAc) and the subject's medial prefrontal cortex (mPFC) relative to a reference level of NAc-mPFC connectivity.

389. The method of claim 1, wherein the subject has been identified as having a reduced connectivity between the subject's nucleus accumbens (NAc) and the subject's medial prefrontal cortex (mPFC) relative to a reference level of NAc-mPFC connectivity.

390. The method of claim 388 or 389, wherein the brain scan is a functional magnetic resonance imaging (fMRI) technique.

391. The method of claim 388 or 389 wherein the reference level is the median level of NAc-mPFC connectivity in a population of patients being tested for likelihood of responding to administration of the analgesic agent, first dopaminergic agent, and/or second dopaminergic agent.

392. The method of claim 388 or 389, wherein the reference level is a z-transformed correlation score of NAc-mPFC connectivity, optionally wherein the subject's NAc-mPFC connectivity is compared to the reference level by assessing the subject's NAc-mPFC z-transformed correlation score.

393. The method of claim 392, wherein the reference level is from about 0.1 to about 0.4.

394. The method of claim 393, wherein the reference level is from about 0.15 to about 0.3.

395. The method of claim 394, wherein the reference level is about 0.2.

396. The pharmaceutical composition of claim 179, comprising the second dopaminergic agent in an amount of from about 35 mg to about 100 mg.

397. The pharmaceutical composition of claim 396, comprising the second dopaminergic agent in an amount of from about 40 mg to about 75 mg.

398. The pharmaceutical composition of claim 397, comprising the second dopaminergic agent in an amount of from about 45 mg to about 55 mg.

399. The pharmaceutical composition of claim 398, comprising the second dopaminergic agent in an amount of from about 50 mg.

400. The pharmaceutical composition of claim 179, comprising the second dopaminergic agent in an amount of from about 50 mg to about 150 mg.

401. The pharmaceutical composition of claim 400, comprising the second dopaminergic agent in an amount of from about 75 mg to about 125 mg.

402. The pharmaceutical composition of claim 401, comprising the second dopaminergic agent in an amount of from about 85 mg to about 110 mg.

403. The pharmaceutical composition of claim 402, comprising the second dopaminergic agent in an amount of from about 100 mg.

404. The pharmaceutical composition of claim 179, comprising the second dopaminergic agent in an amount of from 150 mg to about 250 mg.

405. The pharmaceutical composition of claim 404, comprising the second dopaminergic agent in an amount of from about 175 mg to about 225 mg.

406. The pharmaceutical composition of claim 405, comprising the second dopaminergic agent in an amount of from about 190 mg to about 210 mg.

407. The pharmaceutical composition of claim 406, comprising the second dopaminergic agent in an amount of from about 200 mg.

408. The pharmaceutical composition of claim 179, wherein the analgesic agent is a NSAID.

409. The pharmaceutical composition of claim 408, wherein the analgesic agent is naproxen, aceclofenac, acemetacin, acetaminophen, aloxiprin, aspirin, benorilate, bromfenac, celecoxib, deracoxib, diclofenac, diflunisal, ethenzamide, etodolac, etofenamate, etoricoxib, fenbufen, fenoprofen, flufenamic acid, flurbiprofen, lonazolac, lornoxicam, ibuprofen, indomethacin, isoxicam, kebuzone, ketoprofen, ketorolac, licofelone, loxoprofen, lumiracoxib, meclofenamic acid, mefenamic acid, meloxicam, metamizol, mofebutazone, naproxen, nabumetone, niflumic acid, nimesulide, oxaprozin, oxyphenbutazone, parecoxib, phenidone, phenylbutazone, piroxicam, propacetamol, propyphenazone, rofecoxib, salicylamide, sulfinpyrazone, sulindac, suprofen, tiaprofenic acid, tenoxicam, tolmetin, or valdecoxib.

410. The pharmaceutical composition of claim 409, wherein the analgesic agent is naproxen.

411. The pharmaceutical composition of claim 179, wherein the analgesic agent is an anticonvulsant.

412. The pharmaceutical composition of claim 411, wherein the analgesic agent is pregabalin, lamotrigine, topiramate, oxcarbazepine, tiagabine, levetiracetam, zonisamide, phenytoin, carbamazepine, gabapentin, or ethosuximide.

413. The pharmaceutical composition of claim 179, wherein the analgesic agent is paracetamol.

414. The pharmaceutical composition of claim 179, wherein the first dopaminergic agent and/or second dopaminergic agent is a dopamine receptor agonist or a precursor thereof.

415. The pharmaceutical composition of claim 179, wherein the first dopaminergic agent is a D2 agonist.

416. The pharmaceutical composition of claim 415, wherein the first dopaminergic agent is pramipexole or carbidopa.

417. The pharmaceutical composition of claim 416, wherein the first dopaminergic agent is carbidopa.

418. The pharmaceutical composition of claim 179, wherein the second dopaminergic agent is a D1 agonist.

419. The pharmaceutical composition of claim 418 wherein the second dopaminergic agent is levodopa.

420. The pharmaceutical composition of claim 179, wherein the composition is a tablet, capsule, gel cap, powder, liquid solution, or liquid suspension.

421. The pharmaceutical composition of claim 179, wherein the composition is formulated for sustained release.

422. The pharmaceutical composition of claim 179, wherein the composition is an implanted device.

423. A kit comprising the pharmaceutical composition of claim 179 and a package insert instructing a user of the kit to administer the pharmaceutical composition to a subject at risk of acute or chronic pain, having acute or chronic pain, or at risk of transitioning from acute pain to chronic pain.

424. A kit comprising an analgesic agent, a first dopaminergic agent, a second dopaminergic agent, and a package insert instructing a user of the kit to administer the analgesic agent, first dopaminergic agent, and second dopaminergic agent to a subject in accordance with the method of claim 1.
